(19) United States
(12) Patent Application Publication
(10) Pub. No.: US 2012/0040415 A1
(43) Pub. Date: Feb. 16, 2012

(54) METHOD FOR PRODUCING AN L-AMINO ACID

(76) Inventors: Yuichi Nakahara, Kawasaki-shi (JP); Yusuke Hagiwara, Kawasaki-shi (JP); Yuri Nagai, Kawasaki-shi (JP); Takuji Ueda, Kawasaki-shi (JP); Kazuyuki Hayashi, Kawasaki-shi (JP); Yuki Yano, Kawasaki-shi (JP)

(21) Appl. No.: 13/170,661

(22) Filed: Jun. 28, 2011

Related U.S. Application Data
(63) Continuation of application No. PCT/JP10/51071, filed on Jan. 21, 2010.
(60) Provisional application No. 61/172,948, filed on Apr. 27, 2009.

(30) Foreign Application Priority Data
Jan. 23, 2009 (JP) 2009-013123
Nov. 13, 2009 (JP) 2009-259654

(51) Int. Cl. C12P 13/08; C12P 13/04

(52) U.S. Cl. 435/115; 435/106

(57) ABSTRACT

A method is provided for producing an L-amino acid which includes the steps of culturing a bacterium, which belongs to the family Enterobacteriaceae and is able to produce an L-amino acid, in a medium containing glycerol as a carbon source to produce and accumulate an L-amino acid in the medium, and collecting the L-amino acid from the culture. The culture is performed as a fed-batch culture or a continuous culture, and a feed medium containing glycerol is added to the fermentation medium so that the glycerol concentration in the fermentation medium is 5 g/L or higher.

This application is a Continuation of, and claims priority under 35 U.S.C. §120 to, International Application No. PCT/JP2010/051071, filed Jan. 21, 2010, and claims priority therethrough under 35 U.S.C. §119 to Japanese Patent Application No. 2009-013123, filed Jan. 23, 2009, U.S. Provisional Patent Application No. 61/172,948, filed Apr. 27, 2009, and Japanese Patent Application No. 2009-259654, filed Nov. 13, 2009, the entireties of which are incorporated by reference herein. Also, the Sequence Listing filed electronically herewith is hereby incorporated by reference (File name: 2011-06-28T_US-423_Seq_List; File size: 425 KB; Date recorded: Jun. 28, 2011).

### BACKGROUND OF THE INVENTION

1. Technical field

The present invention relates to a method for producing an L-amino acid using a bacterium. More precisely, this method for producing an L-amino acid uses glycerol as a carbon source. L-Amino acids are industrially useful as additives for animal feed, components of health food, amino acid infusions, and so forth.

2. Background Art

L-Amino acids are industrially produced by fermentation using microorganisms belonging to the genus Brevibacterium, Corynebacterium, Escherichia, and the like. In such production methods, strains can be used which are isolated from nature, or artificial variants of such strains can be used. Furthermore, microorganism strains can be used which are modified by a recombinant DNA technique so that the activity of a basic L-amino acid biosynthesis enzyme is increased, and so forth (EP 0643135 B, EP 0733712 B, EP 1477565 A, EP 0796912 A, EP 0837134 A, WO01/53459, EP 1170376 A, WO2005/010175, WO96/17930).

When L-amino acids are produced using microorganisms, sugars are generally used as a main component of the substrate, but glycerol can also be used as a substrate, similar to sugars (EP 1715055 A, EP 1715056 A).

It is known that Escherichia coli has a plurality of pathways which participate in the glycerol metabolism. One of these pathways starts with glycerol entering into the glycolysis system via glycerol-3-phosphate, and glycerol kinase and glycerol-3-phosphate dehydrogenase are responsible for the conversion. Furthermore, another pathway is known which starts with glycerol entering into the glycolysis system via dihydroxyacetone, and its first reaction is catalyzed by glycerol dehydrogenase.

It has been revealed that the major glycerol assimilation pathway of E. coli is the former pathway, since a mutant strain deficient in glpK (the gene coding for glycerol kinase) or glpD (the gene coding for glycerol-3-phosphate dehydrogenase), cannot grow in a medium containing glycerol as the sole carbon source (J. Bacteriol., 23, 8259-8271 (2006)).

Furthermore, it is known that if the glycerol dehydrogenase activity is increased when screening using a mutant strain deficient in glpK, glpD and glpR, which is a repressor gene of the glp regulon, the strain recovers from lethality in a medium containing glycerol as a sole carbon source (J. Bacteriol., 131, 1026-1028 (1977)).

To date, the pathway via glycerol-3-phosphate has been thought to be the main glycerol assimilation pathway of microorganisms belonging to the family Enterobacteriaceae as described above, and the glycerol assimilation pathway via dihydroxyacetone has been thought to be an unnecessary pathway for glycerol assimilation of microorganisms belonging to the family Enterobacteriaceae.

It has been considered that, in a conventional L-amino acid production culture using a sugar as the carbon source, it is desirable to keep the concentration of the sugar low (WO05/14841, WO05/14842, WO05/014843). Therefore, apparatuses for controlling sugar concentration during culture at a low concentration of sugar and so forth have been developed (U.S. Pat. No. 5,912,113 B). Furthermore, although it is known that the Km value of glycerol dehydrogenase for glycerol is high (J. Biol. Chem., 259 (4), 2124-2129), the influence of glycerol concentration in the culture medium on substance production using glycerol as a carbon source has not been examined.

### SUMMARY OF THE INVENTION

An aspect of the present invention is to provide a method for producing an L-amino acid by fermentation using a substrate containing glycerol, which is improved as compared to conventional techniques.

When a culture is performed while controlling glycerol concentration in the medium to be 5 g/L or higher, the productivity of L-amino acids can be improved.

It is an aspect of the present invention to provide a method for producing an L-amino acid comprising culturing a bacterium which belongs to the family Enterobacteriaceae and is able to produce an L-amino acid, in a fermentation medium containing glycerol, and collecting the L-amino acid from the fermentation medium, wherein the culturing is performed as a fed-batch culture or a continuous culture, and a feed medium comprising glycerol is added to the fermentation medium so that the glycerol concentration of the fermentation medium is 5 g/L or higher.

It is a further aspect of the present invention to provide the method as described above, wherein the glycerol concentration is 5 g/L or higher for a period which is at least 50% or more of the entire culture period.

It is a further aspect of the present invention to provide the method as described above, wherein the culturing comprises a proliferation phase during which the bacterium proliferates, and a production phase during which L-amino acid is produced, wherein the production phase occurs after the proliferation phase, and wherein the production phase is at least 70% or more of the entire culture period, and the glycerol concentration is 5 g/L or higher for a period which is at least 70% or more of the production phase.

It is a further aspect of the present invention to provide the method as described above, wherein an activity of an enzyme of the dihydroxyacetone pathway has been enhanced in the bacterium.

It is a further aspect of the present invention to provide the method as described above, wherein the enzyme is selected from the group consisting of glycerol dehydrogenase, dihydroxyacetone kinase, fructose-6-phosphate aldolase, and combinations thereof.

It is a further aspect of the present invention to provide the method as described above, wherein the enzyme comprises a mixture of glycerol dehydrogenase, dihydroxyacetone kinase, and fructose-6-phosphate aldolase.

It is a further aspect of the present invention to provide the method as described above, wherein the activity of the enzyme has been enhanced by increasing the copy number of a gene coding for the enzyme, or by modifying an expression control sequence of the gene.

It is a further aspect of the present invention to provide the method as described above, wherein the bacterium is an Escherichia bacterium.

It is a further aspect of the present invention to provide the method as described above, wherein the bacterium is Escherichia coli.

It is a further aspect of the present invention to provide the method as described above, wherein the L-amino acid is L-lysine or L-threonine.

### BRIEF DESCRIPTION OF THE DRAWINGS

FIG. 1 shows the change in glycerol concentration over time.

indicates a glycerol concentration of 0 to 5 g/L, ▴ indicates a glycerol concentration of 10 to 15 g/L, and

▪ indicates a glycerol concentration of 15 to 20 g/L (the same shall apply to FIGS. 2 and 3).

FIG. 2 shows the change in L-lysine accumulation over time.

FIG. 3 shows the change in cell growth over time.

FIG. 4 shows the change in glycerol concentration over time.

indicates a glycerol concentration of 0 to 5 g/L,

▴ indicates a glycerol concentration of 5 to 10 g/L, and

▪ indicates a glycerol concentration of 10 to 15 g/L (the same shall apply to FIG. 5).

FIG. 5 shows the change in cell growth over time.

### DETAILED DESCRIPTION OF THE PREFERRED EMBODIMENTS

Hereafter, the present invention will be explained in detail.

<1> Method of the Present Invention

The method in accordance with the presently disclosed subject matter is a method for producing an L-amino acid which includes the steps of culturing a microorganism which belongs to the family Enterobacteriaceae and is able to produce an L-amino acidin a fermentation medium containing glycerol as a carbon source, resulting in accumulation of an L-amino acid in the medium, and collecting the L-amino acid from the medium, wherein the culturing is performed as a fed-batch culture or a continuous culture, and a feed medium containing glycerol is added to the fermentation medium so that the glycerol concentration in the fermentation medium is at least 5 g/L or higher.

The ability to produce an L-amino acid (L-amino acid-producing ability) can mean the ability of a microorganism in accordance with the presently disclosed subject matter to produce and cause accumulation of an L-amino acid in the medium or cells when cultured in the medium. An exemplary microorganism can be able to produce two or more kinds of L-amino acids. Although the ability to produce an L-amino acid may be inherent in the microorganism, such an ability can be obtained by modifying such microorganisms as mentioned below using a mutagenesis method or a recombinant DNA technique so that they become able to produce L-amino acid.

Although the type of the L-amino acid is not particularly limited, examples include basic amino acids such as L-lysine, L-ornithine, L-arginine, L-histidine and L-citrulline; aliphatic amino acids such as L-isoleucine, L-alanine, L-valine, L-leucine and L-glycine; amino acids which are hydroxy-monoaminocarboxylic acids such as L-threonine and L-serine; cyclic amino acids such as L-proline; aromatic amino acids such as L-phenylalanine, L-tyrosine and L-tryptophan; sulfur-containing amino acids such as L-cysteine, L-cystine, and L-methionine; acidic amino acids such as L-glutamic acid and L-aspartic acid; and amino acids with an amide group at the side chain such as L-glutamine and L-asparagine. An exemplary microorganism can be able to produce two or more kinds of L-amino acids.

In the present invention, the term “L-amino acid” includes L-amino acids in a free form and salts thereof, such as sulfates, hydrochlorides, and carbonates.

The medium or the culture can contain glycerol as the carbon source, and in addition, can contain counter ions that are required for L-amino acid fermentation, a nitrogen source, sulfur, phosphorus, etc. required for growth of the chosen microorganism. A substance serving as a supply source of the counter ions can be a substance containing ammonium ions that serve as a nitrogen source; sulfate ions, chloride ions, or carbonate ions that serve as counter ions of carboxylic acid, organic acids, amino acids, and so forth.

The concentration of glycerol in the medium can be controlled by adding a feed medium containing glycerol. That is, the culture can be performed as a fed-batch culture or a continuous culture. A fed-batch culture is a culture method in which the medium is continuously or intermittently added into the culture vessel during the culture, and the medium is not extracted until the end of the culture. A continuous culture is a method in which the medium is continuously or intermittently added into the culture vessel during culture, and the medium is extracted from the vessel (usually in a volume equal to the volume of the amount added) at the same time. However, glycerol may be present in the starting medium.

The “starting medium” can mean a medium used in a batch culture before adding the feed medium to the fed-batch culture or continuous culture, that is, the medium used at the start of the culture. The “feed medium” can mean a medium which is supplied to the fermentation tank in the fed-batch culture or continuous culture. The feed medium is not particularly limited so long as it contains glycerol, and it may include all or just some of the components required for growth of the microorganism. It may also contain only glycerol.

The term “fermentation medium” means the medium present in a fermentation tank, and the L-amino acid is collected from this fermentation medium. Furthermore, the term “fermentation tank” means a vessel in which the L-amino acid fermentation is performed. For example, a typical fermentation tank or a jar fermenter may be used. Furthermore, the volume of the fermentation tank is not limited so long as an L-amino acid can be produced and collected.

The glycerol concentration in the medium in the fermentation tank can be controlled during the culture to be 5 g/L or higher. The glycerol concentration in the medium can be controlled by the adding glycerol so that the concentration is 5 g/L or higher, or 7 g/L or higher, or even 10 g/L or higher. Although the upper limit of the glycerol concentration in the medium is not defined so long as the L-amino acid is efficiently produced, the glycerol concentration can be, for example, 25 g/L or lower, or 20 g/L or lower, or even 15 g/L or lower.

The glycerol concentration can be controlled so that it is constant for the entire culture period. However, it can also be controlled for only a portion of the culture period.

The glycerol concentration can be controlled for a period corresponding to 50% or more, or 70% or more, or even 80% or more, of the entire culture period.

When the method includes a period of proliferation of the chosen bacterium (proliferation phase), and a period of production of the L-amino acid (production phase), the glycerol concentration can be controlled so that it is not lower than a certain level during the production phase. The “proliferation phase” can mean a period when the carbon source is primarily used for cell growth, and is usually within about 5 hours, or 10 hours, or even 15 hours, from the start of the culture, that is, the period when the microorganism is logarithmically proliferating. The “production phase” can mean the period of the culture following the proliferation phase, when the carbon source is mainly used for production of the L-amino acid, and is usually , for example, from about 5 hours, or 10 hours, or even 15 hours, after the start of the culture until the end of culture. The production phase can encompass 60% or more, or 70% or more, or even 80% or more, of the entire culture period (from the start to the end of the culture).

The glycerol concentration does not need to be within the aforementioned range over the entire period of the production phase, and when the concentration is below the aforementioned range for a certain period of time, it may be increased by the timely addition of glycerol. Moreover, glycerol may be intermittently supplemented when the concentration is insufficient. The glycerol concentration of the medium in the fermentation tank can be controlled for 50% or more, or 70% or more, or even 80% or more, of the production phase.

In the proliferation phase, the glycerol concentration may be controlled to be within the aforementioned range, or may be at a concentration lower than the aforementioned range.

So long as the glycerol concentration of the medium in a fermentation tank is maintained to be within the aforementioned range, the feed medium may contain glycerol in a necessary amount, or glycerol may temporarily run short in the feed medium. The term “temporarily” can mean, for example, 15% or less, or 10% or less, or even 5% or less, of the production phase. When glycerol runs short, the glycerol concentration may temporarily be zero. During L-amino acid production, in particular, the concentration of the carbon source can be zero at the end of culture in view of the subsequent processing step. The end of culture can mean a time point when the L-amino acid concentration reaches an intended concentration, or the culture for a predetermined period is completed. After the end of culture, usually the next step is the collection of the amino acid from fermentation medium.

The glycerol concentration can be controlled by, for example, measuring the glycerol concentration in the medium, and when it becomes lower than a certain concentration, adding a feed medium containing glycerol to the fermentation tank. The glycerol concentration can be measured with, for example, BF-5 produced by Oji Scientific Instruments. If the conditions to maintain the glycerol concentration within a predetermined range, including time and volume of the additional feed medium, are determined or known for the chosen strain, the culture can be performed using those same conditions, and measuring the glycerol concentration and controlling it on the basis of the measurement becomes unnecessary.

The glycerol in the medium can be the sole carbon source, or a mixed medium can be used which contains other carbon sources in addition to glycerol. Examples of the other carbon sources can include saccharides such as glucose, fructose, sucrose, lactose, galactose, blackstrap molasses, or sugar solutions obtained by hydrolysis of starch hydrolysate or biomass, alcohols such as ethanol, and organic acids such as fumaric acid, citric acid, and succinic acid. When two or more types of carbon sources are used, the amount of glycerol relative to the amount of all the carbon sources can be 50% or more, 60% or more, 70% or more, 80% or more, or even 90% or more.

The glycerol may be reagent grade, or may be crude glycerol. Examples of the crude glycerol can include glycerol produced as a by-product of biodiesel fuel (Mu, Y., et al, Biotechnol Lett., 28, 1755-91759 (2006); Haas M. J., et al., Bioresour. Technol., 97, 4, 671-8678 (2006); WO2008/102861).

When crude glycerol is used, the glycerol concentration can mean the concentration of the glycerol itself.

As for other components which can be added to the medium, besides the carbon source, conventional medium components such as a nitrogen source, inorganic ions, and other organic components as required can be used.

As the nitrogen source, ammonia, ammonium salts such as ammonium sulfate, ammonium carbonate, ammonium chloride, ammonium phosphate, ammonium acetate and urea, nitrates, and so forth can be used. Ammonia gas and aqueous ammonia that are used to adjust the pH can also be utilized as the nitrogen source. Furthermore, peptone, yeast extract, meat extract, malt extract, corn steep liquor, soybean hydrolysate, and so forth can also be utilized. The medium can contain one or more of these nitrogen sources. These nitrogen sources can also be used for both the starting medium and the feed medium. Furthermore, the same nitrogen source can be used for both the starting medium and the feed medium, or the nitrogen source of the feed medium may be different from that of the starting medium.

The medium can contain a phosphoric acid source and a sulfur source in addition to the carbon source and the nitrogen source. As the phosphoric acid source, potassium dihydrogenphosphate, dipotassium hydrogenphosphate, phosphate polymers such as pyrophosphoric acid, and so forth can be utilized. The sulfur source may be any sulfur source so long as it has a sulfur atom, and can include salts of sulfuric acid such as sulfates, thiosulfates and sulfites and sulfur-containing amino acids such as cysteine, cystine and glutathione. Among these, ammonium sulfate is one example.

Furthermore, the medium can contain a growth-promoting factor, such as a nutrient having a growth-promoting effect, in addition to the carbon source, the nitrogen source, and sulfur. As the growth-promoting factor, trace metals, amino acids, vitamins, nucleic acids as well as peptone, casamino acid, yeast extract, soybean protein degradation product, and so forth containing the foregoing substances can be used. Examples of the trace metals include iron, manganese, magnesium, calcium, and so forth. Examples of the vitamins include vitamin B₁, vitamin B₂, vitamin B₆, nicotinic acid, nicotinic acid amide, vitamin B₁₂, and so forth.

Furthermore, when an auxotrophic mutant that requires an amino acid or the like for growth is used, the required nutrients can be added to the medium. In particular, since the L-lysine biosynthetic pathway is enhanced and the L-lysine degrading ability is attenuated in many L-lysine-producing bacteria as described below, one or more types of substances including L-threonine, L-homoserine, L-isoleucine and L-methionine can be added.

The aforementioned components may be present in the starting medium or the feed medium, or both.

The starting medium and the feed medium can have the same or different compositions. Furthermore, the starting medium and the feed medium may have the same or different concentrations of each component. Furthermore, when the feed medium is added at multiple stages, the compositions of the feed media added at the various times may be the same or different.

The culture can be performed as an aeration culture at a fermentation temperature of 20 to 45° C., or at 33 to 42° C. The oxygen concentration is adjusted to 5 to 50%, or about 10%. Furthermore, the aeration culture can be performed with the pH adjusted to 5 to 9. If the pH drops during the culture, for example, calcium carbonate or an alkali such as an ammonia gas and aqueous ammonia can be added to neutralize the culture. When the culture is performed under such conditions for about 10 to 120 hours, a marked amount of L-amino acid is produced in the culture medium. Although the concentration of L-amino acid which accumulates is not limited so long as the L-amino acid can be isolated and collected from the medium, it can be 50 g/L or higher, 75 g/L or higher, or even 100 g/L or higher.

The L-amino acid can be collected by a known collection method from the culture medium after the culture. For example, the cells can be removed from the culture medium by centrifugation or the like, and then the L-amino acid can be crystallized by concentration, and the L-amino acid can be collected.

The culture of the microorganism can be performed as a seed culture and a main culture in order to ensure accumulation of a certain amount of L-amino acid. The seed culture can be performed as a shaking culture using a flask or the like, or a batch culture, and the main culture can be performed as a fed-batch culture or a continuous culture.

When the continuous culture method is used, the medium may be extracted and added simultaneously, or a part of the medium may be extracted, and then the medium may be added. Furthermore, the method may also be a continuous culture method which can include extracting the culture medium containing the L-amino acid and bacterial cells, and returning only the cells to the fermentation tank to reuse the cells (French Patent No. 2669935). As the method of continuously or intermittently feeding a nutrient source, the same method as used in the fed-batch culture is used.

The continuous culture method of reusing bacterial cells includes intermittently or continuously extracting the fermentation medium when the amino acid concentration reaches a predetermined level, extracting only the L-amino acid and re-circulating filtration residues containing bacterial cells into the fermentation tank. (see French Patent No. 2669935).

When the culture medium is intermittently extracted, a portion of the amount of L-amino acid can be extracted when the L-amino acid concentration reaches a predetermined level, and fresh medium is added to continue the culture. Furthermore, as for the volume of the medium to be added, the culture can be performed so that the final volume after the addition of the medium is equal to the volume of the culture medium before the extraction. The term “equal” can mean that the volume corresponds to about 93 to 107% of the volume of the culture medium before the extraction.

When the culture medium is continuously extracted, the extraction can be started at the same time as, or after the addition of, the nutrient medium. For example, the extraction can be started within 5 hours, 3 hours, or even 1 hour, after the start of the addition of the nutrient medium. Furthermore, the volume of the culture medium which is extracted can be equal to the volume of the medium which is then added.

When a basic amino acid such as L-lysine is produced, the fermentation can be performed by controlling the pH of the medium during culture to be 6.5 to 9.0, and the pH of the medium at the end of the culture to be 7.2 to 9.0, and the culture period can be when the medium contains 20 mM or more of bicarbonate ions and/or carbonate ions, so that these bicarbonate ions and/or carbonate ions serve as counter ions for the basic amino acid, and the objective basic amino acid is then collected (Japanese Patent Laid-open No. 2002-65287, U.S. Patent Published Application No. 2002/0025564, EP 1813677 A).

When a microorganism which is able to produce a basic amino acidis cultured in a medium under aerobic conditions, carbonate ions, bicarbonate ions, or both can be used as the major counter ions of the basic amino acid. To provide carbonate ions and/or bicarbonate ions in an amount required to serve as counter ions of the basic amino acid, the pH of the medium should be controlled to be 6.5 to 9.0, or 6.5 to 8.0, during the culture, and should be 7.2 to 9.0 at the end of the culture. The pressure in the fermentation tank can be controlled so that it is positive during fermentation, or carbon dioxide or a mixed gas containing carbon dioxide can be supplied into the medium (Japanese Patent Laid-open No. 2002-65287, U.S. Patent Published Application No. 2002/0025564, EP 1813677 A).

The pressure in the fermentation tank may be controlled to be positive during the fermentation, and at the same time, carbon dioxide gas or a mixed gas containing carbon dioxide gas may be supplied to the medium. Both the above operations can be performed so that there is a culture period when 20 mM or more, 30 mM or more, or even 40 mM or more, of bicarbonate ions and/or carbonate ions are present in the medium. The internal pressure of the fermentation tank, supply amount of carbon dioxide or mixed gas containing carbon dioxide, or the limited gas supply volume can be determined by, for example, measuring bicarbonate ions or carbonate ions in the medium, or the pH or ammonia concentration of the medium.

In the above embodiment, the pH of the medium is controlled to be 6.0 to 9.0, or 6.5 to 8.0, during the culture, and 7.2 to 9.0 at the end of the culture. It is possible to suppress the pH of the medium so that the amount of bicarbonate ions and/or carbonate ions present in the medium is lower as compared to the conventional methods. When the pH is controlled with ammonia, ammonia is supplied in order to increase the pH, and it can serve as a nitrogen source for the basic amino acid. Examples of cations other than the basic amino acid in the medium include K, Na, Mg, Ca etc. originating in medium components. These can be present in an amount of 50% or less of the total cations.

Furthermore, the internal pressure of the fermentation tank during fermentation can be made positive by, for example, making the gas supply pressure higher than the exhaust pressure. By making the internal pressure of the fermentation tank positive, the carbon dioxide generated by fermentation dissolves in the culture medium to generate bicarbonate ions or carbonate ions, and these can serve as counter ions of the basic amino acid. The internal pressure of the fermentation tank is, specifically, 0.03 to 0.2 MPa, 0.05 to 0.15 MPa, or 0.1 to 0.3 MPa, in terms of the gage pressure (pressure difference with respect to the atmospheric pressure). Moreover, by supplying carbon dioxide or a mixed gas containing carbon dioxide to the culture medium, carbon dioxide may be dissolved in the medium. Furthermore, when supplying carbon dioxide or a mixed gas containing carbon dioxide to the medium, the internal pressure of the fermentation tank may be adjusted to be positive.

The internal pressure of the fermentation tank may be adjusted to be positive by, for example, making the gas supply pressure higher than the exhaust pressure. Furthermore, when carbon dioxide is supplied to the medium, for example, pure carbon dioxide or a mixed gas containing 5% by volume or more of carbon dioxide may be bubbled into the medium.

The aforementioned methods for dissolving bicarbonate ions and/or carbonate ions in the medium may be used independently, or two or more of them may be used in combination.

In the conventional methods, a sufficient amount of ammonium sulfate or ammonium chloride is usually added to the medium to serve as counter anions of the basic amino acid to be produced. Sulfuric acid or hydrochloric acid decomposition products of proteins etc. can be added to the medium as a nutrient component, and thus the generated sulfate ions and chloride ions are present in the medium. Therefore, the concentration of the weakly acidic carbonate ions is extremely low during the culture, such as a ppm order. The above embodiment is characterized in that these sulfate ions and chloride ions are reduced, and the carbon dioxide released by the microorganism during fermentation is dissolved in the medium in the aforementioned fermentation environment and can be used as counter ions. Therefore, in the above embodiment, it is not required to add sulfate ions or chloride ions to the medium in an amount more than the amount required for the growth. An appropriate amount of ammonium sulfate or the like can be added to the medium at an early stage of the culture, and then terminated in the middle of the culture. Alternatively, ammonium sulfate or the like may be added while maintaining the balance with the dissolved carbonate ions or bicarbonate ions in the medium. Moreover, as a nitrogen source of the basic amino acid, ammonia may be added to the medium. Ammonia may be supplied to the medium independently, or together with other gases.

Lower concentrations of anions other than bicarbonate ions and/or carbonate ions can be in the medium so long as they are present in amounts that are required for the growth of the microorganism. Examples of such anions include chloride ions, sulfate ions, phosphate ions, ionized organic acids, hydroxide ions, and so forth. The total molar concentration of these other ions is usually 900 mM or lower, 700 mM or lower, 500 mM or lower, 300 mM or lower, or even 200 mM or lower.

To reduce the amounts of sulfate ions and/or chloride ions required is one of the objects of the above embodiment, and the total amount of sulfate ions or chloride ions, or both contained in the medium is usually 700 mM or lower, 500 mM or lower, 300 mM or lower, 200 mM or lower, or even 100 mM or lower.

If ammonium sulfate is added to a medium as a counter ion source of a basic amino acid, carbon dioxide in the culture medium is usually eliminated by sulfate ions. However, in the above embodiment, it is not necessary to add an excess amount of ammonium sulfate to the medium, and therefore carbon dioxide can be easily dissolved in the fermentation medium.

Furthermore, in the above embodiment, it is preferable to control the total ammonia concentration in the medium to such an extent that “production of the basic amino acid is not inhibited”. Examples of the conditions to attain such a purpose include, for example, when the yield and/or productivity is 50% or more, 70% or more, or even 90% or more, of the yield and/or productivity which is obtained under optimal conditions. Specifically, the total ammonia concentration in the medium is preferably 300 mM or lower, 250 mM or lower, or 200 mM or lower. The dissociation degree of the ammonia decreases as the pH becomes higher. Non-dissociating ammonia is more toxic to bacteria as compared to ammonium ions. Therefore, the upper limit of the total ammonia concentration should be determined depending on the pH of the culture medium. That is, as the pH of the culture medium increases, the acceptable total ammonia concentration decreases. Therefore, the aforementioned total ammonia concentration “which does not inhibit the basic amino acid production” can be determined for each specific pH value. However, the total ammonia concentration range that is acceptable at the highest pH level during the culture may be used as the upper limit of the total ammonia concentration throughout the entire culture period.

On the other hand, the total ammonia concentration which functions as a source of nitrogen required for growth of the microorganism and production of the basic substance is not particularly limited, and can be appropriately determined, so long as a reduced level of the nitrogen source which can result in continuous depletion of ammonia during the culture does not reduce productivity of the objective substance by the microorganism. For example, the ammonia concentration can be measured over time during the culture, and if ammonia in the medium is depleted, a small amount of ammonia may be added to the medium. Although the total ammonia concentration after the addition of ammonia is not particularly limited, the total ammonia concentration may be, for example, 1 mM or higher, 10 mM or higher, or even 20 mM or higher.

The L-amino acid can be collected from the fermentation medium by a combination of known methods including the ion-exchange resin method (Nagai, H. et al., Separation Science and Technology, 39(16), 3691-3710), precipitation method, membrane separation method (Japanese Patent Laid-open Nos. 9-164323 and 9-173792), crystallization method (WO2008/078448, WO2008/078646), and so forth. When the L-amino acid accumulates in the cells, for example, the cells can be disrupted by ultrasonication or the like, and the supernatant can be obtained by removing the cells by centrifugation. The L-amino acid can then be collected from the supernatant by the ion-exchange resin method or the like.

The collected L-amino acid may contain bacterial cells, medium components, moisture, and by-product metabolites of the bacterium in addition to the L-amino acid. Purity of the collected L-amino acid can be 50% or higher, 85% or higher, or even 95% or higher (Japanese Patent No. 1214636, U.S. Pat. Nos. 5,431,933, 4,956,471, 4,777,051, 4,946,654, 5,840,358, 6,238,714, U.S. Patent Published Application No. 2005/0025878).

When the L-amino acid is precipitated in the medium, it can be collected by centrifugation or filtration. When the L-amino acid is precipitated in the medium, the L-amino acid dissolved in the medium can be crystallized, and then they can be collected together.

<2> Bacteria Belonging to the Family Enterobacteriaceae

The microorganism can belong to the family Enterobacteriaceae. It can have the ability to produce an L-amino acid, and be capable of producing the L-amino acid by using glycerol as the carbon source.

Typical examples of microorganisms belonging to the family Enterobacteriaceae include Escherichia bacteria and Pantoea bacteria. Other examples of microorganisms belonging to the family Enterobacteriaceae include γ-proteobacteria such as bacteria of the genus Enterobacter, Klebsiella, Serratia, Erwinia, Salmonella, Morganella, or the like.

A strain in which the activity of an enzyme of the dihydroxyacetone pathway (DHA pathway) is enhanced can be used. In the dihydroxyacetone pathway, glycerol is incorporated into cells, then converted into dihydroxyacetone, and enters the glycolysis system. In particular, glycerol dehydrogenase, dihydroxyacetone kinase, and fructose-6-phosphate aldolase are all enzymes of the DHA pathway. The DHA pathway separates into two pathways at the point when the dihydroxyacetone is generated from glycerol by the glycerol dehydrogenase reaction. One pathway enters into the glycolysis system via dihydroxyacetone phosphate, which is generated from dihydroxyacetone by dihydroxyacetone kinase, and the other enters into the glycolysis system via fructose-6-phosphate, which is generated from dihydroxyacetone by fructose-6-phosphate aldolase. Although the activity of any of glycerol dehydrogenase, dihydroxyacetone kinase, and fructose-6-phosphate aldolase can be enhanced, the combination of glycerol dehydrogenase and dihydroxyacetone kinase, the combination of glycerol dehydrogenase, and fructose-6-phosphate aldolase, and the combination of three of these enzymes are examples. All three enzymes' activities can also be enhanced.

“Glycerol dehydrogenase” can mean an enzyme which reversibly catalyzes the following oxidation reaction that converts glycerol into dihydroxyacetone by using NAD as a coenzyme (EC:1.1.1.6).

Glycerol+NAD→Dihydroxyacetone+NADH+H⁺

The phrase “modified to increase the glycerol dehydrogenase activity” can mean that the number of the glycerol dehydrogenase molecules per cell can be increased as compared to that of a wild-type or non-modified strain, or that the activity of the glycerol dehydrogenase per molecule can be improved as compared to that of a wild-type or non-modified strain. Moreover, when the enzyme activity is undetectable in a wild-type strain, and it is improved to a detectable level, the activity is also considered to be “increased”. The glycerol dehydrogenase activity can be at any level so long as it is detectable, but glycerol dehydrogenase can be modified so that the activity is 0.05 U/mg or higher, 0.25 U/mg or higher, or 0.5 U/mg or higher. Examples of wild-type strains of microorganisms belonging to the family Enterobacteriaceae which can serve as a reference for comparison include the Escherichia coli MG1655 strain (ATCC No. 47076) and W3110 strain (ATCC No. 27325), Pantoea ananatis AJ13335 strain (FERM BP-6615), and so forth. The glycerol dehydrogenase activity can be measured by referring to the method of Ansis, R. E. et al. (J. Biol. Chem., 2-3, 153-159 (1953))

“Dihydroxyacetone kinase” is an enzyme which reversibly catalyzes the following reaction that converts dihydroxyacetone into dihydroxyacetone phosphate, and either ATP (EC 2.7.1.29) or PEP (phosphoenolpyruvate) (EC 2.7.1.29) is used as a phosphate donor (Cell. Mol. Life Sci., 63 (2006) 890-900; Biochemistry, 43 (2004) 13037-13045)

ATP+Dihydroxyacetone→ADP+Dihydroxyacetone phosphate (EC 2.7.1.29) Phosphoenolpyruvate+Dihydroxyacetone→Pyruvate+Dihydroxyacetone phosphate (EC2.7.1.29)

Dihydroxyacetone kinase can use ATP as a phosphate donor.

The phrase “modified to increase the dihydroxyacetone kinase activity” can mean that the number of the dihydroxyacetone kinase molecules per cell can be increased as compared to that of a wild-type or non-modified strain, or that the activity of the dihydroxyacetone kinase per molecule can be improved as compared to that of a wild-type or non-modified strain. Moreover, when the enzyme activity is undetectable in a wild-type strain, and it is improved to a detectable level, the activity is also considered to be “increased”. Dihydroxyacetone kinase activity per cell can be modified so that it is improved to 150% or more, 200% or more, or even 300% or more, of the activity of a wild-type strain or non-modified strain. Examples of wild-type strains of the microorganism belonging to the family Enterobacteriaceae which can serve as a reference point for comparison include the Escherichia coli MG1655 strain (ATCC No. 47076) and W3110 strain (ATCC No. 27325), Pantoea ananatis AJ13335 strain (FERM BP-6615), and so forth. The dihydroxyacetone kinase activity can be measured by referring to the method of Johnson E. A. (J. Bacteriol., 1984 Oct; 160(1):55-60).

Examples of the gene coding for glycerol dehydrogenase include the gldA gene, including the gldA gene from a microorganism belonging the family Enterobacteriaceae. Examples of the microorganism belonging the family Enterobacteriaceae include Escherichia coli. Examples of the gene of Escherichia coli include, for example, the gldA gene of SEQ ID NO: 1 (complementary strand of the nucleotide numbers 4135955 . . . 4137058 of GenBank Accession No. NC_{—}000913).

Furthermore, the gene coding for glycerol dehydrogenase may be a homologue of the gldA gene. The homologue of the gldA gene can be cloned on the basis of homology to the gene exemplified above from a bacterium of the genus Escherichia, Enterobacter, Klebsiella, Serratia, Erwinia, Yersinia, Shigella, Salmonella, Vibrio, Aeromonas, Bacillus, Staphylococcus, Lactobacillus, Enterococcus, Clostridium, Pseudomonas, Agrobacterium, Citrobacter, Corynebacterium, or the like. Examples of genes that have high homology to the gldA gene of Escherichia coli and encode for glycerol dehydrogenase are mentioned in Table 1.

TABLE 1
Genes showing high homology to gldA gene of Escherichia coli
and coding for glycerol dehydrogenase

| | | | Genbank | SEQ |
|---|---|---|---|---|
| Gene | Microorganism | Description | Accession No. | ID NO |
| gldA | Shigella | Glycerol | YP_405216.1 | 74, 75 |
| | dysenteriae | dehydrogenase, | GI: 82778867 | |
| | Sd197 | (NAD) | | |
| gldA | Salmonella | Similar to E. coli | AE008892.1 | 76, 77 |
| | typhimurium T2 | glycerol | GI: 16422675 | |
| | | dehydrogenasem, | | |
| | | (NAD) | | |
| gldA | Pseudomonas | Glycerol | AF148496.1 | 78, 79 |
| | putida | dehydrogenase | GI: 6552505 | |
| gldA | Bacillus | Glycerol | ZP_01697292.1 | 80, 81 |
| | coagulans | dehydrogenase | GI: 124522908 | |
| | | and related | | |
| | | enzymes | | |

Homology or identity of amino acid sequences and nucleotide sequences can be determined by using, for example, the algorithm BLAST of Karlin and Altschul (Pro. Natl. Acad. Sci. USA, 90, 5873 (1993)) or FASTA of Pearson (Methods Enzymol., 183, 63 (1990)). Programs called BLASTN and BLASTX have been developed on the basis of this algorithm BLAST (refer to www.ncbi.nlm.nih.gov).

As the gene coding for dihydroxyacetone kinase, the dhaKLM genes, dak1, dhaK and dhbK genes can be used. Examples of the gene coding for the enzyme using PEP as a phosphate donor include genes from Escherichia coli, such as the dhaK gene of SEQ ID NO: 34 (complementary strand of the nucleotide numbers 1248991 . . . 1250061 of GenBank Accession No. NC_{—}000913), the dhaL gene of SEQ ID NO: 36 (complementary strand of the nucleotide numbers 1248348 . . . 1248980 of GenBank Accession No. NC_{—}000913), and the dhaM gene of SEQ ID NO: 38 (complementary strand of the nucleotide numbers 1246919 . . . 1248337 of GenBank Accession No. NC_{—}000913).

The gene coding for dihydroxyacetone kinase that uses ATP as a phosphate donor can be used, and it includes the dak1 gene from yeast, the dhbK gene from Agrobacterium bacteria, and the dhaK gene from Citrobacter bacteria. Examples of the dak1 gene from yeast include the dak1 gene of SEQ ID NO: 3 from Saccharomyces cerevisiae (GenBank Accession No NP_{—}013641.1 GI: 6323570), examples of the dhbK gene from Agrobacterium bacteria include the dhbK gene of SEQ ID NO: 5 from Agrobacterium tumefaciens (GenBank Accession No. NP_{—}357070.1 GI: 15891398), and examples of the dhaK gene from Citrobacter bacteria include the dhaK gene of SEQ ID NO: 7 from Citrobacter freundii (GenBank Accession No. U09771).

Furthermore, the gene coding for dihydroxyacetone kinase may be a homologue of the aforementioned genes. Such homologues can be cloned on the basis of homology to the genes exemplified above from a bacterium such as bacteria of the genus Escherichia, Enterobacter, Klebsiella, Serratia, Erwinia, Yersinia, Shigella, Salmonella, Vibrio, Aeromonas, Bacillus, Staphylococcus, Lactobacillus, Enterococcus, Clostridium, Agrobacterium, Citrobacter, and Mycobacterium, yeast such as those of the genus Saccharomyces, Schizosaccharomyces or Pichia, or the like.

In particular, as the gene coding for dihydroxyacetone kinase that uses ATP as a phosphate donor, the following sequences can be used. Genes coding for dihydroxyacetone kinase and showing high homology to the dak1 gene from Saccharomyces cerevisiae are shown in Table 2, dihydroxyacetone kinase genes showing high homology to the dhbK gene from Agrobacterium tumefaciens are shown in Table 3, and dihydroxyacetone kinase genes showing high homology to the dhaK gene from Citrobacter freundii are shown in Table 4.

TABLE 2
Genes coding for dihydroxyacetone kinase and showing high homology to the
dak1 gene from Saccharomyces cerevisiae

| | | | Genbank Accession | SEQ ID |
|---|---|---|---|---|
| Gene | Microorganism | Description | No. | NO |
| T43702 | Schizosaccharomyces | Dihydroxyacetone | gi\|3493578\|gb\| | 40, 41 |
| | pombe | kinase | AAC78808.1\| | |
| AAC27705 | Pichia angusta | Dihydroxyacetone | gi\|3171001\|gb\| | 42, 43 |
| | | kinase | AAC27705.1\| | |
| AAC39490.1 | Pichia pastoris | Dihydroxyacetone | gi\|3287486\|gb\| | 44, 45 |
| | | kinase | AAC39490.1\| | |
| CAG88710.1 | Debaryomyces | Dihydroxyacetone | gi\|49656075\|emb\| | 46, 47 |
| | hansenii CBS767 | kinase | CAG88710.1\| | |

TABLE 3
Genes coding for dihydroxyacetone kinase and showing high homology to the
dhbK gene from Agrobacterium tumefaciens

| | | | Genbank Accession | SEQ ID |
|---|---|---|---|---|
| Gene | Microorganism | Description | No. | NO |
| ABF89849.1 | Myxoccoccus xanthus DK | Dihydroxyacetone | gi\|108464664\| | 58, 59 |
| | 1622 | kinase family protein | gb\|ABF89849.1\| | |
| ABB06761.1 | Burkholderia sp. 383 | Glycerone kinase | gi\|77965380\| | 60, 61 |
| | | | gb\|ABB06761.1\| | |
| | | | Glycerone kinase | |
| | | | [Burkholderia sp. | |
| | | | 383] | |
| ABC38950.1 | Burkholderia | Dihydroxyacetone | gi\|83654887\| | 62, 63 |
| | thailandensis E264 | kinase | gb\|ABC38950.1\| | |
| EAV65448.1 | Burkholderia multivorans | Glycerone kinase | gi\|118658702\| | 64, 65 |
| | ATCC 17616 | | gb\|EAV65448.1\| | |

TABLE 4
Genes coding for dihydroxyacetone kinase and showing high homology to the
dhaK gene from Citrobacter freundii

| | | | Genbank Accession | SEQ ID |
|---|---|---|---|---|
| Gene | Microorganism | Description | No. | NO |
| AAX12907.1 | Escherichia blattae | Dihydroxyacetone | gi\|60099603\| | 48, 49 |
| | | kinase | gb\|AAX12907.1\| | |
| EAV82971.1 | Enterobacter sp. 638 | Dihydroxyacetone | Gi\|118676428\| | 50, 51 |
| | | kinase | gb\|EAV82971.1\| | |
| EAS39398.1 | Psychromonas sp. | Dihydroxyacetone | gi\|90311294\| | 52, 53 |
| | CNPT3 | kinase | gb\|EAS39398.1\| | |
| EAV42339.1 | Stappia aggregata IAM | Dihydroxyacetone | gi\|118435694\| | 54, 55 |
| | 12614 | kinase protein | gb\|EVA42339.1\| | |
| CAK08390.1 | Rhizobium | Putative | gi\|115257295\| | 56, 57 |
| | leguminosarum bv. | dihydroxyacetone | emb\|CAK08390.1\| | |
| | viciae 3841 | kinase | | |

“Fructose-6-phosphate aldolase” is an enzyme that reversibly catalyzes the following reaction that converts dihydroxyacetone into fructose-6-phosphate.

D-Fructose-6-phosphate→Dihydroxyacetone+D-Glyceraldehyde-3-phosphate

The phrase “modified to increase the fructose-6-phosphate aldolase activity” can mean that the number of the fructose-6-phosphate aldolase molecules per cell can be increased as compared to that of a wild-type strain or non-modified strain, or that the activity of the fructose-6-phosphate aldolase per molecule can be improved as compared to that of a wild-type strain or non-modified strain. Fructose-6-phosphate aldolase can be modified so that the activity per cell is improved to 150% or more, 200% or more, or even 300% or more, of the activity observed in a wild-type strain or non-modified strain. Examples of the wild-type strains of the microorganism belonging to the family Enterobacteriaceae which can serve as a reference for comparison include the Escherichia coli MG1655 strain (ATCC No. 47076) and W3110 strain (ATCC No. 27325), Pantoea ananatis AJ13335 strain (FERM BP-6615), and so forth.

Examples of the gene coding for fructose-6-phosphate aldolase include the fsaA gene coding for type I aldolase (SEQ ID NO: 68, the nucleotide numbers 862865 . . . 863527 of GenBank Accession No. NC_{—}000913), and the fsaB gene (talC gene) (SEQ ID NO: 70, complementary strand of the nucleotide numbers 4137069 . . . 4137731 of GenBank Accession No. NC_{—}000913) coding for type II aldolase, which are from Escherichia coli.

Homologues of the gene coding for fructose-6-phosphate aldolase may be a homologue of the aforementioned genes. Such homologues may be cloned on the basis of homology to the fsaA gene or the fsaB gene mentioned above from a bacterium such as bacteria of Escherichia, Enterobacter, Klebsiella, Serratia, Erwinia, Yersinia, Shigella, Salmonella, Vibrio, Aeromonas, Bacillus, Staphylococcus, Lactobacillus, Enterococcus, Clostridium, Pseudomonas, Agrobacterium, Citrobacter, and Corynebacterium.

The fructose-6-phosphate aldolase activity can be measured by using the method of Schurmann M., Sprenger G. A. et al. (J. Biol. Chem., 2001 Apr 6, 276 (14):11055-61).

Homologues of the aforementioned genes be mutant genes derived from other microorganisms, or natural or artificial mutant genes, which show high structural similarity to the aforementioned genes and have a function of improving the glycerol dehydrogenase, dihydroxyacetone kinase or fructose-6-phosphate aldolase activity when they are introduced into a host or amplified. Homologues of glycerol dehydrogenase, dihydroxyacetone kinase, or fructose-6-phosphate aldolase genes mean genes coding for a protein having a homology of 80% or more, 90% or more, 95% or more, 98% or more, or even 99% or more, to the total amino acid sequence of SEQ ID NOs: 2, 4, 6, 8, 69 or 71, or any of the amino acid sequences encoded by the sequences listed in Tables 1 to 4, and having a function of glycerol dehydrogenase, dihydroxyacetone kinase, or fructose-6-phosphate aldolase. The term “homology” may also refer to “identity”. Whether a gene codes for a protein having glycerol dehydrogenase, dihydroxyacetone kinase, or fructose-6-phosphate aldolase activity can be confirmed by expressing the gene in a host cell and examining whether the enzymatic activity is increased as compared to a non-modified strain according to the aforementioned enzymatic activity measurement method. Moreover, whether a gene is a homologue or not can be confirmed by preparing a gene-disrupted strain in which the corresponding wild-type gene is disrupted, introducing the gene into the disrupted strain, and examining whether the gene complements the function of the wild-type gene, for example, whether the enzymatic activity reduced by the gene disruption is restored.

Furthermore, the genes coding for glycerol dehydrogenase, dihydroxyacetone kinase, or fructose-6-phosphate aldolase are not limited to wild-type genes, and they may be conservative variants, i.e., mutant or artificially modified genes coding for a protein having an amino acid sequence of SEQ ID NO: 2, 4, 6, 8, 69 or 71, or any of the amino acid sequences encoded by the sequences listed in Table 1 to 4, which can include substitution, deletion, insertion, addition or the like of one or more amino acid residues at one or more positions so long as the function of the encoded glycerol dehydrogenase, dihydroxyacetone kinase, or fructose-6-phosphate aldolase is not reduced. Although the number of the “one or several” amino acid residues may differ depending on positions in the three-dimensional structure or types of amino acid residues of the protein, it may be specifically 1 to 20, 1 to 10, 1 to 5, or even 1 to 3. These mutations can be conservative substitutions. The conservative substitution is a mutation wherein substitution takes place mutually among Phe, Trp and Tyr, if the substitution site is an aromatic amino acid; among Leu, Ile and Val, if it is a hydrophobic amino acid; between Gln and Asn, if it is a polar amino acid; among Lys, Arg and His, if it is a basic amino acid; between Asp and Glu, if it is an acidic amino acid; and between Ser and Thr, if it is an amino acid having a hydroxyl group. Examples of the conservative substitution include substitution of Ser or Thr for Ala, substitution of Gln, His or Lys for Arg, substitution of Glu, Gln, Lys, His or Asp for Asn, substitution of Asn, Glu or Gln for Asp, substitution of Ser or Ala for Cys, substitution of Asn, Glu, Lys, His, Asp or Arg for Gln, substitution of Gly, Asn, Gln, Lys or Asp for Glu, substitution of Pro for Gly, substitution of Asn, Lys, Gln, Arg or Tyr for His, substitution of Leu, Met, Val or Phe for Ile, substitution of Ile, Met, Val or Phe for Leu, substitution of Asn, Glu, Gln, His or Arg for Lys, substitution of Ile, Leu, Val or Phe for Met, substitution of Trp, Tyr, Met, Ile or Leu for Phe, substitution of Thr or Ala for Ser, substitution of Ser or Ala for Thr, substitution of Phe or Tyr for Trp, substitution of His, Phe or Trp for Tyr, and substitution of Met, Ile or Leu for Val. The aforementioned amino acid substitution, deletion, insertion, addition, inversion or the like may be the result of one or more naturally-occurring mutation(s) due to an individual difference or difference of species (mutant or variant) of a microorganism having the genes coding for glycerol dehydrogenase, dihydroxyacetone kinase, or fructose-6-phosphate aldolase.

The genes coding for glycerol dehydrogenase, dihydroxyacetone kinase, or fructose-6-phosphate aldolase, and conservative variants thereof may also be a DNA which is able to hybridize with a sequence complementary to the nucleotide sequence of SEQ ID NO: 1, 3, 5, 7, 68 or 70, or any of the nucleotide sequences mentioned in Table 1 to 4, or a probe that can be prepared from the nucleotide sequences, under stringent conditions, and codes for a protein having a function of glycerol dehydrogenase, dihydroxyacetone kinase, or fructose-6-phosphate aldolase. The “stringent conditions” are conditions under which a so-called specific hybrid is formed, and a non-specific hybrid is not formed. Although it is difficult to clearly define the condition with numerical values, examples of the stringent condition include those under which highly homologous DNAs hybridize to each other, for example, DNAs not less than 80% homologous, not less than 90% homologous, not less than 95% homologous, not less than 98% homologous, or even not less than 99% homologous, hybridize to each other, and DNAs less homologous than the above do not hybridize to each other, or conditions of washing for typical Southern hybridization, that is, washing once, or 2 or 3 times, at a salt concentration and temperature corresponding to 1×SSC, 0.1% SDS at 60° C., 0.1×SSC, 0.1% SDS at 60° C., or 0.1×SSC, 0.1% SDS at 68° C.

The aforementioned methods can be similarly applied to enhancing the activities of triosephosphate isomerase, fructose bisphosphate aldolase, and fructose-1,6-bisphosphatase described below.

The phrase “intracellular activity of an enzyme increases” can mean when the intracellular activity of the enzyme is increased as compared to a wild-type strain (for example, Escherichia coli W3110 and MG1655 strains), or a parent strain (strain in which intracellular activities of all the enzymes specified are not enhanced), and also include when the cells have the activity that a wild-type or the parent strain does not have.

Examples of methods for increasing the intracellular activity include the following and combinations thereof; however, the methods are not limited to the following. To increase the activities of glycerol dehydrogenase, dihydroxyacetone kinase, and fructose-6-phosphate aldolase, any of the following (1) to (5) can be used in any combination or alone.

(1) Increase in the copy number of a gene coding for each protein by transformation with a vector containing the gene.

(2) Increase in the copy number of a gene coding for each protein by integration of the gene into the chromosome.

(3) Increase in the expression of a gene coding for each protein by modification of an expression control region of the gene.

(4) Increase in expression by modification of a factor which has an effect on expression or the control thereof.

(5) Increase in the enzymatic activity by introduction of a mutation into a coding region of a gene coding for each protein.

(6) Increase in the amount of protein by improvement of the translation efficiency.

Henceforth, the genes coding for enzymes of the DHA pathway may be each referred to as the objective gene.

(1) Increase in the copy number of a gene coding for each protein by transformation with a vector containing the gene

For example, a DNA fragment containing an objective gene can be ligated to a vector, such as a multi-copy vector, which is able to function in the chosen host microorganism to prepare a recombinant DNA. This recombinant DNA can then be introduced into a microorganism to transform it. The objective gene can be obtained by PCR (polymerase chain reaction, refer to White, T. J. et al., Trends Genet., 5, 185 (1989)) using the chromosomal DNA of Escherichia coli, yeast, Citrobacter bacterium, Agrobacterium bacterium or the like as a template. The objective genes from other microorganisms can also be obtained from the chromosomal DNA or a chromosomal DNA library of each microorganism by PCR using, as primers, oligonucleotides prepared based on the known objective gene of the microorganism, or sequence information of the objective gene or the protein of a microorganism of another species, or hybridization using an oligonucleotide prepared based on such sequence information as mentioned above as a probe. A chromosomal DNA can be prepared from a microorganism that serves as a DNA donor by the method of Saito and Miura (refer to Saito H. and Miura K., Biochem. Biophys. Acta, 72, 619 (1963); Experimental Manual for Biotechnology, edited by The Society for Biotechnology, Japan, pp. 97-98, Baifukan Co., Ltd., 1992) or the like.

Then, the objective gene that has been amplified by PCR can be ligated to a vector DNA that is able to function in the cell of a host microorganism to prepare a recombinant DNA. Examples of the vectors that can function in the host microorganism include vectors that are autonomously replicable in cells of the host microorganism.

Examples of vectors that are autonomously replicable in microorganisms belonging to the family Enterobacteriaceae include pUC19, pUC18, pHSG299, pHSG399, pHSG398, pACYC184, (pHSG and pACYC series vectors are available from Takara Bio), RSF1010 (Gene, vol. 75(2), p 271-288, 1989), pBR322, pMW219, pMW119 (pMW series vectors are available form Nippon Gene), pSTV28, pSTV29 (Takara Bio) and so forth. A phage DNA vector can also be used.

To prepare recombinant DNA by ligating any of the genes to the above-mentioned vector, the vector is digested with a restriction enzyme corresponding to the termini of a DNA fragment containing the objective gene. Ligation is generally performed by using a ligase such as T4 DNA ligase. As methods for digesting and ligating DNA, preparation of chromosomal DNA, PCR, preparation of plasmid DNA, transformation, design of oligonucleotides to be used as primers and so forth, methods well known to a person skilled in the art can be employed. These methods are described in Sambrook, J., Fritsch, E. F., and Maniatis, T., “Molecular Cloning A Laboratory Manual, Second Edition”, Cold Sprig Harbor Laboratory Press, (1989), and so forth.

The recombinant DNA prepared as described above may be introduced into a bacterium in accordance with a known transformation method. Examples include electroporation (Canadian Journal of Microbiology, 43, 197 (1997)). It is also possible to use a method of increasing the DNA permeability by treating recipient cells with calcium chloride, which is reported for Escherichia coli K-12 (Mandel, M. and Higa, A., J. Mol. Biol., 53, 159 (1970), or a method of introducing a DNA into a competent cell prepared from a cell at proliferation phase, which is reported for Bacillus subtilis (Duncan, C. H., Wilson, G. A and Young, F. E, Gene, 1, 153 (1977)).

(2) Increase in the copy number of DNA coding for each protein by integration of the gene into a chromosome

Increase of the intracellular activity of each enzyme can be achieved by increasing the copy number of the objective gene by introducing the objective gene into chromosomal DNA of the microorganism. Introduction of the objective gene into the chromosomal DNA of the microorganism can be attained by homologous recombination using a target sequence present on the chromosomal DNA in multiple copies. As sequences present on a chromosomal DNA in multiple copies, a repetitive DNA or an inverted repeat present on the termini of a transposing element can be used. Alternatively, as disclosed in Japanese Patent Laid-open (Kokai) No. 2-109985, the objective gene can be introduced into the chromosomal DNA by inserting the gene into a transposon, and transferring it so that the gene is integrated into the chromosomal DNA. Moreover, it is also possible to introduce an objective gene into a chromosome by using the Red driven integration method (WO2005/010175). An objective gene can also be introduced into a chromosome by transduction using a phage such as P1 phage, or by using a vector for conjugative transfer. Transfer of a gene to a chromosome can be confirmed by Southern hybridization using a part of the gene as a probe. Amplification of the copy number can be confirmed by Southern hybridization using a probe complementary to the objective gene. The gene coding for glycerol dehydrogenase can be amplified by two or more copies, three or more copies, or even five or more copies. The gene coding for dihydroxyacetone kinase can be amplified by two or more copies, three or more copies, or even five or more copies. The gene coding for fructose-6-phosphate aldolase can be amplified by two or more copies, three or more copies, or even five or more copies. When the gene is not native to the chosen host microorganism, any number of copies can be introduced, so long as at least one or more copies are introduced.

(3) Increase in the expression of a gene coding for each protein by modification of an expression control region of the gene

Furthermore, besides increasing the copy number of objective gene mentioned above, increasing the intracellular activity of each enzyme can be achieved by replacing an expression regulatory sequence, such as a promoter, of the gene on a chromosomal DNA or on a plasmid with a stronger promoter by the method described in WO00/18935. Examples of strong promoters include the lac promoter, trp promoter, trc promoter, lambda phage PR promoter, PL promoter, 1pp promoter, T7 promoter, tet promoter, and so forth. In particular, to amplify glycerol dehydrogenase, the tacM promoter (SEQ ID NO: 10) can be used. dhaK, dhaL, and dhaM coding for dihydroxyacetone kinase of Escherichia coli are present as an operon structure, and expression of all three genes can be improved by enhancing the promoter which is located upstream of dhaK.

Moreover, it is also possible to introduce a nucleotide substitution or the like into the promoter region of an objective gene to modify it into a stronger promoter. Methods for evaluating potency of promoters and examples of potent promoters are described in the paper of Goldstein et al. (Prokaryotic promoters in biotechnology, Biotechnol. Annu. Rev., 1995, 1, 105-128), and so forth. Furthermore, it is known that the substitution of several nucleotides in the spacer region between the ribosome binding site (RBS) and the start codon, in particular, in the region immediately upstream of the start codon, significantly affects the translation efficiency of mRNA, and such a region can also be modified. Expression of the objective gene is enhanced by such substitution or modification of promoter.

A promoter located upstream of the objective gene on a genome can be replaced with a stronger promoter by transforming a microorganism belonging to the family Enterobacteriaceae with a DNA containing the stronger promoter amplified by PCR or the like to cause recombination of the stronger promoter and the wild-type promoter on the genome. For such gene substitution utilizing homologous recombination, there can be utilized the method called Red-driven integration (Datsenko, K. A, and Wanner, B. L., Proc. Natl. Acad. Sci. USA, 97:6640-6645 (2000)), a method of using a linear DNA such as a method utilizing the Red driven integration in combination with an excisive system derived from λ phage (Cho, E. H., Gumport, R. I., Gardner, J. F., J. Bacteriol., 184:5200-5203 (2002)) (refer to WO2005/010175), a method of using a plasmid containing a temperature sensitive replication origin (Datsenko, K. A, and Wanner, B. L., Proc. Natl. Acad. Sci. USA, 97:6640-6645 (2000), U.S. Pat. No. 6,303,383, Japanese Patent Laid-open No. 05-007491), and so forth.

(4) Increase in the expression by modification of factor which has an effect on expression or the control thereof

Expression by modification of a factor which effects expression or expression control can be increased by amplifying a gene coding for an activator which increases expression of the genes coding for glycerol dehydrogenase, dihydroxyacetone kinase, or fructose-6-phosphate aldolase, or by deleting or attenuating a gene coding for a regulator which reduces expression of the genes. Examples of an activator of dhaKLM coding for dihydroxyacetone kinase include, for example, dhaR (SEQ ID NO: 66, the nucleotide numbers 1250289 . . . 1252208 of GenBank Accession No. NC_{—}000913), and expression of dhaKLM coding for dihydroxyacetone kinase is increased by a mutation of the dhaR gene (1: EMBO J., 2005 Jan. 26, 24(2):283-93). The expression of dhaKLM coding for dihydroxyacetone kinase is also increased by disruption of the ptsI gene (SEQ ID NO: 86, the nucleotide numbers 2532088 . . . 2533815 of GenBank Accession No. NC_{—}000913) (Microbiology, 147 (2001) 247-253).

(5) Increase in enzymatic activity by introduction of mutation into a coding region of gene coding for each protein

Furthermore, the activities of glycerol dehydrogenase, dihydroxyacetone kinase, and fructose-6-phosphate aldolase can also be increased by introducing a mutation which increases the specific activities of the proteins or improves the substrate specificities of the enzymes into the coding regions of the objective genes.

Such a gene coding for each enzyme having a mutation can be obtained by, for example, modifying the nucleotide sequence of the SEQ ID NO: 1, 3, 5, 7, 68 or 70, or a coding region in any of the nucleotide sequences mentioned in Tables 1 to 4, so that amino acid residues of a specific part of the encoded protein include substitution, deletion, insertion, addition or the like of amino acid residues. Furthermore, conventionally known mutagenizing treatments, such as those described below can be used. For example, by treating the nucleotide sequence of the SEQ ID NO: 1, 3, 5, 7, 68 or 70, any of the nucleotide sequences mentioned in Tables 1 to 4, or a coding region sequence encompassed by any of these, with hydroxylamine or the like in vitro, or by treating a Enterobacteriaceae microorganism containing the gene with ultraviolet radiation or a typical mutagenizing agent, such as N-methyl-N′-nitro-N-nitrosoguanidine (NTG) or ethyl methanesulfonate (EMS), error-prone PCR (Cadwell, R. C., PCR Meth. Appl., 2, 28 (1992)), DNA shuffling (Stemmer, W. P., Nature, 370, 389 (1994)), or StEP-PCR (Zhao, H., Nature Biotechnol., 16, 258 (1998)), a mutation can be artificially introduced into the genes coding for glycerol dehydrogenase, dihydroxyacetone kinase, or fructose-6-phosphate aldolase by gene recombination, resulting in genes coding for highly active glycerol dehydrogenase, dihydroxyacetone kinase, or fructose-6-phosphate aldolase. Whether such mutant genes code for glycerol dehydrogenase, dihydroxyacetone kinase, or fructose-6-phosphate aldolase can be confirmed by, for example, introducing the genes into a microorganism belonging to the family Enterobacteriaceae and having an L-amino acid-producing ability, culturing it in a medium containing glycerol as a carbon source, and confirming whether the L-amino acid-producing ability is improved, or measuring the enzyme activities by the aforementioned methods.

(6) Increase in the amount of protein by improving the translation efficiency

The amount of protein can be increased by improving the translation efficiency by increasing the tRNA corresponding to codons less frequently used in the host, or by modifying the objective gene so that it has optimal codons according to the frequency of use of codons in the host (Gene 85, 109-114 (1989), Biochemistry, 31, 2598-2608 (1992), J. Bacteriol., 175, 716-722 (1993), Protein Expression and Purification, 50, 49-57 (2006)). The The increase in the amount of the objective protein as compared to a non-modified strain or wild-type strain can be confirmed by, for example, detection by Western blotting using antibodies (Molecular Cloning (Cold Spring Harbor Laboratory Press, Cold spring Harbor (USA), 2001)).

The microorganism can be modified to increase glycerol uptake activity, in addition to enhancing glycerol dehydrogenase, dihydroxyacetone kinase, and fructose-6-phosphate aldolase activities. The glycerol uptake activity can mean the activity for incorporating glycerol into the cytoplasm, and a glycerol facilitator, which is a membrane protein, is involved. Examples of the gene coding for the glycerol facilitator include, for example, the glpF gene of Escherichia coli (SEQ ID NO: 16, complementary strand of the nucleotide numbers 4115268 . . . 4116113 of GenBank Accession No. NC_{—}000913).

The gene coding for the glycerol facilitator may be a DNA which hybridizes with a complementary sequence of the nucleotide sequence of SEQ ID NO: 16, or a probe which can be prepared from the complementary sequence under stringent conditions, and codes for a protein having glycerol uptake activity. Examples also include a DNA coding for the protein of SEQ ID NO: 17. The protein can have homology of 80% or more, 90% or more, 95% or more, 98% or more, or even 99% or more, to the total amino acid sequence of SEQ ID NO: 17, so long as it increases the glycerol uptake ability in a microorganism belonging to the family Enterobacteriaceae, when it is introduced into the microorganism.

Moreover, the gene may be a DNA coding for a protein having an amino acid sequence of SEQ ID NO: 17, but which can include substitution, deletion, insertion, addition or the like of one or several amino acid residues, so long as the glycerol uptake activity is not reduced. The activity can be increased by a method similar to the aforementioned methods for enhancing glycerol dehydrogenase, dihydroxyacetone kinase, or fructose-6-phosphate aldolase activity.

The glycerol uptake activity can be measured by using the transport assay method using a membrane protein (Voegele, R. T., Sweet, G. D., and Boos, W. J., Bacteriol., 175:1087-1094 (1993)).

The microorganism can be modified to increase the activities of one or more enzymes such as triosephosphate isomerase, fructose bisphosphate aldolase, and fructose-1,6-bisphosphatase, in addition to enhancing glycerol dehydrogenase, dihydroxyacetone kinase, and fructose-6-phosphate aldolase activities, and enhancing the glycerol uptake activity.

Triosephosphate isomerase catalyzes the reaction which reversibly converts dihydroxyacetone phosphate into glyceraldehyde-3-phosphate (EC:5.3.1.1).

Dihydroxyacetone phosphate→D-Glyceraldehyde-3-phosphate

The phrase “modified to increase the triosephosphate isomerase activity” can mean that the number of the triosephosphate isomerase molecules per cell can be increased as compared to that of a wild-type strain or non-modified strain, or that the activity of the triosephosphate isomerase per molecule can be improved as compared to that of a wild-type strain or non-modified strain. The triosephosphate isomerase can be modified so that the activity per cell is improved to 150% or more, 200% or more, or even 300% or more, of the activity of a wild-type strain or non-modified strain. Examples of wild-type strains of the microorganism belonging to the family Enterobacteriaceae which can serve as a reference for comparison include the Escherichia coli MG1655 strain (ATCC No. 47076) and W3110 strain (ATCC No. 27325), Pantoea ananatis AJ13335 strain (FERM BP-6615), and so forth.

Examples of the gene coding for triosephosphate isomerase include the tpiA gene derived from Escherichia coli (SEQ ID NO: 18, complementary strand of the nucleotide numbers 4108763 . . . 4109530 of GenBank Accession No. NC_{—}000913).

The gene coding for triosephosphate isomerase may be a DNA which hybridizes with a complementary sequence of the nucleotide sequence of SEQ ID NO: 18, or a probe which can be prepared from the complementary sequence, under stringent conditions, and codes for a protein having the triosephosphate isomerase activity. Examples also include a DNA coding for the protein of SEQ ID NO: 19. The protein can have homology of 80% or more, 90% or more, 95% or more, 98% or more, or even 99% or more, to the total amino acid sequence of SEQ ID NO: 19, so long as it shows increased triosephosphate isomerase activity in a microorganism belonging to the family Enterobacteriaceae, when it is introduced into the microorganism.

Moreover, the gene may be a DNA coding for a protein having an amino acid sequence of SEQ ID NO: 19, but which can include substitution, deletion, insertion, addition or the like of one or several amino acid residues, so long as the triosephosphate isomerase activity is not reduced.

The triosephosphate isomerase activity can be measured by using the method of Andersen and Cooper (FEBS Lett., 4, 19-20 (1969)). The activity can be increased by methods similar to the aforementioned methods for enhancing glycerol dehydrogenase, dihydroxyacetone kinase, and fructose-6-phosphate aldolase activities.

“Fructose bisphosphate aldolase” reversibly catalyzes the following reaction which converts dihydroxyacetone phosphate and glyceroaldehyde-3-phosphate into D-fructose-1,6-bisphosphate (EC:4.1.2.13).

Dihydroxyacetone phosphate (Glycerone phosphate)+D-Glyceraldehyde-3-phosphate→D-Fructose-1,6-bisphosphate

The phrase “modified to increase the fructose bisphosphate aldolase activity” can mean that the number of the fructose bisphosphate aldolase molecules per cell can be increased as compared to that of a wild-type strain or non-modified strain, or that the activity of the fructose bisphosphate aldolase per molecule can be improved as compared to that of a wild-type strain or non-modified strain. The fructose bisphosphate aldolase can be modified so that the activity per cell is improved to 150% or more, 200% or more, or even 300% or more, of the activity of a wild-type strain or non-modified strain. Examples of wild-type strains of the microorganism belonging to the family Enterobacteriaceae which can serve as a reference for comparison include the Escherichia coli MG1655 (ATCC No. 47076) and W3110 strains (ATCC No. 27325), Pantoea ananatis AJ13335 strain (FERM BP-6615), and so forth.

Examples of the gene coding for fructose bisphosphate aldolase include the fbaA gene derived from Escherichia coli (SEQ ID NO: 20, complementary strand of the nucleotide numbers 3068187 . . . 3069266 of GenBank Accession No. NC_{—}000913) and the fbaB gene derived from Escherichia coli (SEQ ID NO: 72, complementary strand of the nucleotide numbers 2175534 . . . 2176586 of GenBank Accession No. NC_{—}000913).

The gene coding for fructose bisphosphate aldolase can be a DNA which hybridizes with a complementary sequence of the nucleotide sequence of SEQ ID NO: 20 or 72, or a probe which can be prepared from the complementary sequence, under stringent conditions, and codes for a protein having the fructose bisphosphate aldolase activity. Examples also include a DNA coding for the protein of SEQ ID NO: 21 or 73. The protein can have homology of 80% or more, 90% or more, 95% or more, 98% or more, or even 99% or more, to the total amino acid sequence of SEQ ID NO: 21, so long as it shows increased fructose bisphosphate aldolase activity in a microorganism belonging to the family Enterobacteriaceae, when it is introduced into the microorganism.

Moreover, the gene may be a DNA coding for a protein having an amino acid sequence of SEQ ID NO: 21 or 73, but which can include substitution, deletion, insertion, addition or the like of one or several amino acid residues, so long as the fructose bisphosphate aldolase activity is not reduced.

The fructose bisphosphate aldolase activity can be measured by using the method of Richard & Rutter (J. Biol. Chem., 236, 3177-3184). The activity can be increased by methods similar to the aforementioned methods for enhancing glycerol dehydrogenase, dihydroxyacetone kinase, and fructose-6-phosphate aldolase activities.

“Fructose-1,6-bisphosphatase” reversibly catalyzes the following reaction that converts D-fructose-1,6-bisphosphate into D-fructose-6-phosphate (EC:3.1.3.11).

D-Fructose-1,6-bisphosphate+H₂O→D-Fructose-6-phosphate+Phosphate

The phrase “being modified to increase the fructose-1,6-bisphosphatase activity” can mean that the number of the fructose-1,6-bisphosphatase molecules per cell can be increased as compared to that of a wild-type strain or non-modified strain, or that the activity of the fructose-1,6-bisphosphatase per molecule is improved as compared to that of a wild-type strain or non-modified strain. The fructose-1,6-bisphosphatase can be modified so that the activity per cell is improved to 150% or more, 200% or more, or even 300% or more, of the activity of a wild-type strain or non-modified strain. Examples of wild-type strains of the microorganism belonging to the family Enterobacteriaceae which can serve as a reference for comparison include the Escherichia coli MG1655 (ATCC No. 47076) and W3110 strains (ATCC No. 27325), Pantoea ananatis AJ13335 strain (FERM BP-6615), and so forth.

Examples of the gene coding for fructose-1,6-bisphosphatase include the glpX gene (SEQ ID NO: 22, complementary strand of the nucleotide numbers 4112592 . . . 4113602 of GenBank Accession No. NC_{—}000913), the fbp gene (SEQ ID NO: 82, the nucleotide numbers 4452634 . . . 4453632 of GenBank Accession No. NC_{—}000913), and the ybhA gene (SEQ ID NO: 84, the nucleotide numbers 796836 . . . 7976554 of GenBank Accession No. NC_{—}000913), which are derived from Escherichia coli.

The gene coding for the fructose-1,6-bisphosphatase may be a DNA which hybridizes with a complementary sequence of the nucleotide sequence of SEQ ID NO: 22, 82 or 84, or a probe which can be prepared from the complementary sequence, under stringent conditions, and codes for a protein having the fructose-1,6-bisphosphatase activity. Examples also include a DNA coding for the protein of SEQ ID NO: 23, 83 or 85. The protein can be a protein showing a homology of 80% or more, preferably 90% or more, more preferably 95% or more, still more preferably 98% or more, particularly preferably 99% or more, to the total amino acid sequence of SEQ ID NO: 23, 83 or 85, so long as it shows increased fructose-1,6-bisphosphatase activity in a microorganism belonging to the family Enterobacteriaceae, when it is introduced into the microorganism.

Moreover, the gene may be a DNA coding for a protein having an amino acid sequence of SEQ ID NO: 23, 83 or 85, but which can include substitution, deletion, insertion, addition or the like of one or several amino acid residues, so long as the fructose-1,6-bisphosphatase activity is not reduced.

The fructose-1,6-bisphosphatase activity can be measured by using the method of Nakajima et al. (Protein Nucleic Enzyme, 22, 1585-1589). The activity can be increased by methods similar to the aforementioned methods for enhancing glycerol dehydrogenase and dihydroxyacetone kinase.

The microorganism can be modified to reduce the activities of glycerol kinase and/or membrane-binding type glycerol-3-phosphate dehydrogenase, in addition to enhancing the activities of glycerol dehydrogenase, dihydroxyacetone kinase, and fructose-6-phosphate aldolase. In addition, the glycerol uptake activity can be enhanced, as well as the activities of one or more kinds of of triosephosphate isomerase, fructose bisphosphate aldolase, and fructose-1,6-bisphosphatase.

“Glycerol kinase” reversibly catalyzes the following reaction that generates glycerol-3-phosphate and ADP from glycerol and ATP (EC2.7.1.30)

ATP+Glycerol→ADP+sn-Glycerol-3-phosphate

The phrase “modified to reduce the glycerol kinase activity” can mean that the number of the glycerol kinase molecules per cell is decreased as compared to that of a wild-type strain or non-modified strain, or that the activity of the glycerol kinase per molecule is reduced as compared to that of a wild-type strain or non-modified strain. The glycerol kinase is modified so that the activity per cell is reduced to 70% or less, 50% or less, 30% or less, or even 20% or less, of the activity of a wild-type strain or non-modified strain, and the enzymatic activity may be completely eliminated. The enzymatic activity can be decreased by reducing the expression of the gene coding for the enzyme. Reducing the expression of the gene can include reducing the amount of the mRNA transcribed from the gene and reducing the translation amount of the mRNA.

The production of the enzyme protein molecule can be completely eliminated, or the activity per enzyme protein molecule can be reduced or deleted by disrupting the gene coding for the enzyme. Examples of wild-type strains of the microorganism belonging to the family Enterobacteriaceae which can serve as a reference for comparison include the Escherichia coli MG1655 strain (ATCC No. 47076) and W3110 strain (ATCC No. 27325), Pantoea ananatis AJ13335 strain (FERM BP-6615), and so forth.

Examples of the gene coding for glycerol kinase include the glpK gene (SEQ ID NO: 24, complementary strand of the nucleotide numbers 4113737 . . . 4115245 of GenBank Accession No. NC_{—}000913) derived from Escherichia coli. The enzymatic activity of glycerol kinase can be measured by the method of Thorner & Paulus (The Enzymes, 3rd ed., 8, 487-508).

“Membrane-binding type glycerol-3-phosphate dehydrogenase” catalyzes the oxidation reaction converting glycerol-3-phosphate to dihydroxyacetone phosphate, and reversibly catalyzes the following reaction.

sn-Glycerol-3P+Ubiquinone→Dihydroxyacetone-P+Ubiquinol (EC:1.1.99.5)

The phrase “modified to reduce the membrane-binding type glycerol-3-phosphate dehydrogenase activity” can mean that the number of the membrane-binding type glycerol-3-phosphate dehydrogenase molecules per cell is decreased as compared to that of a wild-type strain or non-modified strain, or that the activity of the membrane-binding type glycerol-3-phosphate dehydrogenase per molecule is reduced as compared to that of a wild-type strain or non-modified strain. The membrane-binding type glycerol-3-phosphate dehydrogenase can be modified so that the activity per cell is reduced to 70% or less, 50% or less, or even 30% or less, of the activity of a wild-type strain or non-modified strain, and the enzymatic activity may be completely eliminated. The enzymatic activity can be decreased by reducing the expression of the gene coding for the enzyme. Examples of wild-type strains of the microorganism belonging to the family Enterobacteriaceae which can serve as a reference for comparison include the Escherichia coli MG1655 strain (ATCC No. 47076) and W3110 strain (ATCC No. 27325), Pantoea ananatis AJ13335 strain (FERM BP-6615), and so forth.

The membrane-binding type glycerol-3-phosphate dehydrogenase is encoded by the glpABC operon and the glpD gene, and examples of the glpA gene of Escherichia coli include the sequence of SEQ ID NO: 26 (the nucleotide numbers 2350669 . . . 2352297 of GenBank Accession No. NC_{—}000913), examples of the glpB gene of Escherichia coli include the sequence of SEQ ID NO: 28 (the nucleotide numbers 2352287 . . . 2353546 of GenBank Accession No. NC_{—}000913), examples of the glpC gene of Escherichia coli include the sequence of SEQ ID NO: 30 (the nucleotide numbers 2353543 . . . 2354733 of GenBank Accession No. NC_{—}000913), and examples of the glpD gene of Escherichia coli include the sequence of SEQ ID NO: 32 (the nucleotide numbers 3560036 . . . 3561541 of GenBank Accession No. NC_{—}000913).

Activity of an objective enzyme such as glycerol kinase and glycerol-3-phosphate dehydrogenase mentioned above can be reduced by

(1) reduction or deletion of the enzymatic activity by introducing a mutation into the coding region of the gene coding for the objective enzyme, or

(2) reduction or deletion of the enzymatic activity by modifying an expression control sequence of the gene coding for the objective enzyme.

(1) Reduction or deletion of the enzymatic activity by introducing a mutation into the coding region of gene coding for the objective enzyme

A mutation can be introduced into the coding region of the gene coding for an objective enzyme by introducing a mutation for an amino acid substitution (missense mutation), a stop codon (nonsense mutation), or a frame shift mutation which adds or deletes one or two nucleotides into a region of the objective gene coding for the enzyme on a chromosome by genetic recombination (Journal of Biological Chemistry, 272:8611-8617 (1997); Proceedings of the National Academy of Sciences, USA, 95 5511-5515 (1998); Journal of Biological Chemistry, 266, 20833-20839 (1991)). A part or the entire gene in the coding region can be deleted. Specifically, a mutation can be introduced into the DNA of SEQ ID NO: 24, 26, 28, 30 or 32, or deleting a part or all of such DNA.

The enzymatic activity can also be reduced or deleted by constructing a gene coding for a mutant enzyme in which the coding region is deleted or into which a mutation is introduced, and substituting the mutated gene for the normal gene on the chromosome by homologous recombination or the like, or by introducing a transposon or IS factor into the gene.

To introduce mutations as described above into a gene by genetic recombination, for example, the following methods can be used. A partial sequence of an objective gene can be modified to prepare a mutant gene designed so that it does not produce an enzyme that functions normally, and transforming a microorganism belonging to the family Enterobacteriaceae with a DNA containing the gene to cause recombination of the mutant gene and the corresponding gene on the chromosome. In this way, the objective gene on the chromosome can be replaced with the mutant gene. For such gene substitution utilizing homologous recombination, thefollowing methods can be used: Red-driven integration (Datsenko, K. A, and Wanner, B. L., Proc. Natl. Acad. Sci. USA, 97:6640-6645 (2000)), using a linear DNA in a method utilizing the Red driven integration in combination with an excisive system derived from λ phage (Cho, E. H., Gumport, R. I., Gardner, J. F., J. Bacteriol., 184:5200-5203 (2002)) (refer to WO2005/010175), using a plasmid containing a temperature-sensitive replication origin (Datsenko, K. A, and Wanner, B. L., Proc. Natl. Acad. Sci. USA, 97:6640-6645 (2000), U.S. Pat. No. 6,303,383, Japanese Patent Laid-open No. 05-007491), and so forth. Moreover, such site-specific mutagenesis based on gene substitution utilizing homologous recombination as described above can also be performed by using a plasmid that is not able to replicate in a host. Moreover, the enzymatic activity can also be reduced or deleted by introducing a mutation into the coding region of the objective gene using a known mutation treatment, such as X-ray or ultraviolet irradiation, or by using a mutation agent such as N-methyl-N′-nitro-N-nitrosoguanidine.

(2) Reduction or deletion of enzymatic activity by modifying an expression control sequence of the gene coding for the objective enzyme

Enzymatic activity by modification of an expression control sequence of the gene coding for the objective enzyme can also be reduced or deleted by reducing the expression by introducing a mutation into an expression control sequence, such as a promoter or a SD sequence, on a chromosomal DNA, by amplifying the gene coding for a regulator which reduces expression of the gene, or by deleting or attenuating the gene coding for an activator which improves expression of the gene. Methods for evaluating the potency of promoters and examples of potent promoters are described in the paper of Goldstein et al. (Prokaryotic promoters in biotechnology, Biotechnol. Annu. Rev., 1995, 1, 105-128), and so forth. Furthermore, it is known that by replacing several nucleotides in the spacer region between the ribosome binding site (RBS) and the start codon, in particular, in the region immediately upstream from the start codon, the translation efficiency of mRNA can be significantly affected, and such a region can also be modified. In particular, the glpA, B, and C genes make up an operon structure, and therefore the expression of the entire operon can be reduced by introducing a mutation into an expression control region such as the promoter region located upstream of glpA.

In the bacterium, in order to further enhance the glycerol assimilation ability, expression of the glpR gene (EP 1715056) may be attenuated, or expression of the glycerol metabolism genes such as glpE, glpG, glpQ, glpT, and dhK genes (EP 1715055 A, International Patent Publication WO2007/013695) may be enhanced.

<3> Microorganisms and Methods for Imparting L-Amino Acid-Producing Ability

A bacterium belonging to the family Enterobacteriaceae that is able to produce L-amino acids, which can metabolize glycerol as a carbon source, can be used as a parent strain. A bacterium obtained by imparting the aforementioned properties suitable for glycerol metabolism to such a parent strain can also be used. Alternatively, the L-amino acid-producing ability may be imparted by the method described below to a bacterium already having the aforementioned properties.

The family Enterobacteriaceae encompasses bacteria belonging to the genera of Escherichia, Enterobacter, Erwinia, Klebsiella, Pantoea, Photorhabdus, Providencia, Salmonella, Serratia, Shigella, Morganella, Yersinia, and so forth. In particular, bacteria classified into the family Enterobacteriaceae according to the taxonomy used by the NCBI (National Center for Biotechnology Information) database (www.ncbi.nlm.nih.gov/Taxonomy/Browser/wwwtax.cgi?id=91347) are preferred.

The expression “a bacterium belonging to the genus Escherichia” can mean that the bacterium is classified into the genus Escherichia according to classification known to a person skilled in the art of microbiology, although the bacterium is not particularly limited. Examples of the bacterium belonging to the genus Escherichia include, but are not limited to, Escherichia coli (E. coli).

The bacterium belonging to the genus Escherichia is not particularly limited. However, examples include, for example, the bacteria of the phyletic groups described in the work of Bachmann et al., Table 1 (Bachmann, B. J., 1996, pp. 2460-2488, In F. D. Neidhardt (ed.), Escherichia coli and Salmonella: Cellular and Molecular Biology/Second Edition, American Society for Microbiology Press, Washington, D.C.). Specific examples include Escherichia coli W3110 (ATCC 27325), Escherichia coli MG1655 (ATCC 47076), and so forth, which are derived from the prototype wild-type strain, K12 strain.

These strains are available from, for example, American Type Culture Collection (Address: 12301 Parklawn Drive, Rockville, Md. 20852, United States of America). That is, accession numbers are given to each of the strains, and the strains can be ordered by using these numbers. The accession numbers of the strains are listed in the catalogue of the American Type Culture Collection.

The expression of a bacterium belonging to the genus Pantoea means that the bacterium is classified into the genus Pantoea according to classification known to a person skilled in the art of microbiology. Some strains of Enterobacter agglomerans have been recently re-classified into Pantoea agglomerans, Pantoea ananatis, Pantoea stewartii or the like based on the nucleotide sequence analysis of 16S rRNA etc. (Int. J. Syst. Bacteriol., 43, 162-173 (1993)). Bacteria belonging to the genus Pantoea can encompass such bacteria which have been re-classified into the genus Pantoea as described above.

A bacterium having an L-amino acid-producing ability (the ability to produce an L-amino acid) means a bacterium that can produce and secrete an L-amino acid in a medium when it is cultured in the medium. It can also mean a bacterium which can accumulate an objective L-amino acid in the medium in an amount not less than 50 g/L, not less than 75 g/L, or even not less than 100 g/L. The “L-amino acid” can encompass L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine. L-Threonine and L-lysine are particular examples.

Any of the L-amino acid-producing bacteria reported to date can be used as a parent strain, so long as the chosen strain can assimilate glycerol, or an ability to assimilate glycerol can be imparted. Hereafter, L-amino acid-producing bacteria are described for each L-amino acid.

L-Threonine-producing bacteria

Examples of L-threonine-producing bacteria and parent strains which can be used to derive such bacteria include, but are not limited to, strains belonging to the genus Escherichia, such as E. coli TDH-6/pVIC40 (VKPM B-3996) (U.S. Pat. No. 5,175,107, U.S. Pat. No. 5,705,371), E. coli 472T23/pYN7 (ATCC 98081) (U.S. Pat. No. 5,631,157), E. coli NRRL-21593 (U.S. Pat. No. 5,939,307), E. coli FERM BP-3756 (U.S. Pat. No. 5,474,918), E. coli FERM BP-3519 and FERM BP-3520 (U.S. Pat. No. 5,376,538), E. coli MG442 (Gusyatiner et al., Genetika (in Russian), 14, 947-956 (1978)), E. coli VL643 and VL2055 (EP 1149911 A) and so forth.

The strain TDH-6 is deficient in the thrC gene, as well as being sucrose-assimilative, and the ilvA gene has a leaky mutation. This strain also has a mutation in the rhtA gene, which imparts resistance to high concentration of threonine or homoserine. The B-3996 strain harbors the plasmid pVIC40 obtained by inserting a thrA*BC operon containing a mutant thrA gene into a RSF1010-derived vector. This mutant thrA gene encodes aspartokinase homoserine dehydrogenase I which is substantially desensitized to feedback inhibition by threonine. The B-3996 strain was deposited on Nov. 19, 1987 at the All-Union Scientific Center of Antibiotics (Nagatinskaya Street 3-A, 117105 Moscow, Russia) under the accession number RIA 1867. The strain was also deposited at the Russian National Collection of Industrial Microorganisms (VKPM) on Apr. 7, 1987 under the accession number VKPM B-3996.

E. coli VKPM B-5318 (EP 0593792 B) can also be used as an L-threonine-producing bacterium or a parent strain for deriving it. The B-5318 strain is prototrophic with regard to isoleucine, and in this strain, a temperature-sensitive lambda-phage Cl repressor and PR promoter replace the regulatory region of the threonine operon in the plasmid pVIC40. The strain VKPM B-5318 was deposited at the Russian National Collection of Industrial Microorganisms (VKPM) (1 Dorozhny proezd., 1 Moscow 117545, Russia) on May 3, 1990 under the accession number of VKPM B-5318.

The bacterium can be additionally modified to increase expression of one or more of the following genes:
the mutant thrA gene which codes for aspartokinase-homoserine dehydrogenase I resistant to feed back inhibition by threonine;
the thrB gene which codes for homoserine kinase;
the thrC gene which codes for threonine synthase;
the rhtA gene which codes for a putative transmembrane protein;
the asd gene which codes for aspartate-f3-semialdehyde dehydrogenase; and
the aspC gene which codes for aspartate aminotransferase (aspartate transaminase).

The thrA gene which encodes aspartokinase-homoserine dehydrogenase I of Escherichia coli has been elucidated (nucleotide numbers 337 to 2799, GenBank accession NC_{—}000913.2, gi: 49175990). The thrA gene is located between the thrL and thrB genes on the chromosome of E. coli K-12. The thrB gene which encodes homoserine kinase of Escherichia coli has been elucidated (nucleotide numbers 2801 to 3733, GenBank accession NC_{—}000913.2, gi: 49175990). The thrB gene is located between the thrA and thrC genes on the chromosome of E. coli K-12. The thrC gene which encodes threonine synthase of Escherichia coli has been elucidated (nucleotide numbers 3734 to 5020, GenBank accession NC_{—}000913.2, gi: 49175990). The thrC gene is located between the thrB gene and the yaaX open reading frame on the chromosome of E. coli K-12. All three of these genes function as a single threonine operon. To increase expression of the threonine operon, the attenuator region that affects the transcription can be removed from the operon (WO2005/049808, WO2003/097839).

The mutant thrA gene which codes for aspartokinase-homoserine dehydrogenase I resistant to feed back inhibition by threonine as well as the thrB and thrC genes can be obtained as one operon from the well-known plasmid pVIC40 which is present in the threonine-producing E. coli strain VKPM B-3996. The plasmid pVIC40 is described in detail in U.S. Pat. No. 5,705,371.

The rhtA gene is present at 18 min on the E. coli chromosome close to the glnHPQ operon, which encodes components of the glutamine transport system. The rhtA gene is identical to ORF1 (ybiF gene, nucleotide numbers 764 to 1651, GenBank accession number AAA218541, gi:440181), and is located between the pexB and ompX genes. The unit expressing a protein encoded by the ORF1 has been designated rhtA gene (rht: resistance to homoserine and threonine). It has also been revealed that the rhtA23 mutation is an A-for-G substitution at position −1 with respect to the ATG start codon (ABSTRACTS of the 17th International Congress of Biochemistry and Molecular Biology in conjugation with Annual Meeting of the American Society for Biochemistry and Molecular Biology, San Francisco, Calif. Aug. 24-29, 1997, abstract No. 457; EP 1013765 A).

The asd gene of E. coli has already been elucidated (nucleotide numbers 3572511 to 3571408, GenBank Accession NC_{—}000913.1, gi:16131307), and can be obtained by PCR (refer to White, T. J., Arnheim, N., and Erlich, H. A., Trends Genet., 5, 185-189 (1989)) utilizing primers prepared based on the nucleotide sequence of the gene. The asd genes of other microorganisms can be obtained in a similar manner.

The aspC gene of E. coli has also already been elucidated (nucleotide numbers 983742 to 984932, GenBank Accession NC_{—}000913.1, gi:16128895), and can be obtained by PCR. The aspC genes of other microorganisms can be obtained in a similar manner.

L-Lysine-Producing Bacteria

Examples of L-lysine-producing bacteria belonging to the genus Escherichia include mutants having resistance to an L-lysine analogue. L-Lysine analogues inhibit growth of bacteria belonging to the genus Escherichia, but this inhibition is fully or partially desensitized when L-lysine is present in a medium. Examples of the L-lysine analogue include, but are not limited to, oxalysine, lysine hydroxamate, S-(2-aminoethyl)-L-cysteine (AEC), γ-methyllysine, α-chlorocaprolactam and so forth. Mutants having resistance to these lysine analogues can be obtained by subjecting bacteria belonging to the genus Escherichia to a conventional artificial mutagenesis treatment. Specific examples of bacterial strains useful for producing L-lysine include Escherichia coli AJ11442 (FERM BP-1543, NRRL B-12185; see U.S. Pat. No. 4,346,170) and Escherichia coli VL611. In these microorganisms, feedback inhibition of aspartokinase by L-lysine is desensitized.

The WC196 strain can be used as an L-lysine-producing bacterium of Escherichia coli. This bacterial strain was bred from the W3110 strain, which was derived from Escherichia coli K-12, by replacing the lysC gene on the chromosome of the W3110 strain with a mutant lysC gene encoding a mutant aspartokinase III in which threonine at position 352 had been replaced with isoleucine, resulting in desensitization of the feedback inhibition thereof by L-lysine (U.S. Pat. No. 5,661,012), and then conferring AEC resistance to the resulting strain. This strain was designated Escherichia coli AJ13069 and was deposited at the National Institute of Bioscience and Human-Technology, Agency of Industrial Science and Technology (currently National Institute of Advanced Industrial Science and Technology, International Patent Organism Depositary, Central 6, 1-1, Higashi 1-Chome, Tsukuba-shi, Ibaraki-ken, 305-8566, Japan) on Dec. 6, 1994 and assigned an accession number of FERM P-14690. Then, it was converted to an international deposit under the provisions of the Budapest Treaty on Sep. 29, 1995, and assigned an accession number of FERM BP-5252 (U.S. Pat. No. 5,827,698).

Examples of L-lysine-producing bacteria and parent strains which can be used to derive such bacteria also include strains in which expression of one or more genes encoding an L-lysine biosynthetic enzyme is increased. Examples of such genes include, but are not limited to, dihydrodipicolinate synthase gene (dapA), aspartokinase gene (lysC), dihydrodipicolinate reductase gene (dapB), diaminopimelate decarboxylase gene (lysA), diaminopimelate dehydrogenase gene (ddh) (U.S. Pat. No. 6,040,160), phosphoenolpyrvate carboxylase gene (ppc), aspartate semialdehyde dehydrogenease gene (asd), and aspartase gene (aspA) (EP 1253195 A). In addition, the parent strains can have an increased level of expression of the gene involved in energy efficiency (cyo) (EP 1170376 A), the gene encoding nicotinamide nucleotide transhydrogenase (pntAB) (U.S. Pat. No. 5,830,716), the ybjE gene (WO2005/073390), the gene coding for glutamate dehydrogenase (gdhA, Gene, 23:199-209 (1983)), or combinations thereof. Abbreviations of the genes are indicated in the parentheses.

It is known that wild-type dihydrodipicolinate synthetase derived from Escherichia coli is subject to feedback inhibition by L-lysine, while wild-type aspartokinase derived from Escherichia coli is subject to suppression and feedback inhibition by L-lysine. Therefore, when the dapA and lysC genes are used, these genes can be mutant genes coding the enzymes that are not subject to the feedback inhibition by L-lysine.

Examples of DNA encoding a mutant dihydrodipicolinate synthetase desensitized to feedback inhibition by L-lysine include a DNA encoding a protein that has the amino acid sequence of the enzyme in which the histidine at position 118 is replaced by tyrosine. Examples of DNA encoding a mutant aspartokinase desensitized to feedback inhibition by L-lysine include a DNA encoding an AKIII having the amino acid sequence in which the threonine at position 352, the glycine at position 323, and the methionine at position 318 are replaced by isoleucine, asparagine and isoleucine, respectively (for these mutants, see U.S. Pat. No. 5,661,012 and U.S. Pat. No. 6,040,160). Such mutant DNAs can be obtained by site-specific mutagenesis using PCR or the like.

Wide host-range plasmids RSFD80, pCAB1, and pCABD2 are known as plasmids containing a mutant dapA gene encoding a mutant dihydrodipicolinate synthetase and a mutant lysC gene encoding a mutant aspartokinase (U.S. Pat. No. 6,040,160). Escherichia coli JM109 strain transformed with RSFD80 was named AJ12396 (U.S. Pat. No. 6,040,160), the strain was deposited at the National Institute of Bioscience and Human-Technology, Agency of Industrial Science and Technology, Ministry of International Trade and Industry (currently, International Patent Organism Depositary, National Institute of Advanced Industrial Science and Technology) on Oct. 28, 1993 and assigned an accession number of FERM P-13936, and the deposit was then converted to an international deposit under the provisions of Budapest Treaty on Nov. 1, 1994 and assigned an accession number of FERM BP-4859. RSFD80 can be obtained from the AJ12396 strain by a known method.

Examples of L-lysine-producing bacteria and parent strains which can be used to derive such bacteria also include strains having decreased or no activity of an enzyme that catalyzes a reaction that generates a compound other than L-lysine by branching off from the biosynthetic pathway of L-lysine. Examples of such enzymes include homoserine dehydrogenase, lysine decarboxylase (U.S. Pat. No. 5,827,698), and the malic enzyme (WO2005/010175). In order to reduce or eliminate the lysine decarboxylase activity, the expression of both the cadA gene and ldcC gene coding for lysine decarboxylase can be reduced (International Patent Publication WO2006/038695).

Examples of cadA gene- and ldcC gene-disrupted strain include the Escherichia coli WC196LC (WC196AcadAAldc) strain. The WC196LC strain, which was designated AJ110692, was deposited at National Institute of Advanced Industrial Science and Technology, International Patent Organism Depositary (Central 6, 1-1, Higashi 1-Chome, Tsukuba-shi, Ibaraki-ken, 305-8566, Japan) on Oct. 7, 2008 as an international deposit, and assigned an accession number of FERM BP-11027.

L-Cysteine-Producing Bacteria

Examples of L-cysteine-producing bacteria and parent strains which can be used to derive such bacteria include, but are not limited to, strains belonging to the genus Escherichia, such as E. coli JM15 which is transformed with different cysE alleles coding for feedback-resistant serine acetyltransferases (U.S. Pat. No. 6,218,168, Russian Patent Application No. 2003121601); E. coli W3110 having over-expressed genes which encode proteins suitable for secreting substances toxic for cells (U.S. Pat. No. 5,972,663); E. coli strains having lowered cysteine desulfohydrase activity (Japanese Patent Laid-open No. 11-155571); and E. coli W3110 with increased activity of a positive transcriptional regulator for cysteine regulon encoded by the cysB gene (WO01/27307).

L-Leucine-Producing Bacteria

Examples of L-leucine-producing bacteria and parent strains which can be used to derive such bacteria include, but are not limited to, strains belonging to the genus Escherichia, such as E. coli strains resistant to leucine (for example, the strain 57 (VKPM B-7386, U.S. Pat. No. 6,124,121)) or leucine analogues including β-2-thienylalanine, 3-hydroxyleucine, 4-azaleucine and 5,5,5-trifluoroleucine (Japanese Patent Publication (Kokoku) No. 62-34397 and Japanese Patent Laid-open No. 8-70879); E. coli strains obtained by a gene engineering method described in WO96/06926; and E. coli H-9068 (Japanese Patent Laid-open No. 8-70879).

The bacterium can be improved by enhancing expression of one or more genes involved in L-leucine biosynthesis. Such genes include the genes of the leuABCD operon, a typical example of which is a mutant leuA gene coding for isopropyl malate synthase desensitized to feedback inhibition by L-leucine (U.S. Pat. No. 6,403,342). In addition, the bacterium can be improved by increasing expression of one or more genes coding for proteins which excrete L-amino acid from bacterial cells. Examples of such genes include the b2682 and b2683 genes (ygaZH genes) (EP 1239041 A2).

L-Histidine-Producing Bacteria

Examples of L-histidine-producing bacteria and parent strains which can be used to derive such bacteria include, but are not limited to, strains belonging to the genus Escherichia, such as E. coli strain 24 (VKPM B-5945, RU 2003677); E. coli strain 80 (VKPM B-7270, RU 2119536); E. coli NRRL B-12116 to B12121 (U.S. Pat. No. 4,388,405); E. coli H-9342 (FERM BP-6675) and H-9343 (FERM BP-6676) (U.S. Pat. No. 6,344,347); E. coli H-9341 (FERM BP-6674) (EP 1085087); and E. coli AI80/pFM201 (U.S. Pat. No. 6,258,554).

Examples of L-histidine-producing bacteria and parent strains which can be used to derive such bacteria also include strains in which expression of one or more genes encoding an L-histidine biosynthetic enzyme is increased. Examples of such genes include ATP phosphoribosyl transferase gene (hisG), phosphoribosyl AMP cyclohydrolase gene (hisI), phosphoribosyl-ATP pyrophosphohydrolase gene (hisI), phosphoribosylformimino-5-aminoimidazole carboxamide ribotide isomerase gene (hisA), amidotransferase gene (hisH), histidinol phosphate aminotransferase gene (hisC), histidinol phosphatase gene (hisB), histidinol dehydrogenase gene (hisD), and so forth.

It is known that the L-histidine biosynthetic enzymes encoded by hisG and hisBHAFI are inhibited by L-histidine, and therefore L-histidine-producing ability can also be efficiently enhanced by introducing a mutation which confers resistance to the feedback inhibition into the ATP phosphoribosyl transferase gene (hisG) (Russian Patent Nos. 2003677 and 2119536).

Specific examples of strains having L-histidine-producing ability include E. coli FERM-P 5038 and 5048 which are introduced with a vector carrying a DNA encoding an L-histidine biosynthetic enzyme (Japanese Patent Laid-open No. 56-005099), E. coli strains introduced with a gene for amino acid-export (EP 1016710 A), E. coli 80 strain imparted with sulfaguanidine, DL-1,2,4-triazole-3-alanine, and streptomycin resistance (VKPM B-7270, Russian Patent No. 2119536), and so forth.

L-Glutamic Acid-Producing Bacteria

Examples of L-glutamic acid-producing bacteria and parent strains which can be used to derive such bacteria include, but are not limited to, strains belonging to the genus Escherichia, such as E. coil VL334thrC⁺ (EP 1172433). E. coli VL334 (VKPM B-1641) is an L-isoleucine and L-threonine auxotrophic strain having mutations in thrC and ilvA genes (U.S. Pat. No. 4,278,765). A wild-type allele of the thrC gene was transferred by the method of general transduction using a bacteriophage P1 grown on the wild-type E. coli K12 strain (VKPM B-7) cells. As a result, an L-isoleucine auxotrophic L-glutamic acid-producing strain VL334thrC⁺ (VKPM B-8961) was obtained.

Examples of L-glutamic acid-producing bacteria and parent strains which can be used to derive such bacteria include, but are not limited to, strains in which expression of one or more genes encoding an L-glutamic acid biosynthetic enzyme is increased. Examples of such genes include genes encoding glutamate dehydrogenase (gdhA), glutamine synthetase (glnA), glutamate synthetase (gltAB), isocitrate dehydrogenase (icdA), aconitate hydratase (acnA, acnB), citrate synthase (OA), phosphoenolpyruvate carboxylase (ppc), pyruvate dehydrogenase (aceEF, lpdA), pyruvate kinase (pykA, pykF), phosphoenolpyruvate synthase (ppsA), enolase (eno), phosphoglyceromutase (pgmA, pgml), phosphoglycerate kinase (pgk), glyceraldehyde-3-phophate dehydrogenase (gapA), triose phosphate isomerase (tpiA), fructose bisphosphate aldolase (fbp), phosphofructokinase (pfkA, pfkB), glucose phosphate isomerase (pgi), and so forth.

Examples of strains modified to increase expression of the citrate synthetase gene, the phosphoenolpyruvate carboxylase gene, and/or the glutamate dehydrogenase gene include those disclosed in EP 1078989 A, EP 955368 A and EP 952221 A.

Examples of L-glutamic acid-producing bacteria and parent strains which can be used to derive such bacteria also include strains having decreased or no activity of an enzyme that catalyzes synthesis of a compound other than L-glutamic acid by branching off from the L-glutamic acid biosynthesis pathway. Examples of such enzymes include isocitrate lyase (aceA), a-ketoglutarate dehydrogenase (sucA), phosphotransacetylase (pta), acetate kinase (ack), acetohydroxy acid synthase (ilvG), acetolactate synthase (ilvI), formate acetyltransferase (pfl), lactate dehydrogenase (ldh), glutamate decarboxylase (gadAB), γ-glutamyl transferase (ggt), γ-glutamylcysteine synthetase (gshA), γ-glutamylputrescine synthetase (ycjK), and so forth. Bacteria belonging to the genus Escherichia deficient in α-ketoglutarate dehydrogenase activity or having reduced α-ketoglutarate dehydrogenase activity and methods for obtaining them are described in U.S. Pat. Nos. 5,378,616 and 5,573,945.

Specific examples of such strains include the followings:

E. coli W3110sucA::Km^{r}

E. coli AJ12624 (FERM BP-3853)

E. coli AJ12628 (FERM BP-3854)

E. coli AJ12949 (FERM BP-4881)

E. coli W3110sucA::Km^{r} is a strain obtained by disrupting the a-ketoglutarate dehydrogenase gene (hereinafter also referred to as “sucA gene”) of E. coli W3110. This strain is completely deficient in α-ketoglutarate dehydrogenase.

Other examples of L-glutamic acid-producing bacteria include those which belong to the genus Escherichia and have resistance to an aspartic acid antimetabolite. These strains may also be deficient in a-ketoglutarate dehydrogenase, and examples include, for example, E. coli AJ13199 (FERM BP-5807) (U.S. Pat. No. 5,908,768), FFRM P-12379) which additionally has a lowered L-glutamic acid decomposing ability (U.S. Pat. No. 5,393,671); AJ13138 (FERM BP-5565) (U.S. Pat. No. 6,110,714), and so forth.

Examples of L-glutamic acid-producing bacteria include bacteria belonging to the genus Pantoea which are deficient in α-ketoglutarate dehydrogenase activity or have a decreased α-ketoglutarate dehydrogenase activity, and they can be obtained as described above. Such strains include Pantoea ananatis AJ13356 (U.S. Pat. No. 6,331,419). Pantoea ananatis AJ13356 was deposited at the National Institute of Bioscience and Human-Technology, Agency of Industrial Science and Technology (currently, National Institute of Advanced Industrial Science and Technology, International Patent Organism Depositary, Central 6, 1-1, Higashi 1-Chome, Tsukuba-shi, Ibaraki-ken, 305-8566, Japan) on Feb. 19, 1998 under an accession number of FERM P-16645. It was then converted to an international deposit under the provisions of Budapest Treaty on Jan. 11, 1999 and assigned an accession number of FERM BP-6616. Pantoea ananatis AJ13356 is deficient in α-ketoglutarate dehydrogenase activity as a result of disruption of the aKGDH-E1 subunit gene (sucA). This strain was identified as Enterobacter agglomerans when it was isolated and deposited as the Enterobacter agglomerans AJ13356. However, it was recently re-classified as Pantoea ananatis on the basis of nucleotide sequencing of 16S rRNA and so forth. Although AJ13356 was deposited at the aforementioned depository as Enterobacter agglomerans, it is described as Pantoea ananatis in this specification.

L-Phenylalanine-Producing Bacteria

Examples of L-phenylalanine-producing bacteria and parent strains which can be used to derive such bacteria include, but are not limited to, strains belonging to the genus Escherichia, such as E. coil AJ12739 (tyrA::Tn10, tyrR) (VKPM B-8197); E. coli HW1089 (ATCC 55371) harboring the mutant pheA34 gene (U.S. Pat. No. 5,354,672); E. coli MWEC101-b (KR 8903681); E. coli NRRL B-12141, NRRL B-12145, NRRL B-12146 and NRRL B-12147 (U.S. Pat. No. 4,407,952). As parent strains, E. coli K-12 [W3110 (tyrA)/pPHAB] (FERM BP-3566), E. coli K-12 [W3110 (tyrA)/pPHAD] (FERM BP-12659), E. coli K-12 [W3110 (tyrA)/pPHATerm] (FERM BP-12662) and E. coli K-12 [W3110 (tyrA)/pBR-aroG4, pACMAB] named as AJ12604 (FERM BP-3579) may also be used (EP 488424 B1). Furthermore, L-phenylalanine-producing bacteria belonging to the genus Escherichia with an enhanced activity of the protein encoded by the yedA gene or the yddG gene can also be used (U.S. Patent Published Application Nos. 2003/0148473 A1 and 2003/0157667 A1).

L-Tryptophan-Producing Bacteria

Examples of tryptophan-producing bacteria and parent strains which can be used to derive such bacteria include, but are not limited to, strains belonging to the genus Escherichia, such as E. coli JP4735/pMU3028 (DSM10122) and JP6015/pMU91 (DSM10123), which are deficient in the tryptophanyl-tRNA synthetase encoded by a mutant trpS gene (U.S. Pat. No. 5,756,345); E. coli SV164 (pGH5) having a serA allele encoding phosphoglycerate dehydrogenase free from feedback inhibition by serine and a trpE allele encoding anthranilate synthase free from feedback inhibition by tryptophan (U.S. Pat. No. 6,180,373); E. coli AGX17(pGX44) (NRRL B-12263) and AGX6(pGX50)aroP (NRRL B-12264) deficient in tryptophanase (U.S. Pat. No. 4,371,614); and E. coli AGX17/pGX50,pACKG4-pps in which phosphoenolpyruvate-producing ability is enhanced (WO97/08333, U.S. Pat. No. 6,319,696). L-Tryptophan-producing bacteria belonging to the genus Escherichia with an enhanced activity of the protein encoded by the yedA gene or the yddG gene may also be used (U.S. Patent Published Application Nos. 2003/0148473 A1 and 2003/0157667 A1).

Examples of L-tryptophan-producing bacteria and parent strains which can be used to derive such bacteria also include strains in which one or more activities of the enzymes selected from anthranilate synthase (trpE), phosphoglycerate dehydrogenase (serA), and tryptophan synthase (trpAB) are increased. Anthranilate synthase and phosphoglycerate dehydrogenase both are subject to feedback inhibition by L-tryptophan and L-serine, and therefore a mutation can be introduced into these enzymes that results in desensitizing them to the feedback inhibition. Specific examples of strains having such a mutation include E. coli SV164 that harbors desensitized anthranilate synthase and a transformant strain obtained by introducing the plasmid pGH5 (WO94/08031), which contains a mutant serA gene encoding feedback inhibition-desensitized phosphoglycerate dehydrogenase, into the E. coli SV164.

Examples of L-tryptophan-producing bacteria and parent strains which can be used to derive such bacteria also include strains into which the tryptophan operon containing a gene encoding inhibition-desensitized anthranilate synthase is introduced (Japanese Patent Laid-open Nos. 57-71397, 62-244382, U.S. Pat. No. 4,371,614). Moreover, L-tryptophan-producing ability may be imparted by increasing expression of a gene which encodes tryptophan synthase in the tryptophan operon (trpBA). The tryptophan synthase consists of α and β subunits which are encoded by the trpA and trpB genes, respectively. In addition, L-tryptophan-producing ability may also be improved by increasing expression of the isocitrate lyase-malate synthase operon (WO2005/103275).

L-Proline-Producing Bacteria

Examples of L-proline-producing bacteria and parent strains which can be used to derive such bacteria include, but are not limited to, strains belonging to the genus Escherichia, such as E. coli 702ilvA (VKPM B-8012), which is deficient in the ilvA gene and is able to produce L-proline (EP 1172433).

The bacterium may be improved by increasing expression of one or more genes involved in L-proline biosynthesis. Such genes include the proB gene coding for glutamate kinase desensitized to feedback inhibition by L-proline (DE 3127361). In addition, the bacterium can be improved by increasing expression of one or more genes coding for proteins responsible for the secretion of L-amino acid from the bacterial cells. Examples of such genes include the b2682 and b2683 genes (ygaZH genes) (EP 1239041 A2).

Examples of bacteria belonging to the genus Escherichia and having L-proline-producing ability include the following E. coli strains: NRRL B-12403 and NRRL B-12404 (British Patent No. 2075056), VKPM B-8012 (Russian Patent Application No. 2000124295), plasmid mutants described in German Patent No. 3127361, plasmid mutants described by Bloom F. R. et al. (The 15th Miami winter symposium, 1983, p. 34), and so forth.

L-Arginine-Producing Bacteria

Examples of L-arginine-producing bacteria and parent strains which can be used to derive such bacteria include, but are not limited to, strains belonging to the genus Escherichia, such as E. coli strain 237 (VKPM B-7925) (U.S. Patent Published Application No. 2002/058315A1) and its derivative strains harboring a mutant N-acetylglutamate synthase (Russian Patent Application No. 2001112869), E. coli strain 382 (VKPM B-7926) (EP 1170358 A1), and an arginine-producing strain into which argA gene encoding N-acetylglutamate synthetase is introduced (EP 1170361 A1).

Examples of L-arginine-producing bacteria and parent strains which can be used to derive such bacteria also include strains in which expression of one or more genes encoding an L-arginine biosynthetic enzyme is increased. Examples of such genes include N-acetylglutamyl phosphate reductase gene (argC), ornithine acetyl transferase gene (argJ), N-acetylglutamate kinase gene (argB), acetylornithine transaminase gene (argD), ornithine carbamoyl transferase gene (argF), argininosuccinic acid synthetase gene (argG), argininosuccinic acid lyase gene (argH), and carbamoyl phosphate synthetase gene (carAB).

L-Valine-Producing Bacteria

Example of L-valine-producing bacteria and parent strains which can be used to derive such bacteria include, but are not limited to, strains which have been modified to overexpress the ilvGMEDA operon (U.S. Pat. No. 5,998,178). It is desirable to remove the region of the ilvGMEDA operon that is required for attenuation so that expression of the operon is not attenuated by produced L-valine. Furthermore, the ilvA gene in the operon can be disrupted so that threonine deaminase activity is decreased.

Examples of L-valine-producing bacteria and parent strains which can be used to derive such bacteria also include mutants having a mutation of amino-acyl t-RNA synthetase (U.S. Pat. No. 5,658,766). For example, E. coli VL1970, which has a mutation in the ileS gene encoding isoleucine tRNA synthetase, can be used. E. coli VL1970 was deposited at the Russian National Collection of Industrial Microorganisms (VKPM) (1 Dorozhny Proezd, 1 Moscow 117545, Russia) on Jun. 24, 1988 under the accession number of VKPM B-4411.

Furthermore, mutants requiring lipoic acid for growth and/or lacking H⁺-ATPase (WO96/06926) can also be used as the parent strains.

L-Isoleucine-Producing Bacteria

Examples of L-isoleucine-producing bacteria and parent strains which can be used to derive such bacteria include, but are not limited to, mutants having resistance to 6-dimethylaminopurine (Japanese Patent Laid-open No. 5-304969 A), mutants having resistance to an isoleucine analogue such as thiaisoleucine and isoleucine hydroxamate, and such mutants further having resistance to DL-ethionine and/or arginine hydroxamate (Japanese Patent Laid-open No. 5-130882). In addition, recombinant strains transformed with genes encoding proteins involved in L-isoleucine biosynthesis, such as threonine deaminase and acetohydroxate synthase, can also be used as the parent strains (Japanese Patent Laid-open No. 2-458, FR 0356739, and U.S. Pat. No. 5,998,178).

L-Asparagine-Producing Bacteria

L-Asparagine is produced by transferring an amino group to aspartic acid (Boehlein, S. K., Richards, N. G. J., & Schuster, S. M. (1994a), J. Biol. Chem., 269, 7450-7457). Therefore, examples of L-asparagine-producing bacteria belonging to the genus Escherichia include L-asparatic acid-producing Escherichia coli strains in which asparagine synthetase is enhanced.

### EXAMPLES

Hereafter, the present invention will be more specifically explained with reference to the following non-limiting examples.

### Reference Example 1

Construction of L-lysine-producing bacterium with enhanced fructose-6-phosphate aldolase, glycerol dehydrogenase, and dihydroxyacetone kinase activities (WO2008/102861)

<1-1> Construction of Plasmid for dak1 Gene Expression

The total nucleotide sequence of Saccharomyces cerevisiae chromosome has already been determined (Science, 25 October, 274(5287), 546-567 (1996)). On the basis of this nucleotide sequence of the dak1 gene as reported, the synthetic oligonucleotide of SEQ ID NO: 14 was prepared as a 5′ primer, and the synthetic oligonucleotide of SEQ ID NO: 15 was prepared as a 3′ primer. PCR was performed by using these synthetic oligonucleotides and the chromosomal DNA of the Saccharomyces cerevisiae JCM7255 strain as a template. The PCR product was purified and ligated with the vector pMW 119 (Takara Bio) digested with HindIII and SalI to construct a dak1 expression plasmid, pMW-dak1. The JCM7255 strain is stored in the independent administrative agency, RIKEN, “Japan Collection of Microorganisms”, 2-1, Hirosawa, Wako-shi, Saitama-ken.

<1-2> Construction of a Strain with Improved Glycerol Dehydrogenase Activity

A strain containing the glycerol dehydrogenase gene (PtacMgldA::Cm) having the structure shown in SEQ ID NO: 11 was constructed from the WC196ΔcadAΔldcC strain (refer to International Patent Publication WO2006/038695). For the construction of the strain having this structure, the DNA of SEQ ID NO: 9 (PCR product) was used. In the sequence of the DNA of SEQ ID NO: 9, the nucleotide numbers 1 to 172 is the attR sequence of λ phage, the nucleotide numbers 324 to 983 is a chloramphenicol resistance gene (cat), the nucleotide numbers 1540 to 1653 is the attL sequence of λ phage, and the nucleotide numbers 1654 to 1733 is the tacM promoter.

The tacM promoter (SEQ ID NO: 10) can be constructed by replacing the TTGACA sequence of the tac promoter (Gene, 25 (2-3), 167-178 (1983)) at the −35 region with TTCACA. The sequence of SEQ ID NO: 9 can be constructed by referring to the construction of pMW118-attL-Cm-attR (WO2005/010175).

The DNA of SEQ ID NO: 9 was used as the template and was amplified by PCR using the primers of SEQ ID NOS: 12 and 13, and this amplification product was inserted into the chromosome of the WC196ΔcadAΔldcC strain by the λ-RED method (WO2005/010175) to construct a strain in which the promoter sequence upstream of the gldA was replaced. In this way, a strain with improved glycerol dehydrogenase activity, the WC196ΔcadAΔldcCPtacMgldA::Cm strain, was obtained.

<1-3> Construction of L-Lysine-Producing Bacterium with Enhanced Fructose-6-Phosphate Aldolase and Glycerol Dehydrogenase Activities

A strain containing the fructose-6-phosphate aldolase gene and the glycerol dehydrogenase gene (PtacM fsaB-gldA::Cm sequence) having the structure shown in SEQ ID NO: 88 was constructed from the WC196ΔcadAΔldcC strain. For the construction of the strain having this structure, the DNA of SEQ ID NO: 9 (PCR product) was used. In the sequence of SEQ ID NO: 9, the nucleotide numbers 1 to 172 is the attR sequence of λ phage, the nucleotide numbers 324 to 983 is a chloramphenicol resistance gene (cat), the nucleotide numbers 1540 to 1653 is the attL sequence of λ phage, and the nucleotide numbers 1654 to 1733 is the tacM promoter.

The tacM promoter (SEQ ID NO: 10) can be constructed by replacing the TTGACA sequence of the tac promoter (Gene, 25 (2-3), 167-178 (1983)) at the −35 region with TTCACA. The DNA of SEQ ID NO: 9 can be constructed by referring to the construction of pMW118-attL-Cm-attR (WO2005/010175).

The DNA of SEQ ID NO: 9 was used as the template and was amplified by PCR using the primers of SEQ ID NOS: 89 and 90, and this amplification product was inserted into the chromosome of the WC196ΔcadAΔldcC strain (refer to International Publication WO2006/038695) by the λ-RED method (WO2005/010175) to construct a strain in which the promoter sequence upstream of the fsaB-gldA operon was replaced. In this way, a strain with improved fructose-6-phosphate aldolase and glycerol dehydrogenase activities, the WC196ΔcadAΔldcCPtacM fsaB-gldA::Cm strain, was obtained.

<1-4> Construction of L-Lysine-Producing Bacteria with Enhanced Fructose-6-Phosphate Aldolase, Glycerol Dehydrogenase, and Dihydroxyacetone Kinase Activities

The WC196ΔcadAΔldcC strain (refer to International Publication WO2006/038695), the WC196ΔcadAΔldcCPtacMgldA::Cm strain, and the WC196ΔcadAΔldcCPtacM fsaB-gldA::Cm strain were transformed with the plasmid pCABD2 for Lys production carrying dapA, dapB and lysC genes (International Publication WO01/53459) in a conventional manner to obtain the WC196ΔcadAΔldcC/pCABD2 strain, the WC196ΔcadAΔldcCPtacMg1dA::Cm/pCABD2 strain, and the WC196ΔcadAΔldcCPtacM fsaB-gldA::Cm/pCABD2 strain. Furthermore, the WC196ΔcadAΔldcC/pCABD2 strain, the WC196ΔcadAΔldcCPtacMgldA::Cm/pCABD2 strain, and the WC196ΔcadAΔldcCPtacM fsaB-gldA::Cm/pCABD2 strain were transformed with the dak1 expression plasmid pMW-dak1 in a conventional manner to obtain the WC196ΔcadAΔldcC/pCABD2,pMW-dak1 strain, the WC196ΔcadAΔldcCPtacMgldA::Cm/pCABD2,pMW-dak1 strain and the WC196ΔcadAΔldcCPtacM fsaB-gldA::Cm/pCABD2,pMW-dak1 strain.

These strains were each cultured in L medium containing 20 mg/L of streptomycin, or 20 mg/L of streptomycin and 50 mg/L of ampicillin at 37° C. until the final OD600 became about 0.6, then a 40% glycerol solution in a volume equal to the culture medium was added to each culture medium, and the mixture was stirred, then divided into appropriate volumes, and stored at −80° C. These are called glycerol stocks.

### Reference Example 2

Evaluation of L-lysine-producing bacteria with enhanced fructose-6-phosphate aldolase, glycerol dehydrogenase, and dihydroxyacetone kinase activities

The aforementioned glycerol stocks of the strains were thawed, 100 μL of each stock was uniformly applied to an L plate containing 20 mg/L of streptomycin, or 20 mg/L of streptomycin and 50 mg/L of ampicillin, and the culture was performed at 37° C. for 24 hours. The obtained cells on the plate were suspended in 1 ml of physiological saline, the suspension was diluted 101 times, and the diluted suspension was inoculated in a volume corresponding to 50/n, wherein n is the absorbance of the diluted suspension at 600 nm, into 20 mL of a fermentation medium containing 20 mg/L of streptomycin, or 20 mg/L of streptomycin and 50 mg/L of ampicillin in a 500-mL Sakaguchi flask. The culture was performed at 37° C. for 48 hours on a reciprocally shaking culture machine. After completion of the culture, the amount of lysine which had accumulated in the medium was measured by a known method (Biotec Analyzer AS210, SAKURA SEIKI).

The composition of the fermentation medium is shown below (unit: g/L):

| | |
|---|---|
| Glycerol | 40 |
| (NH₄)₂SO₄ | 24 |
| K₂HPO₄ | 1.0 |
| MgSO₄•7H₂O | 1.0 |
| FeSO₄•7H₂O | 0.01 |
| MnSO₄•5H₂O | 0.01 |
| Yeast extract | 2.0 |

To a final volume of 1 L

The medium was adjusted to pH 7.0 with KOH, and autoclaved at 115° C. for 10 minutes, provided that glycerol and MgSO₄.7H₂O were separately sterilized, and 30 g/L of CaCO₃ of Japanese Pharmacopoeia subjected to hot air sterilization at 180° C. for 2 hours was added.

As antibiotics, 20 mg/L of streptomycin, or 20 mg/L of streptomycin and 50 mg/L of ampicillin were added. The culture was performed at a temperature of 37° C. with stirring at 115 rpm for 48 hours.

As a result, the WC196ΔcadAΔldcCPtacMgldA::Cm/pCABD2,pMW-dak1 strain in which glycerol dehydrogenase and dihydroxyacetone kinase using ATP as a phosphate donor were both enhanced, and the WC196ΔcadAΔldcCPtacM fsaB-gldA::Cm/pCABD2,pMW-dak1 strain in which fructose-6-phosphate aldolase was further enhanced, both produced larger amounts of L-lysine as compared to the non-modified strain.

### Example 1

The aforementioned glycerol stock of the WC196ΔcadAΔldcCPtacM fsaB-gldA::Cm/pCABD2,pMW-dak1 strain was thawed, 100 μL of the stock was uniformly applied to an L plate containing 20 mg/L of streptomycin and 50 mg/L of ampicillin, and the culture was performed at 37° C. for 18 hours. The obtained cells on the plate were suspended in 5 ml of physiological saline, the suspension was diluted 101 times, and the diluted suspension was inoculated in a volume corresponding to 146/n, wherein n is absorbance of the diluted suspension at 620 nm, into 300 mL of a fermentation medium (A medium) containing 20 mg/L of streptomycin and 50 mg/L of ampicillin in a 1-L volume jar fermenter, and a seed culture was performed at 37° C. and pH 6.7 for 15 hours.

After the completion of the culture, 15% the seed culture medium relative to the A medium volumne was inoculated to 300 mL of the A medium containing 20 mg/L of streptomycin and 50 mg/L of ampicillin in a 1-L volume jar fermenter, and the culture was started at 37° C. and pH 6.7 and continued for 20 hours. After 8 hours of the culture, the glycerol concentration in the medium was controlled to be 0 to 5 g/L, 10 to 15 g/L, or 15 to 20 g/L by adding B medium dropwise. The glycerol concentration was measured with BF-5 produced by Oji Scientific Instruments.

Composition of L plate medium:

| | |
|---|---|
| Tryptone | 10 g/L |
| Yeast extract | 5 g/L |
| NaCl | 10 g/L |
| Agar | 20 g/L |

Composition of A medium:

| | | |
|---|---|---|
| Glycerol | 40.0 | g/L |
| Mameno*¹⁾ | 11 | mL/L |
| Ammonium sulfate | 8.5 | g/L |
| KH₂PO₄ | 1.0 | g/L |
| MgSO₄•7H₂O | 1.0 | g/L |
| FeSO₄•7H₂O | 30 | mg/L |
| MnSO₄•4H₂O | 30 | mg/L |
| Threonine | 0.21 | g/L |
| Methionine | 0.13 | g/L |
| Betaine*²⁾ | 2 | g/L |

*¹⁾Soy bean hydrolysate (total nitrogen: 0.35 g/L)
*²⁾N,N,N-Trimethylglycine

Composition of B medium:

| | |
|---|---|
| Glycerol | 450 g/L |
| Threonine | 0.84 g/L |
| KH₂PO₄ | 3.37 g/L |
| Ammonium sulfate | 106 g/L |

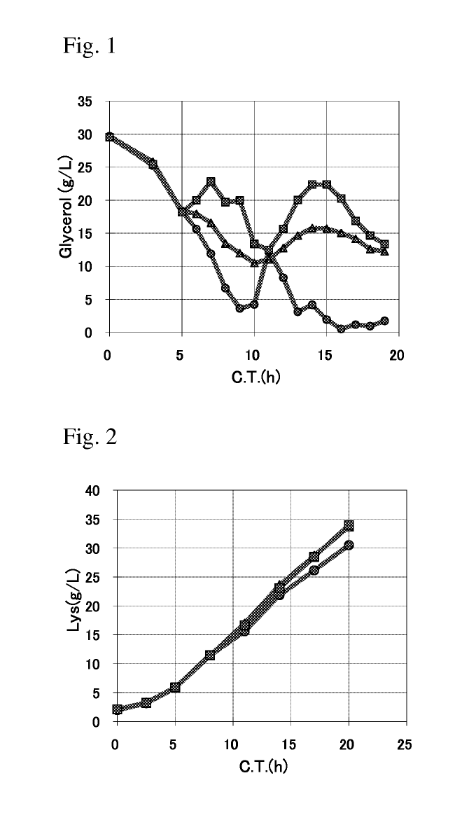

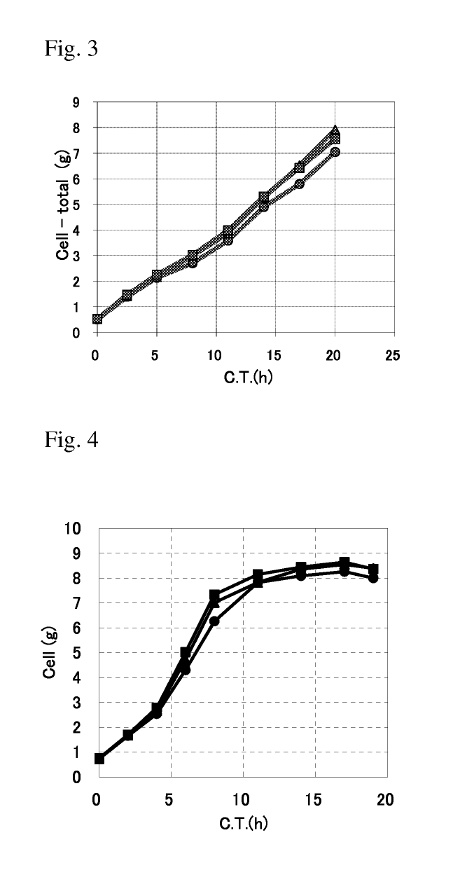

The results of the culture are shown in Table 5, and the change in the glycerol concentration over time and the change of amount of L-lysine which accumulated over time are shown in FIGS. 1 to 3. When the glycerol concentration was controlled to be 10 to 15 g/L, or 15 to 20 g/L, the accumulation and productivity of L-lysine were improved, as compared to when the glycerol concentration was controlled to be 0 to 5 g/L.

TABLE 5
Results of culture

| | | | | Consumed | Lys |
|---|---|---|---|---|---|
| | | Lys | Lys | glycerol | production |
| Controlled | | yield | productivity | conc. | amount |
| concentration | | (%) | (g/L/h) | (g/fermenter) | (g/L) |
| 0-5 (4.1 ± 3.5) | g/L | 35.3 | 1.58 | 26.8 | 30.5 |
| 10-15 (13.3 ± 1.8) | g/L | 35.1 | 1.74 | 31.7 | 33.7 |
| 15-20 (17.6 ± 3.6) | g/L | 36.2 | 1.75 | 31.0 | 33.9 |

*) Average glycerol concentrations after 8 hours and deviations thereof are in the parentheses in the table.

### Reference Example 3

### Construction of L-Threonine-Producing Bacterium with Enhanced Glycerol Dehydrogenase and Dihydroxyacetone Kinase Activities

<3-1> Construction of a Strain with Improved Glycerol Dehydrogenase Activity

A strain containing the gldA gene having the structure shown in SEQ ID NO: 91 was constructed from the E. coli VKPM B-5318 strain (EP 0593792 B). For the construction of this strain, the DNA of SEQ ID NO: 92 (PCR product) was used. In the DNA of SEQ ID NO: 92, the nucleotide numbers 1 to 72 is the attR sequence of λ phage, the nucleotide numbers 324 to 983 is the chloramphenicol resistance gene (cat), the nucleotide numbers 1540 to 1653 is the attL sequences of λ phage, and the nucleotide numbers 1654 to 1733 is the tacM2 promoter.

The tacM2 promoter is a constitutive promoter that can be constructed by replacing the TTGACA sequence of the tac promoter (Gene, 25 (2-3), 167-178 (1983)) at the −35 region with TGTACA (Molecular Biology, 39 (5) 719-726 (2005)). The DNA of SEQ ID NO: 92 can be constructed by referring to the construction of pMW118-attL-Cm-attR (WO2005/010175).

The DNA of SEQ ID NO: 92 was used as the template and was amplified by PCR using the primers of SEQ ID NOS: 93 and 94, and this amplification product was inserted into chromosome of the B-5318 strain (VKPM B-5318) by the λ-RED method (WO2005/010175) to obtain a strain in which the promoter sequence upstream of the gldA was replaced. In this way, a strain with improved glycerol dehydrogenase activity, B5318PtacM2gldA::Cm strain, was obtained.

<3-2> Construction of L-Threonine-Producing Bacterium with Enhanced Glycerol Dehydrogenase and Dihydroxyacetone Kinase Activities

The B5318PtacM2gldA::Cm strain was transformed with the dak1 expression plasmid pMW-dak1 in a conventional manner to obtain B5318PtacM2gldA::Cm/pMW-dak1 strain.

### Example 2

A glycerol stock of the aforementioned strain was thawed, 100 μL of the stock was uniformly applied to an L plate containing 20 mg/L of streptomycin and 50 mg/L of ampicillin, and the culture was performed at 37° C. for 18 hours. The cells that appeared on the plate were suspended in 5 ml of physiological saline, the suspension was diluted 101 times, and the diluted suspension was inoculated in a volume (V) obtained by dividing a constant, 146, with absorbance of the diluted suspension at 620 nm (n) (V=146/n), into 300 mL of a fermentation medium (C medium) containing 20 mg/L of streptomycin and 50 mg/L of ampicillin in a 1-L volume jar fermenter, and the seed culture was performed at 37° C. and pH 6.7 for 15 hours.

After completion of the culture, 15% of the seed culture medium relative to the C medium was inoculated into 300 mL of C medium containing 20 mg/L of streptomycin and 50 mg/L of ampicillin in a 1-L volume jar fermenter, and the culture was started at 37° C. and pH 6.7 and continued for 20 hours. After 6 hours of the culture, the glycerol concentration in the medium was controlled to be 0 to 5 g/L, 5 to 10 g/L, or 10 to 15 g/L by adding an aqueous solution of glycerol (700 g/L) dropwise.

Composition of C medium:

| | | |
|---|---|---|
| Glycerol | 30 | g/L |
| Mameno*¹⁾ | 11 | mL/L |
| KH₂PO₄ | 1.0 | g/L |
| MgSO₄•7H₂O | 1.0 | g/L |
| FeSO₄•7H₂O | 30 | mg/L |
| MnSO₄•4H₂O | 30 | mg/L |
| Betaine*²⁾ | 2 | g/L |

*¹⁾Soy bean hydrolysate (total nitrogen: 0.35 g/L)
*²⁾N,N,N-Trimethylglycine

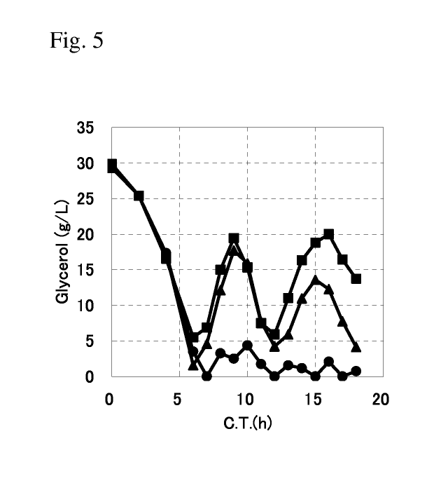

The results of the culture are shown in Table 6, and change in the glycerol concentration over time and the change in the cell growth over time are shown in FIGS. 4 and 5.

When the glycerol concentration was controlled to be 5 to 10 g/L or 10 to 15 g/L, the accumulation and productivity of L-threonine were improved as compared to when the glycerol concentration was controlled to be 0 to 5 g/L.

TABLE 6
Results of culture

| | | | | Consumed | Thr |
|---|---|---|---|---|---|
| | | Thr | Thr | glycerol | production |
| Controlled | | yield | productivity | conc. | amount |
| concentration | | (%) | (g/L/h) | (g/fermenter) | (g/L) |
| 0-5 (1.6 ± 1.4) | g/L | 32.9 | 2.23 | 37.7 | 41.2 |
| 5-10 (9.1 ± 5.3) | g/L | 34.2 | 2.28 | 39.0 | 43.4 |
| 10-15 (13.2 ± 5.5) | g/L | 34.6 | 2.31 | 40.6 | 43.9 |

*) Average glycerol concentrations after 6 hours and deviations thereof are mentioned in the parentheses in the table.

Explanation of Sequence Listing

SEQ ID NO: 1: gldA gene sequence of Escherichia coli (1104 bp)

SEQ ID NO: 2: GldA amino acid sequence of Escherichia coli (367 AA)

SEQ ID NO: 3: dakA1 gene sequence of Saccharomyces cerevisiae (1755 bp)

SEQ ID NO: 4: DakA amino acid sequence of Saccharomyces cerevisiae (584 AA)

SEQ ID NO: 5: dhbK1 gene sequence of Agrobacterium tumefaciens (1695 bp)

SEQ ID NO: 6: Dhbk1 amino acid sequence of Agrobacterium tumefaciens (564 AA)

SEQ ID NO: 7: dhaK gene sequence of Citrobacter freundii (1659 bp)

SEQ ID NO: 8: DhaK amino acid sequence of Citrobacter freundii (552 AA)

SEQ ID NO: 9: attR-cat-attL-ptacM-SD-spacer sequence (1740 bp)

SEQ ID NO: 10: tacM promoter (80 bp)

SEQ ID NO: 11: PtacMgldA::Cm sequence

SEQ ID NO: 12: atL-Ptac-gldA (PCR primer for enhancing gldA on chromosome)

SEQ ID NO: 13: atR-Ptac-fsaB1 (PCR primer for enhancing gldA on chromosome)

SEQ ID NO: 14: pMW-dak1F (primer for dakA cloning)

SEQ ID NO: 15: pMW-dak1R (primer for dakA cloning)

SEQ ID NO: 16: glpF gene sequence of Escherichia coli (846 bp)

SEQ ID NO: 17: GlpF amino acid sequence of Escherichia coli (281 AA)

SEQ ID NO: 18: tpiA gene sequence of Escherichia coli (768 bp)

SEQ ID NO: 19: TpiA amino acid sequence of Escherichia coli (255 AA)

SEQ ID NO: 20: fbaA gene sequence of Escherichia coli (1080 bp)

SEQ ID NO: 21: FbaA amino acid sequence of Escherichia coli (359 AA)

SEQ ID NO: 22: glpX gene sequence of Escherichia coli (1011 bp)

SEQ ID NO: 23: GlpX amino acid sequence of Escherichia coli (336 AA)

SEQ ID NO: 24: glpK gene sequence of Escherichia coli (1509 bp)

SEQ ID NO: 25: GlpK amino acid sequence of Escherichia coli (502 AA)

SEQ ID NO: 26: glpA gene sequence of Escherichia coli (1629 bp)

SEQ ID NO: 27: GlpA amino acid sequence of Escherichia coli (542 AA)

SEQ ID NO: 28: glpB gene sequence of Escherichia coli (1260 bp)

SEQ ID NO: 29: GlpB amino acid sequence of Escherichia coli (419 AA)

SEQ ID NO: 30: glpC gene sequence of Escherichia coli (1191 bp)

SEQ ID NO: 31: GlpC amino acid sequence of Escherichia coli (396 AA)

SEQ ID NO: 32: glpD gene sequence of Escherichia coli (1506 bp)

SEQ ID NO: 33: GlpD amino acid sequence of Escherichia coli (501 AA)

SEQ ID NO: 34: dhaK gene sequence of Escherichia coli (1071 bp)

SEQ ID NO: 35: DhaK amino acid sequence of Escherichia coli (356 AA)

SEQ ID NO: 36: dhaL gene sequence of Escherichia coli (633 bp)

SEQ ID NO: 37: DhaL amino acid sequence of Escherichia coli (210 AA)

SEQ ID NO: 38: dhaM gene sequence of Escherichia coli (1419 bp)

SEQ ID NO: 39: DhaM amino acid sequence of Escherichia coli (472 AA)

SEQ ID NO: 40: Dihydroxyacetone kinase gene of Schizosaccharomyces pombe (1776 bp)

SEQ ID NO: 41: Dihydroxyacetone kinase of Schizosaccharomyces pombe (591 AA)

SEQ ID NO: 42: Dihydroxyacetone kinase gene of Pichia angusta (1830 bp)

SEQ ID NO: 43: Dihydroxyacetone kinase of Pichia angusta (609 AA)

SEQ ID NO: 44: Dihydroxyacetone kinase gene of Pichia pastoris (1827 bp)

SEQ ID NO: 45: Dihydroxyacetone kinase of Pichia pastoris (608 AA)

SEQ ID NO: 46: Dihydroxyacetone kinase gene of Debaryomyces hansenii (1824 bp)

SEQ ID NO: 47: Dihydroxyacetone kinase of Debaryomyces hansenii (607 AA)

SEQ ID NO: 48: Dihydroxyacetone kinase gene of Escherichia blattae (1752 bp)

SEQ ID NO: 49: Dihydroxyacetone kinase of Escherichia blattae (583 AA)

SEQ ID NO: 50: Dihydroxyacetone kinase gene of Enterobacter sp. 638 (1647 bp)

SEQ ID NO: 51: Dihydroxyacetone kinase of Enterobacter sp. 638 (548 AA)

SEQ ID NO: 52: Dihydroxyacetone kinase gene of Psychromonas sp. CNPT3 (1695 bp)

SEQ ID NO: 53: Dihydroxyacetone kinase of Psychromonas sp. CNPT3 (564 AA)

SEQ ID NO: 54: Dihydroxyacetone kinase gene of Stappia aggregata (1647 bp)

SEQ ID NO: 55: Dihydroxyacetone kinase of Stappia aggregata (548 AA)

SEQ ID NO: 56: Dihydroxyacetone kinase gene of Rhizobium leguminosarum bv. viciae 3841 (1641 bp)

SEQ ID NO: 57: Dihydroxyacetone kinase of Rhizobium leguminosarum by. viciae 3841 (546 AA)

SEQ ID NO: 58: Dihydroxyacetone kinase gene of Myxococcus xanthus DK 1622 (1701 bp)

SEQ ID NO: 59: Dihydroxyacetone kinase of Myxococcus xanthus DK 1622 (566 AA)

SEQ ID NO: 60: Dihydroxyacetone kinase gene of Burkholderia sp. 383 (1701 bp)

SEQ ID NO: 61: Dihydroxyacetone kinase of Burkholderia sp. 383 (566 AA)

SEQ ID NO: 62: Dihydroxyacetone kinase gene of Burkholderia thailandensis E264 (1704 bp)

SEQ ID NO: 63: Dihydroxyacetone kinase of Burkholderia thailandensis E264 (567 AA)

SEQ ID NO: 64: Dihydroxyacetone kinase gene of Burkholderia multivorans ATCC 17616 (1851 bp)

SEQ ID NO: 65: Dihydroxyacetone kinase of Burkholderia multivorans ATCC 17616 (616 AA)

SEQ ID NO: 66: dhaR gene of Escherichia coli (1920 bp)

SEQ ID NO: 67: DhaR amino acid sequence of Escherichia coli (639 AA)

SEQ ID NO: 68: fsaA gene of Escherichia coli (663 bp)

SEQ ID NO: 69: FsaA amino acid sequence of Escherichia coli (220 AA)

SEQ ID NO: 70: fsaB gene of Escherichia coli (663 bp)

SEQ ID NO: 71: FsaB amino acid sequence of Escherichia coli (220 AA)

SEQ ID NO: 72: fbaB gene of Escherichia coli (1053 bp)

SEQ ID NO: 73 : FbaB amino acid sequence of Escherichia coli (350 AA)

SEQ ID NO: 74: gldA gene of Shigella dysenteriae Sd197 (1143 bp)

SEQ ID NO: 75: GldA amino acid sequence of Shigella dysenteriae Sd197 (380 AA)

SEQ ID NO: 76: gldA gene of Salmonella typhimurium LT2 (1104 bp)

SEQ ID NO: 77: GldA amino acid sequence of Salmonella typhimurium LT2 (367 AA)

SEQ ID NO: 78: gldA gene of Pseudomonas putida (1098 bp)

SEQ ID NO: 79: GldA amino acid sequence of Pseudomonas putida (365 AA)

SEQ ID NO: 80: gldA gene of Bacillus coagulans 36D1 (1104 bp)

SEQ ID NO: 81: GldA amino acid sequence of Bacillus coagulans 36D1 (367 AA)

SEQ ID NO: 82: fbp gene of Escherichia coli (999 bp)

SEQ ID NO: 83: Fbp amino acid sequence of Escherichia coli (322 AA)

SEQ ID NO: 84: ybhA gene of Escherichia coli (819 bp)

SEQ ID NO: 85: YbhA amino acid sequence of Escherichia coli (272 AA)

SEQ ID NO: 86: ptsl gene of Escherichia coli (1782 bp)

SEQ ID NO: 87: PtsI amino acid sequence of Escherichia coli (575 AA)

SEQ ID NO: 88: PtacM fsaB-gldA::Cm sequence

SEQ ID NO: 89: atL-Ptac-fsaB (PCR primer for enhancing fsaB+gldA on chromosome)

SEQ ID NO: 90: atR-Ptac-fsaB (PCR primer for enhancing fsaB+gldA on chromosome)

SEQ ID NO: 91: PtacM2gldA::Cm sequence

SEQ ID NO: 92: attR-cat-attL-PtacM2-SD-spacer sequence

SEQ ID NO: 93: atL-Ptac-gldA (PCR primer for enhancing gldA on chromosome)

SEQ ID NO: 94: atR-Ptac-fsaB 1 (PCR primer for enhancing gldA on chromosome)

### INDUSTRIAL APPLICABILITY

According to the present invention, in a method for producing an L-amino acid from glycerol by fermentation using a microorganism belonging to the family Enterobacteriaceae and having an L-amino acid producing ability, productivity of L-amino acid fermentation can be improved.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

1. A method for producing an L-amino acid comprising:
A) culturing a bacterium, which belongs to the family Enterobacteriaceae and is able to produce an L-amino acid, in a fermentation medium containing glycerol, and B) collecting the L-amino acid from the fermentation medium, wherein the culturing is performed as a fed-batch culture or a continuous culture, and a feed medium comprising glycerol is added to the fermentation medium so that the glycerol concentration of the fermentation medium is 5 g/L or higher.

2. The method according to claim 1, wherein the glycerol concentration is 5 g/L or higher for a period which is at least 50% or more of the entire culture period.

3. The method according to claim 1, wherein the culturing comprises a proliferation phase during which the bacterium proliferates, and a production phase during which the L-amino acid is produced, wherein the production phase occurs after the proliferation phase, and wherein the production phase is at least 70% or more of the entire culture period, and the glycerol concentration is 5 g/L or higher for a period which is at least 70% or more of the production phase.

4. The method according to claim 1, wherein an activity of an enzyme of the dihydroxyacetone pathway has been enhanced in the bacterium.

5. The method according to claim 4, wherein the enzyme is selected from the group consisting of glycerol dehydrogenase, dihydroxyacetone kinase, fructose-6-phosphate aldolase, and combinations thereof.

6. The method according to claim 5, wherein the enzyme comprises a mixture of glycerol dehydrogenase, dihydroxyacetone kinase, and fructose-6-phosphate aldolase.

7. The method according to claim 4, wherein, the activity of the enzyme has been enhanced by increasing copy number of a gene coding for the enzyme, or by modifying an expression control sequence of the gene.

8. The method according to claim 1, wherein the bacterium is an Escherichia bacterium.

9. The method according to claim 8, wherein the bacterium is Escherichia coli.

10. The method according to claim 1, wherein the L-amino acid is L-lysine or L-threonine.


SEQUENCE LISTING (94 sequences)

SEQ ID NO: 1 - DNA, 1104 bp, Escherichia coli
  CDS (1)..(1104)
atggaccgcattattcaatcaccgggtaaatacatccagggcgctgatgtgattaatcgtctgggcgaatacctgaagccgctggcagaacgctggttagtggtgggtgacaaatttgttttaggttttgctcaatccactgtcgagaaaagctttaaagatgctggactggtagtagaaattgcgccgtttggcggtgaatgttcgcaaaatgagatcgaccgtctgcgtggcatcgcggagactgcgcagtgtggcgcaattctcggtatcggtggcggaaaaaccctcgatactgccaaagcactggcacatttcatgggtgttccggtagcgatcgcaccgactatcgcctctaccgatgcaccgtgcagcgcattgtctgttatctacaccgatgagggtgagtttgaccgctatctgctgttgccaaataacccgaatatggtcattgtcgacaccaaaatcgtcgctggcgcacctgcacgtctgttagcggcgggtatcggcgatgcgctggcaacctggtttgaagcgcgtgcctgctctcgtagcggcgcgaccaccatggcgggcggcaagtgcacccaggctgcgctggcactggctgaactgtgctacaacaccctgctggaagaaggcgaaaaagcgatgcttgctgccgaacagcatgtagtgactccggcgctggagcgcgtgattgaagcgaacacctatttgagcggtgttggttttgaaagtggtggtctggctgcggcgcacgcagtgcataacggcctgaccgctatcccggacgcgcatcactattatcacggtgaaaaagtggcattcggtacgctgacgcagctggttctggaaaatgcgccggtggaggaaatcgaaaccgtagctgcccttagccatgcggtaggtttgccaataactctcgctcaactggatattaaagaagatgtcccggcgaaaatgcgaattgtggcagaagcggcatgtgcagaaggtgaaaccattcacaacatgcctggcggcgcgacgccagatcaggtttacgccgctctgctggtagccgaccagtacggtcagcgtttcctgcaagagtgggaataa

SEQ ID NO: 2 - protein, 367 aa, Escherichia coli
MDRIIQSPGKYIQGADVINRLGEYLKPLAERWLVVGDKFVLGFAQSTVEKSFKDAGLVVEIAPFGGECSQNEIDRLRGIAETAQCGAILGIGGGKTLDTAKALAHFMGVPVAIAPTIASTDAPCSALSVIYTDEGEFDRYLLLPNNPNMVIVDTKIVAGAPARLLAAGIGDALATWFEARACSRSGATTMAGGKCTQAALALAELCYNTLLEEGEKAMLAAEQHVVTPALERVIEANTYLSGVGFESGGLAAAHAVHNGLTAIPDAHHYYHGEKVAFGTLTQLVLENAPVEEIETVAALSHAVGLPITLAQLDIKEDVPAKMRIVAEAACAEGETIHNMPGGATPDQVYAALLVADQYGQRFLQEWE

SEQ ID NO: 3 - DNA, 1755 bp, Saccharomyces cerevisiae
  CDS (1)..(1755)
atgtccgctaaatcgtttgaagtcacagatccagtcaattcaagtctcaaagggtttgcccttgctaacccctccattacgctggtccctgaagaaaaaattctcttcagaaagaccgattccgacaagatcgcattaatttctggtggtggtagtggacatgaacctacacacgccggtttcattggtaagggtatgttgagtggcgccgtggttggcgaaatttttgcatccccttcaacaaaacagattttaaatgcaatccgtttagtcaatgaaaatgcgtctggcgttttattgattgtgaagaactacacaggtgatgttttgcattttggtctgtccgctgagagagcaagagccttgggtattaactgccgcgttgctgtcataggtgatgatgttgcagttggcagagaaaagggtggtatggttggtagaagagcattggcaggtaccgttttggttcataagattgtaggtgccttcgcagaagaatattctagtaagtatggcttagacggtacagctaaagtggctaaaattatcaacgacaatttggtgaccattggatcttctttagaccattgtaaagttcctggcaggaaattcgaaagtgaattaaacgaaaaacaaatggaattgggtatgggtattcataacgaacctggtgtgaaagttttagaccctattccttctaccgaagacttgatctccaagtatatgctaccaaaactattggatccaaacgataaggatagagcttttgtaaagtttgatgaagatgatgaagttgtcttgttagttaacaatctcggcggtgtttctaattttgttattagttctatcacttccaaaactacggatttcttaaaggaaaattacaacataaccccggttcaaacaattgctggcacattgatgacctccttcaatggtaatgggttcagtatcacattactaaacgccactaaggctacaaaggctttgcaatctgattttgaggagatcaaatcagtactagacttgttgaacgcatttacgaacgcaccgggctggccaattgcagattttgaaaagacttctgccccatctgttaacgatgacttgttacataatgaagtaacagcaaaggccgtcggtacctatgactttgacaagtttgctgagtggatgaagagtggtgctgaacaagttatcaagagcgaaccgcacattacggaactagacaatcaagttggtgatggtgattgtggttacactttagtggcaggagttaaaggcatcaccgaaaaccttgacaagctgtcgaaggactcattatctcaggcggttgcccaaatttcagatttcattgaaggctcaatgggaggtacttctggtggtttatattctattcttttgtcgggtttttcacacggattaattcaggtttgtaaatcaaaggatgaacccgtcactaaggaaattgtggctaagtcactcggaattgcattggatactttatacaaatatacaaaggcaaggaagggatcatccaccatgattgatgctttagaaccattcgttaaagaatttactgcatctaaggatttcaataaggcggtaaaagctgcagaggaaggtgctaaatccactgctacattcgaggccaaatttggcagagcttcgtatgtcggcgattcatctcaagtagaagatcctggtgcagtaggcctatgtgagtttttgaagggggttcaaagcgccttgtaa

SEQ ID NO: 4 - protein, 584 aa, Saccharomyces cerevisiae
MSAKSFEVTDPVNSSLKGFALANPSITLVPEEKILFRKTDSDKIALISGGGSGHEPTHAGFIGKGMLSGAVVGEIFASPSTKQILNAIRLVNENASGVLLIVKNYTGDVLHFGLSAERARALGINCRVAVIGDDVAVGREKGGMVGRRALAGTVLVHKIVGAFAEEYSSKYGLDGTAKVAKIINDNLVTIGSSLDHCKVPGRKFESELNEKQMELGMGIHNEPGVKVLDPIPSTEDLISKYMLPKLLDPNDKDRAFVKFDEDDEVVLLVNNLGGVSNFVISSITSKTTDFLKENYNITPVQTIAGTLMTSFNGNGFSITLLNATKATKALQSDFEEIKSVLDLLNAFTNAPGWPIADFEKTSAPSVNDDLLHNEVTAKAVGTYDFDKFAEWMKSGAEQVIKSEPHITELDNQVGDGDCGYTLVAGVKGITENLDKLSKDSLSQAVAQISDFIEGSMGGTSGGLYSILLSGFSHGLIQVCKSKDEPVTKEIVAKSLGIALDTLYKYTKARKGSSTMIDALEPFVKEFTASKDFNKAVKAAEEGAKSTATFEAKFGRASYVGDSSQVEDPGAVGLCEFLKGVQSAL

SEQ ID NO: 5 - DNA, 1695 bp, Agrobacterium tumefaciens
  CDS (1)..(1695)
atgaagaagctcatcaacgatccatccaccgttgtccgggacatgctggagggcatcgtggcgctcagccccgaaaccattctgcttcaggatgagaacgtggtcataaggtccggcctgcctgaagcggaaaagcgcaaggttgcagtgctttcgggcggtggcagcggacatgagccggcccacgccggttatgtcggcacgggcatgttgacggttgcggtggcgggcgatgtcttcacttcgccgagcaccgacgcggttctcgccggcatcagggccgcggccggccctgccggtgcgctggtcatcgtcaagaactataccggcgaccggctgaatttcggcttggcggcggagctggcgagggccgaaggaatccctgtcgagatcgttgtcgtcgccgacgacgttgccttgaaggatacggttcccgccgagcgccgccgcgggattgcggggacggtgctcgtgcacaagctcgcgggggccgcagcggaaaagggccttcctcttcaagaggtggcccgcatcgcccgggacgctgccgccaagctatcttccatgggtgtctcgctgggatcctgcacgctgccggctgtcggcaagccgggcttcgtgcttggcgagaccgaaatcgaagtcgggctcggtatccatggcgagcagggcgtgcagcggatgcccattgcctcggctgacgcgctcgtgcagctggtgatcgaaacgatcgaagccgacggcaagctcgccggcggcaatcgcgtcgctctgctggtcaacggcctgggggcaacgccgccgatggaactcgccatcgtcgcacggtcggcagtcgcgcggctggaggcgaaaggcatcgtcgtggaacgtgcctgggccggcaccttcctttcagccctcgatatgcccgggttttcgttgtcggtcatgcaggttgacgacgcagcgctcagcctcatcgacgcgccaaccgaggctggcgcatggccgcgcggcggtgcggtgaaccgcaagcgggttctgccttcggcaaacgccgaaaagaccgtggttgcgacaaacaagatgacggcggccggcgagcggcttcgttcgggcgcggaacggtccgcgagagccctgatcgctgcggagcccaggctgacgcaactcgatagcgttgcaggtgacggcgacctcggcgccagcatggtgcgtggcggcgaggcgatccttgcgctgccgaaagagagtttcggcgacgtctccgatggattgatggcgatggccaatgcgatgcgcaaggccatcggcggaagctcggggccgttctatgcgacaggcctcatgcgcgcttcgcgacagctggcagggatcgatgagccagcggcccagcagatggcggaagcattcgtggcggctgttgcggcggtctcggaacttggcggtgcgaaaccgggcgatcgcacgatgatcgatgcgctttatccggcagcgaaaaccttcagggacaagcttgtgacaggcgcttcggcagaagaagcctggcaatccgcggtggcggcgggcgaggtgggcgcagaggcgacagcgtccatgaagccacggctcgggcgcgcaagttacctcggtgagcgggccgtcggccatcccgatgggggcgcggtcgccgttggcatctggctcaaagctatcgaggctgcgatctcgtga

SEQ ID NO: 6 - protein, 564 aa, Agrobacterium tumefaciens
MKKLINDPSTVVRDMLEGIVALSPETILLQDENVVIRSGLPEAEKRKVAVLSGGGSGHEPAHAGYVGTGMLTVAVAGDVFTSPSTDAVLAGIRAAAGPAGALVIVKNYTGDRLNFGLAAELARAEGIPVEIVVVADDVALKDTVPAERRRGIAGTVLVHKLAGAAAEKGLPLQEVARIARDAAAKLSSMGVSLGSCTLPAVGKPGFVLGETEIEVGLGIHGEQGVQRMPIASADALVQLVIETIEADGKLAGGNRVALLVNGLGATPPMELAIVARSAVARLEAKGIVVERAWAGTFLSALDMPGFSLSVMQVDDAALSLIDAPTEAGAWPRGGAVNRKRVLPSANAEKTVVATNKMTAAGERLRSGAERSARALIAAEPRLTQLDSVAGDGDLGASMVRGGEAILALPKESFGDVSDGLMAMANAMRKAIGGSSGPFYATGLMRASRQLAGIDEPAAQQMAEAFVAAVAAVSELGGAKPGDRTMIDALYPAAKTFRDKLVTGASAEEAWQSAVAAGEVGAEATASMKPRLGRASYLGERAVGHPDGGAVAVGIWLKAIEAAIS

SEQ ID NO: 7 - DNA, 1659 bp, Citrobacter freundii
  CDS (1)..(1659)
atgtctcaattcttttttaaccaacgcacccatcttgtgagcgacgtcatcgacggggcgattatcgccagcccatggaataacctggcgcgtctggaaagcgatccggccattcgcatcgtggtccgtcgtgaccttaataaaaataacgtagcggtcatttccggcggcggttcgggacacgaacccgcgcacgttgggtttatcggtaaaggcatgctaaccgctgcggtctgcggcgacgttttcgcctccccgagcgtggatgctgtactgaccgcgattcaggcggtgaccggtgaggctggctgtttgttgattgtgaaaaactacaccggtgaccgtcttaatttcggtctcgccgccgagaaggcgcgtcgccttggctataacgttgaaatgctgattgtcggcgacgacatctccctgccggataacaaacacccacgtggcattgcgggaactatcctggtgcataaaatcgcaggctattttgccgaacgcggctataacctcgccaccgtcctgcgtgaagcgcagtacgcagccagcaacacctttagcctgggcgtagcgctttccagctgtcatctgccgcaagaaaccgacgcagcccctcgtcatcatccgggtcatgcggagctgggtatgggaattcacggcgaaccaggcgcatcggttatcgacacccaaaacagtgcgcaagtggtaaacctgatggtggataaactgctggccgccctgcctgaaaccggtcgtctggcggtgatgattaataatcttggcggcgtttccgtggccgaaatggccatcatcacccgcgaactcgccagcagcccgctgcactcgcgtatcgactggctaattggcccggcctcgctggtcaccgcgctggatatgaaaggcttctcactgacggccatcgtgctggaagagagcatcgaaaaagcactgctcaccgaagtggaaaccagcaactggccgacgccggtcccaccgcgtgaaatcacctgcgtagtgtcatctcacgctagcgcccgcgtggaattccagccttcggcaaacgccctggtggccgggattgtggagctggtcaccgcaaccctttccgatctggagactcatctgaatgcgctggacgccaaagtcggcgatggcgataccggttcgacctttgccgccgcggcgcgtgaaattgccagcctgctgcatcgccagcagctgccgctgaataaccttgccacgctgttcgcgctgattggcgaacgtctgaccgtggtgatgggcggttccagcggtgtgctgatgtcaatcttctttaccgccgccgggcagaaactggaacagggcgctaacgttgtcgaagcgctaaatacggggctggcgcagatgaagttctacggcggcgcagacgaaggcgatcgcacgatgattgatgcgctgcaaccggccctgacctcgctgctcgcacagccgaaaaatctgcaggccgcattcgacgccgcgcaagcgggagccgaacgaacctgtttgtcgagcaaagccaatgcgggtcgcgcatcgtatctgagcagcgaaagcctgctcggaaatatggaccccggcgcgcagcgcctagcgatggtgtttaaagcgctagcggagagtgagctgggctaa

SEQ ID NO: 8 - protein, 552 aa, Citrobacter freundii
MSQFFFNQRTHLVSDVIDGAIIASPWNNLARLESDPAIRIVVRRDLNKNNVAVISGGGSGHEPAHVGFIGKGMLTAAVCGDVFASPSVDAVLTAIQAVTGEAGCLLIVKNYTGDRLNFGLAAEKARRLGYNVEMLIVGDDISLPDNKHPRGIAGTILVHKIAGYFAERGYNLATVLREAQYAASNTFSLGVALSSCHLPQETDAAPRHHPGHAELGMGIHGEPGASVIDTQNSAQVVNLMVDKLLAALPETGRLAVMINNLGGVSVAEMAIITRELASSPLHSRIDWLIGPASLVTALDMKGFSLTAIVLEESIEKALLTEVETSNWPTPVPPREITCVVSSHASARVEFQPSANALVAGIVELVTATLSDLETHLNALDAKVGDGDTGSTFAAAAREIASLLHRQQLPLNNLATLFALIGERLTVVMGGSSGVLMSIFFTAAGQKLEQGANVVEALNTGLAQMKFYGGADEGDRTMIDALQPALTSLLAQPKNLQAAFDAAQAGAERTCLSSKANAGRASYLSSESLLGNMDPGAQRLAMVFKALAESELG

SEQ ID NO: 9 - DNA, 1740 bp, Artificial
  attR-cat-attL-PtacM-SD-spacer
tctagacgctcaagttagtataaaaaagctgaacgagaaacgtaaaatgatataaatatcaatatattaaattagattttgcataaaaaacagactacataatactgtaaaacacaacatatgcagtcactatgaatcaactacttagatggtattagtgacctgtaacagactgcagtggtcgaaaaaaaaagcccgcactgtcaggtgcgggcttttttctgtgttaagcttcgacgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcggatccggccaagctagcttggctctagctagagcgcccggttgacgctgctagtgttacctagcgatttgtatcttactgcatgttacttcatgttgtcaatacctgtttttcgtgcgacttatcaggctgtctacttatccggagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcgaaatcaaataatgattttattttgactgatagtgacctgttcgttgcaacaaattgataagcaatgcttttttataatgccaacttagtataaaaaagcaggcttcaagatctctccccatccccctgttcacaattaatcatcggctcgtataatgtgtggaattgtgagcggataacaatttcacacaggagactgcc

SEQ ID NO: 10 - DNA, 80 bp, Artificial
  PtacM
ctccccatccccctgttcacaattaatcatcggctcgtataatgtgtggaattgtgagcggataacaatttcacacagga

SEQ ID NO: 11 - DNA, 2844 bp, Artificial
  PtacMgldA::Cm
tctagacgctcaagttagtataaaaaagctgaacgagaaacgtaaaatgatataaatatcaatatattaaattagattttgcataaaaaacagactacataatactgtaaaacacaacatatgcagtcactatgaatcaactacttagatggtattagtgacctgtaacagactgcagtggtcgaaaaaaaaagcccgcactgtcaggtgcgggcttttttctgtgttaagcttcgacgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcggatccggccaagctagcttggctctagctagagcgcccggttgacgctgctagtgttacctagcgatttgtatcttactgcatgttacttcatgttgtcaatacctgtttttcgtgcgacttatcaggctgtctacttatccggagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcgaaatcaaataatgattttattttgactgatagtgacctgttcgttgcaacaaattgataagcaatgcttttttataatgccaacttagtataaaaaagcaggcttcaagatctctccccatccccctgttcacaattaatcatcggctcgtataatgtgtggaattgtgagcggataacaatttcacacaggagactgccatggaccgcattattcaatcaccgggtaaatacatccagggcgctgatgtgattaatcgtctgggcgaatacctgaagccgctggcagaacgctggttagtggtgggtgacaaatttgttttaggttttgctcaatccactgtcgagaaaagctttaaagatgctggactggtagtagaaattgcgccgtttggcggtgaatgttcgcaaaatgagatcgaccgtctgcgtggcatcgcggagactgcgcagtgtggcgcaattctcggtatcggtggcggaaaaaccctcgatactgccaaagcactggcacatttcatgggtgttccggtagcgatcgcaccgactatcgcctctaccgatgcaccgtgcagcgcattgtctgttatctacaccgatgagggtgagtttgaccgctatctgctgttgccaaataacccgaatatggtcattgtcgacaccaaaatcgtcgctggcgcacctgcacgtctgttagcggcgggtatcggcgatgcgctggcaacctggtttgaagcgcgtgcctgctctcgtagcggcgcgaccaccatggcgggcggcaagtgcacccaggctgcgctggcactggctgaactgtgctacaacaccctgctggaagaaggcgaaaaagcgatgcttgctgccgaacagcatgtagtgactccggcgctggagcgcgtgattgaagcgaacacctatttgagcggtgttggttttgaaagtggtggtctggctgcggcgcacgcagtgcataacggcctgaccgctatcccggacgcgcatcactattatcacggtgaaaaagtggcattcggtacgctgacgcagctggttctggaaaatgcgccggtggaggaaatcgaaaccgtagctgcccttagccatgcggtaggtttgccaataactctcgctcaactggatattaaagaagatgtcccggcgaaaatgcgaattgtggcagaagcggcatgtgcagaaggtgaaaccattcacaacatgcctggcggcgcgacgccagatcaggtttacgccgctctgctggtagccgaccagtacggtcagcgtttcctgcaagagtgggaataa

SEQ ID NO: 12 - DNA, 77 bp, Artificial
  atL-ptac-gldA
acatcagcgccctggatgtatttacccggtgattgaataatgcggtccatggcagtctcctgtgtgaaattgttatc

SEQ ID NO: 13 - DNA, 77 bp, Artificial
  atL-Ptac-fsaB1
aacgccgcctctgccgacgctatcgccagcctgctgcaacatgaactggaactgtaaatctagacgctcaagttagt

SEQ ID NO: 14 - DNA, 50 bp, Artificial
  pMW-dak1F
tgattacgccaagcttaggaggttaaatgtccgctaaatcgtttgaagtc

SEQ ID NO: 15 - DNA, 52 bp, Artificial
  pMW-dak1R
atcctctagagtcgacgcggccgctacttacaaggcgctttgaacccccttc

SEQ ID NO: 16 - DNA, 846 bp, Escherichia coli
  CDS (1)..(846)
atgagtcaaacatcaaccttgaaaggccagtgcattgctgaattcctcggtaccgggttgttgattttcttcggtgtgggttgcgttgcagcactaaaagtcgctggtgcgtcttttggtcagtgggaaatcagtgtcatttggggactgggggtggcaatggccatctacctgaccgcaggggtttccggcgcgcatcttaatcccgctgttaccattgcattgtggctgtttgcctgtttcgacaagcgcaaagttattccttttatcgtttcacaagttgccggcgctttctgtgctgcggctttagtttacgggctttactacaatttatttttcgacttcgagcagactcatcacattgttcgcggcagcgttgaaagtgttgatctggctggcactttctctacttaccctaatcctcatatcaattttgtgcaggctttcgcagttgagatggtgattaccgctattctgatggggctgatcctggcgttaacggacgatggcaacggtgtaccacgcggccctttggctcccttgctgattggtctactgattgcggtcattggcgcatctatgggcccattgacaggttttgccatgaacccagcgcgtgacttcggtccgaaagtctttgcctggctggcgggctggggcaatgtcgcctttaccggcggcagagacattccttacttcctggtgccgcttttcggccctatcgttggcgcgattgtaggtgcatttgcctaccgcaaactgattggtcgccatttgccttgcgatatctgtgttgtggaagaaaaggaaaccacaactccttcagaacaaaaagcttcgctgtaa

SEQ ID NO: 17 - protein, 281 aa, Escherichia coli
MSQTSTLKGQCIAEFLGTGLLIFFGVGCVAALKVAGASFGQWEISVIWGLGVAMAIYLTAGVSGAHLNPAVTIALWLFACFDKRKVIPFIVSQVAGAFCAAALVYGLYYNLFFDFEQTHHIVRGSVESVDLAGTFSTYPNPHINFVQAFAVEMVITAILMGLILALTDDGNGVPRGPLAPLLIGLLIAVIGASMGPLTGFAMNPARDFGPKVFAWLAGWGNVAFTGGRDIPYFLVPLFGPIVGAIVGAFAYRKLIGRHLPCDICVVEEKETTTPSEQKASL

SEQ ID NO: 18 - DNA, 768 bp, Escherichia coli
  CDS (1)..(768)
atgcgacatcctttagtgatgggtaactggaaactgaacggcagccgccacatggttcacgagctggtttctaacctgcgtaaagagctggcaggtgttgctggctgtgcggttgcaatcgcaccaccggaaatgtatatcgatatggcgaagcgcgaagctgaaggcagccacatcatgctgggtgcgcaaaacgtggacctgaacctgtccggcgcattcaccggtgaaacctctgctgctatgctgaaagacatcggcgcacagtacatcatcatcggtcactctgaacgtcgtacttaccacaaagaatctgacgaactgatcgcgaaaaaattcgcggtgctgaaagagcagggcctgactccggttctgtgcatcggtgaaaccgaagctgaaaatgaagcgggcaaaactgaagaagtttgcgcacgtcagatcgacgcggtactgaaaactcagggtgctgcggcattcgaaggtgcggttatcgcttacgaacctgtatgggcaatcggtactggcaaatctgcaactccggctcaggcacaggctgttcacaaattcatccgtgaccacatcgctaaagttgacgctaacatcgctgaacaagtgatcattcagtacggcggctctgtaaacgcgtctaacgctgcagaactgtttgctcagccggatatcgacggcgcgctggttggtggtgcttctctgaaagctgacgccttcgcagtaatcgttaaagctgcagaagcggctaaacaggcttaa

SEQ ID NO: 19 - protein, 255 aa, Escherichia coli
MRHPLVMGNWKLNGSRHMVHELVSNLRKELAGVAGCAVAIAPPEMYIDMAKREAEGSHIMLGAQNVDLNLSGAFTGETSAAMLKDIGAQYIIIGHSERRTYHKESDELIAKKFAVLKEQGLTPVLCIGETEAENEAGKTEEVCARQIDAVLKTQGAAAFEGAVIAYEPVWAIGTGKSATPAQAQAVHKFIRDHIAKVDANIAEQVIIQYGGSVNASNAAELFAQPDIDGALVGGASLKADAFAVIVKAAEAAKQA

SEQ ID NO: 20 - DNA, 1080 bp, Escherichia coli
  CDS (1)..(1080)
atgtctaagatttttgatttcgtaaaacctggcgtaatcactggtgatgacgtacagaaagttttccaggtagcaaaagaaaacaacttcgcactgccagcagtaaactgcgtcggtactgactccatcaacgccgtactggaaaccgctgctaaagttaaagcgccggttatcgttcagttctccaacggtggtgcttcctttatcgctggtaaaggcgtgaaatctgacgttccgcagggtgctgctatcctgggcgcgatctctggtgcgcatcacgttcaccagatggctgaacattatggtgttccggttatcctgcacactgaccactgcgcgaagaaactgctgccgtggatcgacggtctgttggacgcgggtgaaaaacacttcgcagctaccggtaagccgctgttctcttctcacatgatcgacctgtctgaagaatctctgcaagagaacatcgaaatctgctctaaatacctggagcgcatgtccaaaatcggcatgactctggaaatcgaactgggttgcaccggtggtgaagaagacggcgtggacaacagccacatggacgcttctgcactgtacacccagccggaagacgttgattacgcatacaccgaactgagcaaaatcagcccgcgtttcaccatcgcagcgtccttcggtaacgtacacggtgtttacaagccgggtaacgtggttctgactccgaccatcctgcgtgattctcaggaatatgtttccaagaaacacaacctgccgcacaacagcctgaacttcgtattccacggtggttccggttctactgctcaggaaatcaaagactccgtaagctacggcgtagtaaaaatgaacatcgataccgatacccaatgggcaacctgggaaggcgttctgaactactacaaagcgaacgaagcttatctgcagggtcagctgggtaacccgaaaggcgaagatcagccgaacaagaaatactacgatccgcgcgtatggctgcgtgccggtcagacttcgatgatcgctcgtctggagaaagcattccaggaactgaacgcgatcgacgttctgtaa

SEQ ID NO: 21 - protein, 359 aa, Escherichia coli
MSKIFDFVKPGVITGDDVQKVFQVAKENNFALPAVNCVGTDSINAVLETAAKVKAPVIVQFSNGGASFIAGKGVKSDVPQGAAILGAISGAHHVHQMAEHYGVPVILHTDHCAKKLLPWIDGLLDAGEKHFAATGKPLFSSHMIDLSEESLQENIEICSKYLERMSKIGMTLEIELGCTGGEEDGVDNSHMDASALYTQPEDVDYAYTELSKISPRFTIAASFGNVHGVYKPGNVVLTPTILRDSQEYVSKKHNLPHNSLNFVFHGGSGSTAQEIKDSVSYGVVKMNIDTDTQWATWEGVLNYYKANEAYLQGQLGNPKGEDQPNKKYYDPRVWLRAGQTSMIARLEKAFQELNAIDVL

SEQ ID NO: 22 - DNA, 1011 bp, Escherichia coli
  CDS (1)..(1011)
atgagacgagaacttgccatcgaattttcccgcgtcaccgaatcagcggcgctggctggctacaaatggttaggacgcggcgataaaaacaccgcggacggcgcggcggtaaacgccatgcgtattatgctcaaccaggtcaacattgacggcaccatcgtcattggtgaaggtgaaatcgacgaagcaccgatgctctacattggtgaaaaagtcggtactggtcgcggcgacgcggtagatattgctgttgatccgattgaaggcacgcgcatgacggcgatgggccaggctaacgcgctggcggtgctggcagtaggcgataaaggctgcttcctcaatgcgccggatatgtatatggagaagctgattgtcgggccgggagccaaaggcaccattgatctgaacctgccgctggcggataacctgcgcaatgtagcggcggcgctcggcaaaccgttgagcgaactgacggtaacgattctggctaaaccacgccacgatgccgttatcgctgaaatgcagcaactcggcgtacgcgtatttgctattccggacggcgacgttgcggcctcaattctcacctgtatgccagacagcgaagttgacgtgctgtacggtattggtggcgcgccggaaggcgtagtttctgcggcggtgatccgcgcattagatggcgacatgaacggtcgtctgctggcgcgtcatgacgtcaaaggcgacaacgaagagaatcgtcgcattggcgagcaggagctggcacgctgcaaagcgatgggcatcgaagccggtaaagtattgcgcctgggcgatatggcgcgcagcgataacgtcatcttctctgccaccggtattaccaaaggcgatctgctggaaggcattagccgcaaaggcaatatcgcgactaccgaaacgctgctgatccgcggcaagtcacgcaccattcgccgcattcagtccatccactatctggatcgcaaagacccggaaatgcaggtgcacatcctctga

SEQ ID NO: 23 - protein, 336 aa, Escherichia coli
MRRELAIEFSRVTESAALAGYKWLGRGDKNTADGAAVNAMRIMLNQVNIDGTIVIGEGEIDEAPMLYIGEKVGTGRGDAVDIAVDPIEGTRMTAMGQANALAVLAVGDKGCFLNAPDMYMEKLIVGPGAKGTIDLNLPLADNLRNVAAALGKPLSELTVTILAKPRHDAVIAEMQQLGVRVFAIPDGDVAASILTCMPDSEVDVLYGIGGAPEGVVSAAVIRALDGDMNGRLLARHDVKGDNEENRRIGEQELARCKAMGIEAGKVLRLGDMARSDNVIFSATGITKGDLLEGISRKGNIATTETLLIRGKSRTIRRIQSIHYLDRKDPEMQVHIL

SEQ ID NO: 24 - DNA, 1509 bp, Escherichia coli
  CDS (1)..(1509)
atgactgaaaaaaaatatatcgttgcgctcgaccagggcaccaccagctcccgcgcggtcgtaatggatcacgatgccaatatcattagcgtgtcgcagcgcgaatttgagcaaatctacccaaaaccaggttgggtagaacacgacccaatggaaatctgggccacccaaagctccacgctggtagaagtgctggcgaaagccgatatcagttccgatcaaattgcagctatcggtattacgaaccagcgtgaaaccactattgtctgggaaaaagaaaccggcaagcctatctataacgccattgtctggcagtgccgtcgtaccgcagaaatctgcgagcatttaaaacgtgacggtttagaagattatatccgcagcaataccggtctggtgattgacccgtacttttctggcaccaaagtgaagtggatcctcgaccatgtggaaggctctcgcgagcgtgcacgtcgtggtgaattgctgtttggtacggttgatacgtggcttatctggaaaatgactcagggccgtgtccatgtgaccgattacaccaacgcctctcgtaccatgttgttcaacatccataccctggactgggacgacaaaatgctggaagtgctggatattccgcgcgagatgctgccagaagtgcgtcgttcttccgaagtatacggtcagactaacattggcggcaaaggcggcacgcgtattccaatctccgggatcgccggtgaccagcaggccgcgctgtttggtcagttgtgcgtgaaagaagggatggcgaagaacacctatggcactggctgctttatgctgatgaacactggcgagaaagcggtgaaatcagaaaacggcctgctgaccaccatcgcctgcggcccgactggcgaagtgaactatgcgttggaaggtgcggtgtttatggcaggcgcatccattcagtggctgcgcgatgaaatgaagttgattaacgacgcctacgattccgaatatttcgccaccaaagtgcaaaacaccaatggtgtgtatgtggttccggcatttaccgggctgggtgcgccgtactgggacccgtatgcgcgcggggcgattttcggtctgactcgtggggtgaacgctaaccacattatacgcgcgacgctggagtctattgcttatcagacgcgtgacgtgctggaagcgatgcaggccgactctggtatccgtctgcacgccctgcgcgtggatggtggcgcagtagcaaacaatttcctgatgcagttccagtccgatattctcggcacccgcgttgagcgcccggaagtgcgcgaagtcaccgcattgggtgcggcctatctcgcaggcctggcggttggcttctggcagaacctcgacgagctgcaagagaaagcggtgattgagcgcgagttccgtccaggcatcgaaaccactgagcgtaattaccgttacgcaggctggaaaaaagcggttaaacgcgcgatggcgtgggaagaacacgacgaataa

SEQ ID NO: 25 - protein, 502 aa, Escherichia coli
MTEKKYIVALDQGTTSSRAVVMDHDANIISVSQREFEQIYPKPGWVEHDPMEIWATQSSTLVEVLAKADISSDQIAAIGITNQRETTIVWEKETGKPIYNAIVWQCRRTAEICEHLKRDGLEDYIRSNTGLVIDPYFSGTKVKWILDHVEGSRERARRGELLFGTVDTWLIWKMTQGRVHVTDYTNASRTMLFNIHTLDWDDKMLEVLDIPREMLPEVRRSSEVYGQTNIGGKGGTRIPISGIAGDQQAALFGQLCVKEGMAKNTYGTGCFMLMNTGEKAVKSENGLLTTIACGPTGEVNYALEGAVFMAGASIQWLRDEMKLINDAYDSEYFATKVQNTNGVYVVPAFTGLGAPYWDPYARGAIFGLTRGVNANHIIRATLESIAYQTRDVLEAMQADSGIRLHALRVDGGAVANNFLMQFQSDILGTRVERPEVREVTALGAAYLAGLAVGFWQNLDELQEKAVIEREFRPGIETTERNYRYAGWKKAVKRAMAWEEHDE

SEQ ID NO: 26 - DNA, 1629 bp, Escherichia coli
  CDS (1)..(1629)
atgaaaactcgcgactcgcaatcaagtgacgtgattatcattggcggcggcgcaacgggagccgggattgcccgcgactgtgccctgcgcgggctgcgcgtgattttggttgagcgccacgacatcgcaaccggtgccaccgggcgtaaccacggcctgctgcacagcggtgcgcgctatgcggtaaccgatgcggaatcggcccgcgaatgcattagtgaaaaccagatcctgaaacgcattgcacgtcactgcgttgaaccaaccaacggcctgtttatcaccctgccggaagatgacctctccttccaggccacttttattcgcgcctgcgaagaagcagggatcagcgcagaagctatagacccgcagcaagcgcgcattatcgaacctgccgttaacccggcactgattggcgcggtgaaagttccggatggcaccgttgatccatttcgtctgaccgcagcaaacatgctggatgccaaagaacacggtgccgttatccttaccgctcatgaagtcacggggctgattcgtgaaggcgcgacggtgtgcggtgttcgtgtacgtaaccatctcaccggcgaaactcaggcccttcatgcacctgtcgtggttaatgccgctgggatctgggggcaacacattgccgaatatgccgatctgcgcattcgcatgttcccggcgaaaggatcgctgctgatcatggatcaccgcattaaccagcatgtgatcaaccgctgccgtaaaccttccgacgccgatattctggtgcctggcgataccatttcgctgattggtaccacctctttacgtattgattacaacgagattgacgataatcgagtgacggcagaagaggttgatattctgctgcgtgaaggggaaaaactggcccccgtgatggcgaaaacgcgcattttgcgggcctattctggcgtgcgcccgctggttgccagcgatgacgacccgagcggacgtaacgtcagccgtggcatcgtgctgctcgaccatgctgaacgcgatggtctggacggatttatcaccatcaccggtggcaaactgatgacctatcggctgatggctgaatgggctaccgacgcggtatgccgcaaactgggcaacacgcgcccctgtacgactgccgatctggcactgcctggttcacaagaacccgctgaagttaccttgcgtaaagtcatctccctgcctgccccgctgcgcggttctgcggtttatcgtcatggcgatcgcacgcctgcctggctgagcgaaggccgtctgcaccgtagcctggtatgtgagtgcgaagcggtaactgcgggtgaagtgcagtacgcggtagaaaatttaaacgttaatagcctgctggatttacgccgtcgtacccgtgtggggatgggcacctgccagggcgaactctgcgcctgccgcgctgccggactgctgcaacgttttaacgtcacgacgtccgcgcaatctatcgagcaactttccaccttccttaacgaacgctggaaaggcgtgcaacccatcgcctggggagatgcactgcgcgaaagcgaatttacccgctgggtttatcagggattgtgtggtctggagaaggagcagaaagatgcgctttga

SEQ ID NO: 27 - protein, 542 aa, Escherichia coli
MKTRDSQSSDVIIIGGGATGAGIARDCALRGLRVILVERHDIATGATGRNHGLLHSGARYAVTDAESARECISENQILKRIARHCVEPTNGLFITLPEDDLSFQATFIRACEEAGISAEAIDPQQARIIEPAVNPALIGAVKVPDGTVDPFRLTAANMLDAKEHGAVILTAHEVTGLIREGATVCGVRVRNHLTGETQALHAPVVVNAAGIWGQHIAEYADLRIRMFPAKGSLLIMDHRINQHVINRCRKPSDADILVPGDTISLIGTTSLRIDYNEIDDNRVTAEEVDILLREGEKLAPVMAKTRILRAYSGVRPLVASDDDPSGRNVSRGIVLLDHAERDGLDGFITITGGKLMTYRLMAEWATDAVCRKLGNTRPCTTADLALPGSQEPAEVTLRKVISLPAPLRGSAVYRHGDRTPAWLSEGRLHRSLVCECEAVTAGEVQYAVENLNVNSLLDLRRRTRVGMGTCQGELCACRAAGLLQRFNVTTSAQSIEQLSTFLNERWKGVQPIAWGDALRESEFTRWVYQGLCGLEKEQKDAL

SEQ ID NO: 28 - DNA, 1260 bp, Escherichia coli
  CDS (1)..(1260)
atgcgctttgatactgtcattatgggcggcggcctcgccggattactctgtggcctgcaactgcaaaaacacggcctgcgctgtgccattgtcactcgtggtcaaagcgcactgcatttctcatccggatcgctggatttgctgagccatctgccagatggtcaaccggtgacagacattcacagtggactggaatctttgcgtcagcaggcaccagcccatccttactcccttctcgagccacaacgcgtgctcgatctcgcttgccaggcgcaggcattaatcgctgaaagcggtgcgcaattgcagggcagcgtagaacttgctcaccagcgggttacgccgctcggcactctgcgctctacctggctaagttcgccagaagtccccgtctggccgctgcccgcgaagaaaatatgtgtagtgggaattagcggcctgatggattttcaggcgcaccttgcggcagcttcgttgcgtgaactcggccttgccgttgaaaccgcagaaatagagctgccggaactggatgtgctgcgcaataacgccaccgaatttcgcgcggtgaatatcgcccgtttccttgataatgaagaaaactggccgctgttacttgatgcgcttattcctgtcgccaatacctgcgaaatgatcctgatgcccgcctgcttcggtctggccgatgacaaactgtggcgttggttgaatgaaaaactaccttgttcactgatgcttttgccaacgctgccgccttccgtgctgggcattcgtctgcaaaaccagttacagcgccagtttgtgcgccagggtggcgtgtggatgccgggcgatgaagtgaaaaaagtgacctgtaaaaatggcgtagtgaacgaaatctggacccgcaatcacgccgatattccgctacgtccacgtttcgcggttctcgccagcggcagtttctttagtggcggactggtagcggaacgtaacggcattcgagagccgattctcggccttgatgtgctacaaaccgccacgcggggtgaatggtataagggagatttttttgcgccgcaaccgtggcagcagttcggtgtaaccactgatgagacgctacgcccgtcacaggcagggcaaaccattgaaaacctgtttgccatcggttcggtgctgggcggatttgatcccatcgcccagggatgcggcggcggtgtttgtgccgtcagtgctttacatgccgctcaacagattgcccaacgcgcaggaggccaacaatga

SEQ ID NO: 29 - protein, 419 aa, Escherichia coli
MRFDTVIMGGGLAGLLCGLQLQKHGLRCAIVTRGQSALHFSSGSLDLLSHLPDGQPVTDIHSGLESLRQQAPAHPYSLLEPQRVLDLACQAQALIAESGAQLQGSVELAHQRVTPLGTLRSTWLSSPEVPVWPLPAKKICVVGISGLMDFQAHLAAASLRELGLAVETAEIELPELDVLRNNATEFRAVNIARFLDNEENWPLLLDALIPVANTCEMILMPACFGLADDKLWRWLNEKLPCSLMLLPTLPPSVLGIRLQNQLQRQFVRQGGVWMPGDEVKKVTCKNGVVNEIWTRNHADIPLRPRFAVLASGSFFSGGLVAERNGIREPILGLDVLQTATRGEWYKGDFFAPQPWQQFGVTTDETLRPSQAGQTIENLFAIGSVLGGFDPIAQGCGGGVCAVSALHAAQQIAQRAGGQQ

SEQ ID NO: 30 - DNA, 1191 bp, Escherichia coli
  CDS (1)..(1191)
atgaatgacaccagcttcgaaaactgcattaagtgcaccgtctgcaccaccgcctgcccggtgagccgggtgaatcccggttatccagggccaaaacaagccgggccggatggcgagcgtctgcgtttgaaagatggcgcactgtatgacgaggcgctgaaatattgcatcaactgcaaacgttgtgaagtcgcctgcccgtccgatgtgaagattggcgatattatccagcgcgcgcgggcgaaatatgacaccacgcgcccgtcgctgcgtaattttgtgttgagtcataccgacctgatgggtagcgtttccacgccgttcgcaccaatcgtcaacaccgctacctcgctgaaaccggtgcggcagctgcttgatgcggcgttaaaaatcgatcatcgccgcacgctaccgaaatactccttcggcacgttccgtcgctggtatcgcagcgtggcggctcagcaagcacaatataaagaccaggtcgctttctttcacggctgcttcgttaactacaaccatccgcagttaggtaaagatttaattaaagtgctcaacgcaatgggtaccggtgtacaactgctcagcaaagaaaaatgctgcggcgtaccgctaatcgccaacggctttaccgataaagcacgcaaacaggcaattacgaatgtagagtcgatccgcgaagctgtgggagtaaaaggcattccggtgattgccacctcctcaacctgtacatttgccctgcgcgacgaatacccggaagtgctgaatgtcgacaacaaaggcttgcgcgatcatatcgaactggcaacccgctggctgtggcgcaagctggacgaaggcaaaacgttaccgctgaaaccgctgccgctgaaagtggtttatcacactccgtgccatatggaaaaaatgggctggacgctctacaccctggagctgttgcgtaacatcccggggcttgagttaacggtgctggattcccagtgctgcggtattgcgggtacttacggtttcaaaaaagagaactaccccacctcacaagccatcggcgcaccactgttccgccagatagaagaaagcggcgcagatctggtggtcaccgactgcgaaacctgtaaatggcagattgagatgtccacaagtcttcgctgcgaacatccgattacgctactggcccaggcgctggcttaa

SEQ ID NO: 31 - protein, 396 aa, Escherichia coli
MNDTSFENCIKCTVCTTACPVSRVNPGYPGPKQAGPDGERLRLKDGALYDEALKYCINCKRCEVACPSDVKIGDIIQRARAKYDTTRPSLRNFVLSHTDLMGSVSTPFAPIVNTATSLKPVRQLLDAALKIDHRRTLPKYSFGTFRRWYRSVAAQQAQYKDQVAFFHGCFVNYNHPQLGKDLIKVLNAMGTGVQLLSKEKCCGVPLIANGFTDKARKQAITNVESIREAVGVKGIPVIATSSTCTFALRDEYPEVLNVDNKGLRDHIELATRWLWRKLDEGKTLPLKPLPLKVVYHTPCHMEKMGWTLYTLELLRNIPGLELTVLDSQCCGIAGTYGFKKENYPTSQAIGAPLFRQIEESGADLVVTDCETCKWQIEMSTSLRCEHPITLLAQALA

SEQ ID NO: 32 - DNA, 1506 bp, Escherichia coli
  CDS (1)..(1506)
atggaaaccaaagatctgattgtgatagggggcggcatcaatggtgctggtatcgcggcagacgccgctggacgcggtttatccgtgctgatgctggaggcgcaggatctcgcttgcgcgacctcttccgccagttcaaaactcattcacggtggcctgcgctaccttgagcactatgaattccgcctggtcagcgaggcgctggctgaacgtgaagtgctgctgaaaatggccccgcatatcgccttcccgatgcgttttcgcctgccacatcgtccgcatctgcgcccggcgtggatgattcgcattggtctgtttatgtacgatcatctgggtaaacgcaccagcttgccgggatcaactggtttgcgttttggcgcaaattcagtgttaaaaccggaaattaagcgcggattcgaatattctgactgttgggtagacgacgcccgtctggtactcgccaacgcccagatggtggtgcgtaaaggcggcgaagtgcttactcggactcgcgccacctctgctcgccgcgaaaacggcctgtggattgtggaagcggaagatatcgataccggcaaaaaatatagctggcaagcgcgcggcttggttaacgccaccggcccgtgggtgaaacagttcttcgacgacgggatgcatctgccttcgccttatggcattcgcctgatcaaaggcagccatattgtggtgccgcgcgtgcatacccagaagcaagcctacattctgcaaaacgaagataaacgtattgtgttcgtgatcccgtggatggacgagttttccatcatcggcactaccgatgtcgagtacaaaggcgatccgaaagcggtgaagattgaagagagtgaaatcaattacctgctgaatgtgtataacacgcactttaaaaagcagttaagccgtgacgatatcgtctggacctactccggtgtgcgtccgctgtgtgatgatgagtccgactcgccgcaggctattacccgtgattacacccttgatattcatgatgaaaatggcaaagcaccgctgctgtcggtattcggcggtaagctgaccacctaccgaaaactggcggaacatgcgctggaaaaactaacgccgtattatcagggtattggcccggcatggacgaaagagagtgtgctaccgggtggcgccattgaaggcgaccgcgacgattatgccgctcgcctgcgccgccgctatccgttcctgactgaatcgctggcgcgtcattacgctcgcacttacggcagcaacagcgagctgctgctcggcaatgcgggaacggtaagcgatctcggggaagatttcggtcatgagttctacgaagcggagctgaaatacctggtggatcacgaatgggtccgccgcgccgacgacgccctgtggcgtcgcacaaaacaaggcatgtggctaaatgcggatcaacaatctcgtgtgagtcagtggctggtggagtatacgcagcagaggttatcgctggcgtcgtaa

SEQ ID NO: 33 - protein, 501 aa, Escherichia coli
METKDLIVIGGGINGAGIAADAAGRGLSVLMLEAQDLACATSSASSKLIHGGLRYLEHYEFRLVSEALAEREVLLKMAPHIAFPMRFRLPHRPHLRPAWMIRIGLFMYDHLGKRTSLPGSTGLRFGANSVLKPEIKRGFEYSDCWVDDARLVLANAQMVVRKGGEVLTRTRATSARRENGLWIVEAEDIDTGKKYSWQARGLVNATGPWVKQFFDDGMHLPSPYGIRLIKGSHIVVPRVHTQKQAYILQNEDKRIVFVIPWMDEFSIIGTTDVEYKGDPKAVKIEESEINYLLNVYNTHFKKQLSRDDIVWTYSGVRPLCDDESDSPQAITRDYTLDIHDENGKAPLLSVFGGKLTTYRKLAEHALEKLTPYYQGIGPAWTKESVLPGGAIEGDRDDYAARLRRRYPFLTESLARHYARTYGSNSELLLGNAGTVSDLGEDFGHEFYEAELKYLVDHEWVRRADDALWRRTKQGMWLNADQQSRVSQWLVEYTQQRLSLAS

SEQ ID NO: 34 - DNA, 1071 bp, Escherichia coli
  CDS (1)..(1071)
atgaaaaaattgatcaatgatgtgcaagacgtactggacgaacaactggcaggactggcgaaagcgcatccatcgctgacactgcatcaggatccggtgtatgtcacccgagctgatgcccctgttgcaggaaaagtcgccctgctgtcgggtggcggcagcggacacgagccgatgcactgtggttatatcggtcaggggatgctttcgggggcctgtccgggcgaaattttcacctcaccgacgcccgataaaatctttgaatgcgccatgcaagttgatggcggcgaaggtgtactgttgattatcaaaaattacaccggcgatattcttaactttgaaacagcgaccgagttactgcacgatagcggcgtaaaagtgaccactgtggtcattgatgacgacgttgcggtaaaagacagtctttatactgccgggcgacgcggcgttgccaacaccgtattaattgaaaaactcgtaggcgcagcggcggagcgtggcgactcactggacgcctgtgcggaactggggcgtaagctgaataatcaaggccactcaataggtatcgctctcggtgcctgtaccgttcctgccgcgggcaaaccttcttttaccctggcggataatgagatggagtttggcgtcggcattcatggtgagccgggtattgaccgccgccccttctcttcccttgatcaaaccgtcgatgaaatgttcgacaccctgctggtaaatggctcataccatcgcactttgcgtttctgggattatcaacaaggcagttggcaggaagaacaacaaaccaaacaaccgctccagtctggcgatcgggtgattgcgctggttaacaatcttggcgcaactccgctttctgagctgtacggcgtctataaccgcctgaccacacgttgccagcaagcgggattgactatcgaacgtaatttaattggcgcgtactgcacctcactggatatgaccggtttctcaatcaccttactgaaagttgatgacgaaacgctggcactctgggacgccccggtccacaccccggcccttaactggggtaaataa

SEQ ID NO: 35 - protein, 356 aa, Escherichia coli
MKKLINDVQDVLDEQLAGLAKAHPSLTLHQDPVYVTRADAPVAGKVALLSGGGSGHEPMHCGYIGQGMLSGACPGEIFTSPTPDKIFECAMQVDGGEGVLLIIKNYTGDILNFETATELLHDSGVKVTTVVIDDDVAVKDSLYTAGRRGVANTVLIEKLVGAAAERGDSLDACAELGRKLNNQGHSIGIALGACTVPAAGKPSFTLADNEMEFGVGIHGEPGIDRRPFSSLDQTVDEMFDTLLVNGSYHRTLRFWDYQQGSWQEEQQTKQPLQSGDRVIALVNNLGATPLSELYGVYNRLTTRCQQAGLTIERNLIGAYCTSLDMTGFSITLLKVDDETLALWDAPVHTPALNWGK

SEQ ID NO: 36 - DNA, 633 bp, Escherichia coli
  CDS (1)..(633)
atgtcactgagcagaactcaaattgttaactggctcactcgttgtggcgatattttcagcaccgagagcgagtatcttaccggactggatcgcgaaattggcgatgctgaccacgggctaaatatgaaccgaggctttagcaaagtggtggaaaaactccctgctatcgcagataaagatatcggtttcattctcaagaataccggtatgacgctgctttccagcgtcggtggtgccagtggtccgctgttcggtaccttctttatccgcgccgcacaggcgacccaggcacggcaaagcctgacactggaagagctttatcagatgttccgcgatggcgcggacggcgtaatcagtcgcgggaaagccgaacctggcgataaaaccatgtgtgatgtgtgggtgccggtggtggaatcgttacgtcagtccagcgagcaaaatctctctgttccggtggcgctcgaagctgccagtagcatcgccgaatccgctgcacaaagtacgattacgatgcaagcccgcaaaggccgcgccagttatctcggtgaacgcagtattggtcaccaggatcccggcgcgacctcggtgatgtttatgatgcaaatgttggcgttagccgcaaaagagtaa

SEQ ID NO: 37 - protein, 210 aa, Escherichia coli
MSLSRTQIVNWLTRCGDIFSTESEYLTGLDREIGDADHGLNMNRGFSKVVEKLPAIADKDIGFILKNTGMTLLSSVGGASGPLFGTFFIRAAQATQARQSLTLEELYQMFRDGADGVISRGKAEPGDKTMCDVWVPVVESLRQSSEQNLSVPVALEAASSIAESAAQSTITMQARKGRASYLGERSIGHQDPGATSVMFMMQMLALAAKE

SEQ ID NO: 38 - DNA, 1419 bp, Escherichia coli
  CDS (1)..(1419)
atggtaaacctggtcatagtttcacatagcagccgactgggagaaggtgtcggtgaattagcccgtcagatgttaatgagtgatagttgtaaaatcgccattgccgcgggaattgacgatccacaaaatcccattggtaccgatgccgtcaaagtgatggaggccatcgaatctgttgctgatgccgaccatgtgctggtcatgatggatatgggtagcgcattattgagtgctgaaactgcgctggaattgctggctcccgagatcgccgcaaaagtacgtttgtgtgctgcgccgttggtcgaaggtacactggcagcaacggtcagcgcggcctcgggggcggatatcgacaaagttatctttgacgccatgcatgcgctggaagccaaacgtgaacaactgggtttaccgtcctccgacactgaaatctctgacacatgtcctgcgtacgatgaagaagcccgttctctggcggtggtcataaaaaaccgtaacggcctgcatgtacgtccggcctcccggctggtttataccttatcgacatttaatgccgatatgttgctggaaaaaaacggcaaatgcgtcacaccagagagtattaaccagattgcgttactacaagttcgctataacgatacgctgcgcctgattgcgaaagggccagaagctgaagaggcactgatcgctttccgtcagctggctgaagataactttggtgaaacggaggaagtcgctccacctactctgcgtcccgttccgcctgtttcgggtaaagccttttattatcaaccagttttatgtacggtacaggcaaaatcaaccctgaccgtggaagaagaacaagatcgattacgccaggctattgacttcacgttattagatctgatgacgttaacagcgaaagcagaagccagcgggcttgacgatattgccgcaatcttttctggtcaccatacactgttagatgatccggaactgctggcggcggcaagcgaactccttcagcatgaacattgcacggcagaatatgcctggcagcaagttcttaaagaacttagccagcaataccagcaactggatgatgaatatctacaagctcgctatattgatgtggacgatcttctgcatcgcaccctggtccacctgacccaaacgaaagaagaactcccgcagtttaactcgccaactattctactggcggagaacatttatccttccacagtactgcaactggatccggcggttgtaaaaggtatctgccttagcgccggaagtccggtatcccacagcgccctaatcgcccgtgaactggggattggctggatttgccagcagggtgagaaactgtatgcgatacaaccagaagaaacgctaacgctggacgttaaaacgcaacgtttcaaccgtcagggttaa

SEQ ID NO: 39 - protein, 472 aa, Escherichia coli
MVNLVIVSHSSRLGEGVGELARQMLMSDSCKIAIAAGIDDPQNPIGTDAVKVMEAIESVADADHVLVMMDMGSALLSAETALELLAPEIAAKVRLCAAPLVEGTLAATVSAASGADIDKVIFDAMHALEAKREQLGLPSSDTEISDTCPAYDEEARSLAVVIKNRNGLHVRPASRLVYTLSTFNADMLLEKNGKCVTPESINQIALLQVRYNDTLRLIAKGPEAEEALIAFRQLAEDNFGETEEVAPPTLRPVPPVSGKAFYYQPVLCTVQAKSTLTVEEEQDRLRQAIDFTLLDLMTLTAKAEASGLDDIAAIFSGHHTLLDDPELLAAASELLQHEHCTAEYAWQQVLKELSQQYQQLDDEYLQARYIDVDDLLHRTLVHLTQTKEELPQFNSPTILLAENIYPSTVLQLDPAVVKGICLSAGSPVSHSALIARELGIGWICQQGEKLYAIQPEETLTLDVKTQRFNRQG

SEQ ID NO: 40 - DNA, 1776 bp, Schizosaccharomyces pombe
  CDS (1)..(1776)
atgtctgtaaaacaatttgtatctgaaggacatatagtacgcccatacttgcttggacttgctagaagtaaccctggcctaactgttattgaacatgatcgtgtaatttacaggacagcctctgccccgggttctggtgatccgcctaaagtaactttagtgtctggtggtggtagtgggcacgagcctacacatgctggttttgttggtgatggagccttggacgccgtagcttgtggagacatttttgcttctccctctactaaacaaatttactctgctctcaaagctgttgcatctcctaagggaactttaattattgtcaaaaattacacaggtgatattattcattttggcttggcagctgagagagccaaggcagctggaatgaacgttgagcttgttgccgtaggagatgatgtttctgtaggcaaaaagagaggtgcactcgtaggacgaagaggtttaggagccactgtgctagtacacaaaattgcgggatctgcagcagcattaggactggatctgcatcaagttgctcaagtggcccaatcagtgattgacaatgctgctaccatagcagcttcattggatcactgcgccgttcccggtcgcaaatttgaaacaaacttgggtccagacgagtatgagattggaatgggtattcataacgagcctggaaccttcaaatcatcaccacttccatctattcccgagctagttacagagatgctttcgattttgtttggtgagaaaaacccggacaatagtttcgtagagttttcttcaaaagacgatgttattcttctagtaaacaatatgggtggtatgtccaacttagagttgggatatgctactgaagtagtttcggaacaattagccaaacgaggtatcattcctaaaagaaccatgtctggtacttttgtaaccgcattgaacggaccgggttttggaattacattggtcaatgcttctaaggctactccagatattttcaaatattttgacttgccaacaactgctagtggatggaacgtttcttatcataacgcaaaggactgggaggttttggctgacggcaaggtgccaacagctcccgctttggagcatacccgtaatgagaagcacagcggtgtaaaggctgacccaaagatgtttactaaaattttaaaagctgccgttgacgctatcaatgaatttgagccaaagacaacttggtacgatacgattgcaggagatggtgattgtggaacaacccttgtgaatggtggagaggccatcataaaggctattaatgataaatcaattcgattggatgatggtgtaaatggtattgacgatttggcctatattgttgaggattcgatgggaggtacatcgggtggtctttactcgatttatttgtctgcgctagccaaaggagttcgtgagtcaggcgattccgaattatccgtgcatacgtttgcgtttgcaagcaagtatgcacttgacgctcttttcaaatacactagggcccgtaaaggattccgtactctgatcgatgctattcaaccatttgttgaaactttaaatgaaggtaagggacttgatgctgctgcgaaagctgccacggaaggttctgaacaaactagaaaaatggatgctgtcgttggaagagcgtcttatgttgctaaagaggaacttcataagcttgatagtgagggaggcttaccggatcctggtgcctttgcgttagcggcgatcttgaaagcaattgttgaggctagtgaacattaa

SEQ ID NO: 41 - protein, 591 aa, Schizosaccharomyces pombe
MSVKQFVSEGHIVRPYLLGLARSNPGLTVIEHDRVIYRTASAPGSGDPPKVTLVSGGGSGHEPTHAGFVGDGALDAVACGDIFASPSTKQIYSALKAVASPKGTLIIVKNYTGDIIHFGLAAERAKAAGMNVELVAVGDDVSVGKKRGALVGRRGLGATVLVHKIAGSAAALGLDLHQVAQVAQSVIDNAATIAASLDHCAVPGRKFETNLGPDEYEIGMGIHNEPGTFKSSPLPSIPELVTEMLSILFGEKNPDNSFVEFSSKDDVILLVNNMGGMSNLELGYATEVVSEQLAKRGIIPKRTMSGTFVTALNGPGFGITLVNASKATPDIFKYFDLPTTASGWNVSYHNAKDWEVLADGKVPTAPALEHTRNEKHSGVKADPKMFTKILKAAVDAINEFEPKTTWYDTIAGDGDCGTTLVNGGEAIIKAINDKSIRLDDGVNGIDDLAYIVEDSMGGTSGGLYSIYLSALAKGVRESGDSELSVHTFAFASKYALDALFKYTRARKGFRTLIDAIQPFVETLNEGKGLDAAAKAATEGSEQTRKMDAVVGRASYVAKEELHKLDSEGGLPDPGAFALAAILKAIVEASEH

SEQ ID NO: 42 - DNA, 1830 bp, Pichia angusta
  CDS (1)..(1830)
atgtcgagcaaacactggaattacaagcaagacctggtccatgcgcacctcaaaggcctgtgtcatgccaatccagacctccaattcatcgaatccgagcgtgtggtgatcaacaagcactccaagcccgacaaggtgatgatcctatctggtggagggtctggccacgagccattgcacgctggctttgttggtgaaggctgtttggacgttggagtggctggttttgttttcgcctcgccctctacaaagcagattgtttcaggtttgaaggcaaagccctcagacaaaggtacgctaattgtggtgaaaaattacaccggcgacattcttcactttgggcttgctgcagagcgggccaaggccgaaggcgtccccgtggagctgctaattgtccaggacgacgtttctgtgggcagaaccaagaacggaatggtgggcagacgcggtctggccggtacgagtctggtgcacaagattgtcggtgccaaggccgccaaggactcgaacaaagcctcgttgagcgaggtgtaccagctgggcgaggccgtggtggccaatctggtgaccatcggcgcgtcgctcgaccactgcacaattccgggtaacagacaccacgagtccgagtccgacgacgaggacgagcagaaacatctgctcaaggaggacgagatcgaggtgggtatggggatccacaacgagtcgggcatcaagcgcgtttcgccaatcccgaccatcgacacgcttgtggcagacctgctcaagtacttgctcgacaagagcgacgaggagagacactatgtggacttcgactcgtcggacgaggttgtgctgatgatcaacaatttgggcggcacgtcgaacctcgagctgtacgctatccagaacactgttgttgagcagctggctaccgactacaagatcaagcccgcaagagtgtacacgggcgcgtacaccacgtcgctagacggtcctggtttttccatcacgttgctgaacgtgactcgggcggggggcaaggaggttttcgattgtctggactacccaaccaaggttcctgggtggaactcgtcgtacacaacggcagaatgggcggcgaaatccgagtcgttcgtcatcgacgctccgccagtgagcgacgcgtcggcgacctcgaaagtgcggttctcaagcagcacagtcaaggctgtgttggagagcggatgcaagaagttgctgaccaaggagccaaagatcacgctgtacgacacggttgcgggcgacggcgactgcggcgagacgctggccaacggcgcgcacgcgatcctggacctgctggctgccgacaagctggagatcaccgacggtgtccggagtctgacgcagatcactgacgtcgtcgaaacggctatgggaggcacctctggtgggctttactcgatctttatctctgcactcgccaagtcgttgaaggacagagagctccagcagggcggatacgaggtgacgccgcagatcctggctgcatcgctcaaggacgccctggagtcgctgtacagatacacacgggcccgtgctggcgaccggactctgatcgacgcgcttgcgccgtttgtggagcagtttgcggccagcaagggtgacctcaaccaggctaacaaggcgtgccacgagggagcagagtcaacgcgaaagctcaaggctaagtttggccgcgcgtcctacgtcagcgaggaggagttcaagccgtttgaggccgagggcgggctgccggatcccggcgccatcgggcttgctgcgctggtcgacggttttgccgaggcgtacagcaaaataggctccaacttgtag

SEQ ID NO: 43 - protein, 609 aa, Pichia angusta
MSSKHWNYKQDLVHAHLKGLCHANPDLQFIESERVVINKHSKPDKVMILSGGGSGHEPLHAGFVGEGCLDVGVAGFVFASPSTKQIVSGLKAKPSDKGTLIVVKNYTGDILHFGLAAERAKAEGVPVELLIVQDDVSVGRTKNGMVGRRGLAGTSLVHKIVGAKAAKDSNKASLSEVYQLGEAVVANLVTIGASLDHCTIPGNRHHESESDDEDEQKHLLKEDEIEVGMGIHNESGIKRVSPIPTIDTLVADLLKYLLDKSDEERHYVDFDSSDEVVLMINNLGGTSNLELYAIQNTVVEQLATDYKIKPARVYTGAYTTSLDGPGFSITLLNVTRAGGKEVFDCLDYPTKVPGWNSSYTTAEWAAKSESFVIDAPPVSDASATSKVRFSSSTVKAVLESGCKKLLTKEPKITLYDTVAGDGDCGETLANGAHAILDLLAADKLEITDGVRSLTQITDVVETAMGGTSGGLYSIFISALAKSLKDRELQQGGYEVTPQILAASLKDALESLYRYTRARAGDRTLIDALAPFVEQFAASKGDLNQANKACHEGAESTRKLKAKFGRASYVSEEEFKPFEAEGGLPDPGAIGLAALVDGFAEAYSKIGSNL

SEQ ID NO: 44 - DNA, 1827 bp, Pichia pastoris
  CDS (1)..(1827)
atgtctagtaaacattgggattacaagaaagatctggtactcagccatctggctggattatgtcagtccaacccccatgtaaggctaattgagtccgaaagagtcgtcatctccgccgagaaccaggaggacaagattaccttgatctctggtggaggttcaggacacgagcctctacatgctggctttgtcaccaaggacggtcttttggatgccgctgtggccgggttcatctttgcctcgccatctactaaacagatatttagcgcaatcaaagccaaaccttcgaagaagggaactttgatcatcgtcaagaactatacaggagacattcttcacttcggtcttgccgccgaaaaggccaaggccgaaggtctcaacgcagaactgctgattgttcaggacgatgtctctgttggtaaagccaagaatggcctggtagggcgtagaggtttggctggaacttctttagtgcacaagattttgggtgccaaagcttatttacaaaaggacaacctggaattgcatcagctggtcactttcggtgagaaggtcgttgccaacctggttacaattggtgcttccttggaccatgtcaccattccagccagagcaaacaaacaggaggaagatgattcagatgacgagcacggctacgaagtgctaaagcacgatgaatttgaaatcggaatgggtattcacaacgaaccaggtatcaagaagtcgtcgccaattccaacagtagacgaattggtggctgagctactggagtaccttctttcgactaccgataaggaccgaaattacgtccaatttgacaagaatgatgaagttgtgctactgatcaataacttgggaggaacctcagtgttggaattgtacgctattcagaacattgttgtcgaccaactggcctccaaatattccattaaaccagtccgcattttcactggcactttcaccacatcactggatgggcccggtttttctatcactttattgaatgctaccaagacaggagataaagatatcctaaagtttttagaccacaagactagtgcccctggctggaactccaacatatcagactggtctggcagggttgacaattttattgtggctgccccagaaatagacgaaggagacagttcatctaaggtatctgttgatgctaagctgtatgctgaccttctggagtctggtgtgaagaaggtcatttcaaaagagccaaagatcacattgtatgacactgttgcaggagatggtgattgtggtgaaacgttggcgaatggttccaatgccattttaaaggctctggctgaaggaaagcttgatctcaaagacggtgttaaatcactggtgcaaattacagacatcgtggaaactgccatgggtggaacatctggaggtctgtactccattttcatcagcgctttagcaaagtctttgaaagagaaggaactttccgagggtgcctacacactgacccttgaaacgatctctggatccttacaggcagcacttcagtctctattcaaatacacaagagcacgtactggagacagaactcttattgacgctttggagccatttgtaaaggaattcgccaagtctaaggatctgaaacttgcaaacaaagctgcccatgatggtgctgaagccacaagaaagctcgaagccaagtttggaagagcatcttatgttgccgaagaagagttcaaacaattcgaaagtgaaggtggactacctgatcctggtgctattggtcttgccgctttgatttcaggaattactgatgcatacttcaaatctgaaaccaagttgtag

SEQ ID NO: 45 - protein, 608 aa, Pichia pastoris
MSSKHWDYKKDLVLSHLAGLCQSNPHVRLIESERVVISAENQEDKITLISGGGSGHEPLHAGFVTKDGLLDAAVAGFIFASPSTKQIFSAIKAKPSKKGTLIIVKNYTGDILHFGLAAEKAKAEGLNAELLIVQDDVSVGKAKNGLVGRRGLAGTSLVHKILGAKAYLQKDNLELHQLVTFGEKVVANLVTIGASLDHVTIPARANKQEEDDSDDEHGYEVLKHDEFEIGMGIHNEPGIKKSSPIPTVDELVAELLEYLLSTTDKDRNYVQFDKNDEVVLLINNLGGTSVLELYAIQNIVVDQLASKYSIKPVRIFTGTFTTSLDGPGFSITLLNATKTGDKDILKFLDHKTSAPGWNSNISDWSGRVDNFIVAAPEIDEGDSSSKVSVDAKLYADLLESGVKKVISKEPKITLYDTVAGDGDCGETLANGSNAILKALAEGKLDLKDGVKSLVQITDIVETAMGGTSGGLYSIFISALAKSLKEKELSEGAYTLTLETISGSLQAALQSLFKYTRARTGDRTLIDALEPFVKEFAKSKDLKLANKAAHDGAEATRKLEAKFGRASYVAEEEFKQFESEGGLPDPGAIGLAALISGITDAYFKSETKL

SEQ ID NO: 46 - DNA, 1824 bp, Debaryomyces hansenii
  CDS (1)..(1824)
atgtcgttagctaaacactggggatactcaaaggatttggttttggaaaatttaaagggtttggctgctgcaaaccccaaaatttctcttataccagcagaaaaaacagtcgtgtataatgattccttacaatctaaaggaaacaagaatattaatcgtataatggttatttctggaggaggttcaggccatgaaccgttacatgcgggatttgttggtgtaaatgcattagatgctgctgtatctggttcaatatttgcctccccttcagccaagcagatttttgcagccattaagtcaattagctccaaacaaaacaattctaaaggtactttggtaattgtcaaaaattatacaggagatgttctacattttggacttgctgtcgaaagagcaaaagcacatggctacaaaatagacatgataattgttggtgatgacgcagctgtaggtaggtcaaagggtggaatggttggaagaagagcattggcagccactgcattggttcacaaaattgttggatctgctgcttctgaaattgaagatctaagcagacttaaaatattgggtgattccgttgcgaataatacggtgaccattggtgccaccttagatcattgctctgttcctgggcgtgatattgcaaactttgagcctattggccagaatgatgctgaaataggtctagggatacataacgaaacttcggtcaagaaggtaaaccctgtaccaatgatagactcattagttcaggacttattagaattcctattaaacgaaaatgacaaagatcgttactttgtaccatttgatctcagtaatgatgaaacggtcttgcttgtcaacaatctcgggggtacatctactttagaaatgtatgctattacaaattgcgttatcgaaacattataccaacaatacagtttgagaccaaaaaaagtgatagtgggagaatttgctacgtctttaaatgcccctgggttttcgattaccttacttaacgtctcttgtgcatctaaacaatcccaaatttccatttcacatataatgagttacttggatttgccaacagatgcccctggttggaaggcacatccgtgtgggtttggacttgaaagagacatcaatattgagacatcaatcaatggtattgattcttttgtcaagtcacaattaaagctttccagagaacaacagacggactttagaagcagtctagttaatgggttggaaaaattattagacaaagaaccaagcattacattttatgatactgttgctggtgatggtgactgtggtgaaaccttagcgtctggtgcaaatggaatattggaatcattaaggaacaacgaaatctgctttgaagatccagtttattccatatctcaaatagcaaacattgtagaggataaaatgggcggaacttcaggaggcttatattcaattttcttaacctcgttgataaaacaccttcaagattgtactacattgaacttatgtgaaatgtttgctagttctttgcataatgcgctatatcagggcttatataaatacactagggcacgagtgggtggaagaactttgattgatgctttagagccgtttgtgaataccttcaacgacactctaaatttctcaaaggctgcccaggctgctatcgatggatctgaatcgactaggaaattagctgcaaaatttgggagagcgtcttatgtcaatgaacaagaatttaaacagttcgacgaagaaggaggcttaccggatccgggagctattggattagccaccttaattgctgggtttgccggtgttgactataattaa

SEQ ID NO: 47 - protein, 607 aa, Debaryomyces hansenii
MSLAKHWGYSKDLVLENLKGLAAANPKISLIPAEKTVVYNDSLQSKGNKNINRIMVISGGGSGHEPLHAGFVGVNALDAAVSGSIFASPSAKQIFAAIKSISSKQNNSKGTLVIVKNYTGDVLHFGLAVERAKAHGYKIDMIIVGDDAAVGRSKGGMVGRRALAATALVHKIVGSAASEIEDLSRLKILGDSVANNTVTIGATLDHCSVPGRDIANFEPIGQNDAEIGLGIHNETSVKKVNPVPMIDSLVQDLLEFLLNENDKDRYFVPFDLSNDETVLLVNNLGGTSTLEMYAITNCVIETLYQQYSLRPKKVIVGEFATSLNAPGFSITLLNVSCASKQSQISISHIMSYLDLPTDAPGWKAHPCGFGLERDINIETSINGIDSFVKSQLKLSREQQTDFRSSLVNGLEKLLDKEPSITFYDTVAGDGDCGETLASGANGILESLRNNEICFEDPVYSISQIANIVEDKMGGTSGGLYSIFLTSLIKHLQDCTTLNLCEMFASSLHNALYQGLYKYTRARVGGRTLIDALEPFVNTFNDTLNFSKAAQAAIDGSESTRKLAAKFGRASYVNEQEFKQFDEEGGLPDPGAIGLATLIAGFAGVDYN

SEQ ID NO: 48 - DNA, 1752 bp, Escherichia blattae
  CDS (1)..(1752)
atggtctgcccgccagaccttaactcacaaccttttacttcccgacacacgcgctacagcccgctgtacgggtgtgttgtacctaacgatcaggaaaccgttatgtcgcaatttttttataaccaacgcgaaaatcttgtcagtgacgccatcgaaggggcaatgattgccagcccgtggaacaacctggcccgtctggagagcgatcccgccatccgcatcgtggtgcgccgggatctggataaatcccgggtggcggtgatctccgggggcggtgccggtcacgaaccggcccatgtgggctttgtgggtaaaggcatgctgaccgccgccgtgtgtggtgatctgtttgcctcaccgagcgtggatgcggtgcttaccgccattcaggcggtcaccggcgaggcgggctgcctgctgattgtcaaaaactacaccggtgaccggctgaacttcgggctggcagcagagaaagcccggcgcatgggctacaaagtggacatggtgattgtgggggatgatatctcactgccggagaacaaacacccgcgcggcattgccgggacgattatgatccacaaagtggcggggtacttcgccgaaaccggctgcaacctggacacggtcgcccgggaagcccggctggcaatggagcgggtattcagtattggcgtggccctttccagctgccacttaccggccgatccgcaggatggcgtgcgccatcacccgggccaggctgagctgggcatggggatccacggggagcccggcgcaagcgtcatcgacacccagaacagcaccgacattgtgcgcctgatggtggcaaaaatccgcgctgccctgcctgaaaccggccgcctgctgctgatgctgaataaccttggcggcgtctcagtcaccgaaatggcgatcctcacccgggagctggcccactgtgagcttgccacccgcaccgactggttgatgggcccggcaccgctggtcagcgccctggacatgaaaggcttttccatcaccgccctggtcatggaagagagcattgaaaaagccctgctggcagacgtggaaaccgcaggctggctgccgccagtgcgcctgcgcgccagccagacccagccctgcaatatccgcagtgcccgggtggcattcaccccgtcagacaacccggtagtgggccagtatgtggagaccgtcaccgccacactcagcgcccaggaagcggaacttaacgccctggatgccaaagtgggcgacggcgataccggctccacctttgccgccggggcccgcgccattgcagaactgctgcaccagcaccagctgccgctcagccagctggataccctgtgcgccctgatcggcgaacgcttaacagtagtgatggggggctccagcggtgtgctgatgtccatcttctttacggccgcaggccaggcaatcagcgaagggaaaccggtggtggccgcactccaggcgggcctggcgcagatgaagtattacggcggtgccgatctgggtgaccgcaccctgattgatgccctgcaacctgcgctggcagctctggcacagcatccgggggatctggcggcggcataccaggcagcccgggacggggcagacgccaccacccgcgccaccaaagccaatgccggtcgcgcctcttacctgaacagcgacagcctggcgggcaatatggacccgggcgctcacgcagtcgcaatggtctttaaggcgctggcgggcgcctga

SEQ ID NO: 49 - protein, 583 aa, Escherichia blattae
MVCPPDLNSQPFTSRHTRYSPLYGCVVPNDQETVMSQFFYNQRENLVSDAIEGAMIASPWNNLARLESDPAIRIVVRRDLDKSRVAVISGGGAGHEPAHVGFVGKGMLTAAVCGDLFASPSVDAVLTAIQAVTGEAGCLLIVKNYTGDRLNFGLAAEKARRMGYKVDMVIVGDDISLPENKHPRGIAGTIMIHKVAGYFAETGCNLDTVAREARLAMERVFSIGVALSSCHLPADPQDGVRHHPGQAELGMGIHGEPGASVIDTQNSTDIVRLMVAKIRAALPETGRLLLMLNNLGGVSVTEMAILTRELAHCELATRTDWLMGPAPLVSALDMKGFSITALVMEESIEKALLADVETAGWLPPVRLRASQTQPCNIRSARVAFTPSDNPVVGQYVETVTATLSAQEAELNALDAKVGDGDTGSTFAAGARAIAELLHQHQLPLSQLDTLCALIGERLTVVMGGSSGVLMSIFFTAAGQAISEGKPVVAALQAGLAQMKYYGGADLGDRTLIDALQPALAALAQHPGDLAAAYQAARDGADATTRATKANAGRASYLNSDSLAGNMDPGAHAVAMVFKALAGA

SEQ ID NO: 50 - DNA, 1647 bp, Enterobacter sp.638
  CDS (1)..(1647)
atgtccagattcttttttaatgaccgcaaacagctggtcaacgacgccattgaaggcatactgatttccgcgccgcacgggaatcttgtcaaacttgatatcgatccggccattcgggtggttgcgcgtagcgactgggataaaagccgcgtagcggtgatttccggtggtgggtcggggcacgaacccgctcatgccggatttgtcggcaaagggatgttgaccgcagccgtctgtggcgatctgtttgcctcaccgagcgtagatgcggtgttaaacgcgattgtggcggtaacgggcgatcgcggttgcctgttaatcgtcaaaaattataccggcgatcggcttaactttggcctcgcggcggaaaaggccaaacgctatgggctgaaggttgagatggtgattgttgctgatgacatcgccctgccggataacaaacagccgcgtggcattgcgggtacggcgctggtacacaaaattgccggatatgcagccgaacaggggaaatcactggctgacgtgcgggatattgcgcagcaggcctgtgacaatatctggagcctgggcgtggcgatgcaaacgtgcaacctgccgggcagcgacgatgaagaagggcgtatcaaggatggacatgtcgaactggggctgggcattcacggcgagccgggcgcgtcggtggttgatacgcacaacagcaaagagattatcgacaccctggtgaagccgttaaaagagacggccggcgaaggcaaatttgcggtgctgattaacaatctcggcggtgtatcggcgctggagatggcgctgctcacgaaagaactggcggattctgcgctgaaagaaaatattgcgtatctgattggccctgcgccgctggtaagctcgctggatatgaaaggcttttcgctgtcactgttacagcttaacgatacctttgagaaagccattaacgcacccgtcgaaactatcggctggcaaaagccggtagcattcgcgccattacgcacgctttcgcatactgcgattcaggatcgtgttgaatttacgccttccgggaacgacgaggtcgcagcgcgagtggcagcggcgacgcaaacgttgctcgctctggagaaccgtttaaatgcgctggacgccaaagtgggcgacggcgataccgggtcgacttttgcgcaaggcgcgcgggaaattgcgcagcttctggagcaaaaacagcttccgctaaacgatctttctaagctgctgttgttgatcggcgaacggctggcgacggtcatgggcgggtcgagtggcgtcctgatgtcgatcttcttcacagctgccggacagaaaatgcatgacggaaaatcactgccggaggcattgctgagtgggcttgcgcaaatgaagcattacggcggagcggatcttggcgatcgtaccttgatcgacgcgctacagcctgcactggagacgctgcataacggcgatattcaggcggctgcccaggcagcgaaaaaaggcgcagacgctacggctggcatgcaaaaagcgggagcagggcgttcgtcgtatgtgaataaagagaacctggaaggtgtaatagatcctggggcagtggccgttgcagaggtgtttgcggcagtggccaaagcaaaacagtag

SEQ ID NO: 51 - protein, 548 aa, Enterobacter sp.638
MSRFFFNDRKQLVNDAIEGILISAPHGNLVKLDIDPAIRVVARSDWDKSRVAVISGGGSGHEPAHAGFVGKGMLTAAVCGDLFASPSVDAVLNAIVAVTGDRGCLLIVKNYTGDRLNFGLAAEKAKRYGLKVEMVIVADDIALPDNKQPRGIAGTALVHKIAGYAAEQGKSLADVRDIAQQACDNIWSLGVAMQTCNLPGSDDEEGRIKDGHVELGLGIHGEPGASVVDTHNSKEIIDTLVKPLKETAGEGKFAVLINNLGGVSALEMALLTKELADSALKENIAYLIGPAPLVSSLDMKGFSLSLLQLNDTFEKAINAPVETIGWQKPVAFAPLRTLSHTAIQDRVEFTPSGNDEVAARVAAATQTLLALENRLNALDAKVGDGDTGSTFAQGAREIAQLLEQKQLPLNDLSKLLLLIGERLATVMGGSSGVLMSIFFTAAGQKMHDGKSLPEALLSGLAQMKHYGGADLGDRTLIDALQPALETLHNGDIQAAAQAAKKGADATAGMQKAGAGRSSYVNKENLEGVIDPGAVAVAEVFAAVAKAKQ

SEQ ID NO: 52 - DNA, 1695 bp, Psychromonas sp. CNPT3
  CDS (1)..(1695)
atggttatattattttttaacctactttattttaaactactttttggagtaattatgagccgtttatttattaatgataaagcaacgttagtccatgatgcgattgatggcatcttatatagcaataagcataataaccttgttcgtttggatgttgatccgcaaattagaattgtgacgcgtaatgattggcatcacgataaagttgccattatctctggtggtggctctgggcatgagcctgctcatgttggttttattggtaaaggtatgttaacggctgctgtttgtggcgatgtttttgcctcaccaagtgtggatgcggtgttaaatgccatagtccatgtcacaggagagaaaggttgtttggtcattgttaaaaactataccggcgaccgtttgaactttggccttgcttgtgaaaaagccaaaaaaatgggtttaaacgttgaaatggtaatagtagacgatgatatctctattcctgacaatcttaaacctcgtggtattgctggcacattgtttgtccataaagtggcaggtaatgcagcagagcaaggcgcttctttaaatgtcgttaaaaaggcagcccaaggtgcgattgatgcaacggcaagtattggcctcgcactgacaagttgctctttaccgggagaagaatcaacgcagcgtattgcagaaggcaaggctgaattagggttaggtattcatggagagcctggcattaaaacaattgatgtaacatgttgtcgtgatcttgtgatgatcatggttgataaactaaaacaatcgttttctgctccagatattaaaattgcagtgatgatcaacaatttaggtggcgtttctccattagagatgagccttatatgtaaagacatcgtggaatctgagttaaaaaataatattgagttagtggttgggcctgctccatttatgacggctattgatatgaaaggattttcaatttcagtgattgaattaacaggcgatcatgctcaagctctgtgcgcccctgttgaagtggatgcatgggttgaagccattccattgcgcccattaaatgtgataaaaaaagataaagtgtccattaactttgcatttgaagcgtctgaaaatgcacaggttgcaagtattgttaaaacagtaaccttggctttgattaatgcagaaaaagagttaaatcgtctggatacgttagtgggcgatggggataccggttcaacgttctctgcgggtgctcgacaagttttagctgagctcaatgcgggtaatttaccacttaatgatactggcgcgttgcttaatgtcattggggaacaacttgctaccgttatggggggatcgtcaggcgtattattctctatcttcttcacagcagcgggtcatcattaccagcaacatggtgatacagtacaagcattacaagccggtttacaacaaatgatgcaatacggtggagcaaaaccgggtgatcgtacgatgattgatgcaatgtacccagcctttatcgcttggaaaaatgaaggttttgaagctgccattgttgcggctaaaataggggcagaaagcacagccactatggtcgaagccaaagcaggacgctcttcttatttaaacagtgagagtttaaaaggtgttaaagatccgggctcagtcgctgttgagttggtttttgacgcgtttaatgtgtaa

SEQ ID NO: 53 - protein, 564 aa, Psychromonas sp. CNPT3
MVILFFNLLYFKLLFGVIMSRLFINDKATLVHDAIDGILYSNKHNNLVRLDVDPQIRIVTRNDWHHDKVAIISGGGSGHEPAHVGFIGKGMLTAAVCGDVFASPSVDAVLNAIVHVTGEKGCLVIVKNYTGDRLNFGLACEKAKKMGLNVEMVIVDDDISIPDNLKPRGIAGTLFVHKVAGNAAEQGASLNVVKKAAQGAIDATASIGLALTSCSLPGEESTQRIAEGKAELGLGIHGEPGIKTIDVTCCRDLVMIMVDKLKQSFSAPDIKIAVMINNLGGVSPLEMSLICKDIVESELKNNIELVVGPAPFMTAIDMKGFSISVIELTGDHAQALCAPVEVDAWVEAIPLRPLNVIKKDKVSINFAFEASENAQVASIVKTVTLALINAEKELNRLDTLVGDGDTGSTFSAGARQVLAELNAGNLPLNDTGALLNVIGEQLATVMGGSSGVLFSIFFTAAGHHYQQHGDTVQALQAGLQQMMQYGGAKPGDRTMIDAMYPAFIAWKNEGFEAAIVAAKIGAESTATMVEAKAGRSSYLNSESLKGVKDPGSVAVELVFDAFNV

SEQ ID NO: 54 - DNA, 1647 bp, Stapia aggregata IAM12614
  CDS (1)..(1647)
atgaagcaattcatcaataccaaggaaacgctcgtcaccgaagcgattgacggcatgttgcgcacggccggcgggcggcttgcccggcttgacggctatccgcatatcaaggtggtcgtgcgcaccgactgggacaaatcgaaggtggctctggtgtccggcggcggttccggccacgagccgagccatgccggtttcgtcggccagggcatgctgacggcggccgtctgcggcgaagtgtttgcctcgccttccgtggatgcggtgctggcgggcattctggccgtcaccggcaaggctggctgcctgctgatcgtcaagaactacaccggcgaccggctgaacttcggtctggccgccgagcgggcccgctccttcggactgaaggtcaacatggtgatcgtcgacgacgacgttgccctgccggacctgccgcaggcgcgcggtgtcgccggcacgctgttcgtgcacaagatcgccggagcgcttgccgatcagggtgcggatctggaaaccatcacggaggccgccaggaaaaccatcggcggtgcgatttccatcggcatgtcgctggacacctgcacggtgccgggatcgcccaaggaagaccgcattgcacacggcaaggcggaactcggacttggcattcacggcgaggcggggatcgagcaggtcgactattccaacgcccgcgcggccatggccatggtggtggaccggctggcgccgaacctctcgcccggaccgcatgtggcgatcctcaacaatctgggcagcacgacgccgctggaaatgtcggtgcttctggaagaactcacggcttcgcgcatcggcagccagatccgctgggtcatcggcccggcggcgatgatgacctcgctcgacatgcatgggttctccgtgtcgctgctgccggtcggcaagaccgaagaagccttgctgcaggccccggtcgcgccctgggcatggcccggctgccttgcgcttggcgcagtgtccgtgctgccgctaccggacggcctgacgccgatccagccgctgccgtccaagaacccggagacgcggaagttcatcgagcgctgctgcgatatcctgatcgccgccgaggacgacctcaatgcgctcgacgccaagtcgggcgacggcgacaccggcagcacgcttgccaccgccgcccgggcgctggtgaaggcgctcgaccggctgccgctggcggatctcacccagctctaccgggccatcgggctggaactcagccagaccatgggcgggtcttccggggtgctgctggcgatcttctttgccgctgcgggcgatgcgtcgtccagcgggcgcggggccatcggagccctgaaggccggtcttgaccggatcatgcaggtgggcggcgcccagccgggcgaccgcaccatgatcgacgcgctgctgccggcactgaatgcgctggaaaacggtatcgaggctgcggcgagcgaggcccgtcagggggcggatgcgacgtcgcggatcacgcgggcacgcgcgggcagggcgtcttatgtctccgaggccagcctctccggacacaacgatccgggcgcggaagccgttgcgcggctgttcgagcaattgaccctttctccggcccttacctccaagtccgcatag

SEQ ID NO: 55 - protein, 548 aa, Stapia aggregata IAM12614
MKQFINTKETLVTEAIDGMLRTAGGRLARLDGYPHIKVVVRTDWDKSKVALVSGGGSGHEPSHAGFVGQGMLTAAVCGEVFASPSVDAVLAGILAVTGKAGCLLIVKNYTGDRLNFGLAAERARSFGLKVNMVIVDDDVALPDLPQARGVAGTLFVHKIAGALADQGADLETITEAARKTIGGAISIGMSLDTCTVPGSPKEDRIAHGKAELGLGIHGEAGIEQVDYSNARAAMAMVVDRLAPNLSPGPHVAILNNLGSTTPLEMSVLLEELTASRIGSQIRWVIGPAAMMTSLDMHGFSVSLLPVGKTEEALLQAPVAPWAWPGCLALGAVSVLPLPDGLTPIQPLPSKNPETRKFIERCCDILIAAEDDLNALDAKSGDGDTGSTLATAARALVKALDRLPLADLTQLYRAIGLELSQTMGGSSGVLLAIFFAAAGDASSSGRGAIGALKAGLDRIMQVGGAQPGDRTMIDALLPALNALENGIEAAASEARQGADATSRITRARAGRASYVSEASLSGHNDPGAEAVARLFEQLTLSPALTSKSA

SEQ ID NO: 56 - DNA, 1641 bp, Rhizobium leguminosarum
  CDS (1)..(1641)
atgaaacacttcttcaaccgcagggaaaacatcgtcaccgaagccttggacggtctgcttctgacgagcagcaagggtcgtcttgcccgcctcgacagctttcccgacatcaaggtgatcctgcgcgctgactgggacaagtcgaaggtggcgatcatctcaggcggcggcgccggtcatgagccctcccatgccggcttcgtcggtaagggcatgctgacggctgccgtatccggcgagattttcgcctcgccgagcgtcgatgccgtgctgacagcgatccgcgccgtcgccggcgaaaagggcgccttgctgatcgtcaagaactataccggcgaccggctgaatttcggcctcgccgccgagaaggcgcgcgccgaaggtttcgacgtcgaaatggtcatcgtcgccgacgatatcgccatccccgagatcaaccagccgcgcggcgtcgccgggactctgttcgtccacaagatcgctggctatcacgccgaaaggggcgaggacctgaagacggtcgcagcccatgccgcggcagcggccggcgacatcgtctcgctcggcatgtctctgtccacctgcagcgtgcccggccaggcgcatgagagccgcctcggcgagaacgagggcgaactcggtctcggcatccatggcgagcccggcgtcgagcgcattgcgctgcagccggtcgtcgatatcgtcgccaccatggtggcgcgcctatcgcctgcgctgcgcgaagggggaaaccacgcccttctcatcaacaatctcggcgccgtaccgccgctcgaaatgaccgttattgccaatgtggtgctgtcctcgtcgcttgccgatcgcgtcaggctgatcatcggcccggcgccgatgatgaccgcgctcaacatgaacggcttctcgctgtcgctgatccgactggatgccgctcgcgaggcggcgctgacggcagcggtcgaaccgcatgcctggatgccagccgtcgaacgccacgagatcagggtcatcgccgcaccgcgaacatcagccggactgaacggcgcgccagtggccggggataatctccgcaaccggcgtctgatcacagcgctctgcgagcatctgatctcgcaggaaagcgaactcaaccggctggatggccgcgtcggcgacggtgataccggctcgacggtggcgacaggcgcccgcagcgtgcttgcccgcctggacacgctgccgcttgatcggccggctgcaacgcttgcctcgctcggcgacatcctcggcaccagcatgggcggatcgagcggcgtgctgctgtcgatcttcttcaccgcagcggcaaaggcgatggccgacaaggccgatatatcagcagcccttattgccgggctcgacaggatgacgttctatggcggagccgaagtcggcgaccggacgatggtcgatgcgctgtcgcctgccctgcaggcgctcgcatccggcgatgtcgcggcagcggccagggctgctgccgcaggtgcggagtcgacgaagacgatgatgaaagcgagagccggccgcgcctcctatgtcggcgaaagggatctggcaggtgtcgctgatcccggcgccgtcgcggttgccggcgcgttcggtgtggcggcaagcctcgcctga

SEQ ID NO: 57 - protein, 546 aa, Rhizobium leguminosarum
MKHFFNRRENIVTEALDGLLLTSSKGRLARLDSFPDIKVILRADWDKSKVAIISGGGAGHEPSHAGFVGKGMLTAAVSGEIFASPSVDAVLTAIRAVAGEKGALLIVKNYTGDRLNFGLAAEKARAEGFDVEMVIVADDIAIPEINQPRGVAGTLFVHKIAGYHAERGEDLKTVAAHAAAAAGDIVSLGMSLSTCSVPGQAHESRLGENEGELGLGIHGEPGVERIALQPVVDIVATMVARLSPALREGGNHALLINNLGAVPPLEMTVIANVVLSSSLADRVRLIIGPAPMMTALNMNGFSLSLIRLDAAREAALTAAVEPHAWMPAVERHEIRVIAAPRTSAGLNGAPVAGDNLRNRRLITALCEHLISQESELNRLDGRVGDGDTGSTVATGARSVLARLDTLPLDRPAATLASLGDILGTSMGGSSGVLLSIFFTAAAKAMADKADISAALIAGLDRMTFYGGAEVGDRTMVDALSPALQALASGDVAAAARAAAAGAESTKTMMKARAGRASYVGERDLAGVADPGAVAVAGAFGVAASLA

SEQ ID NO: 58 - DNA, 1701 bp, Myxococcus xanthus
  CDS (1)..(1701)
atgaagaagctggtcaacgcccctcgcgcggtggtgcgggagatgctggaggggttggtctcgctcgcccccgggcaggtgctgctggacggggagtcggtggtgctccgcgccgacacgccttccgacgtccgcgcgcgcaaggtggctgtcatctccggtggcggcagcggccatgagccggcgcacgcgggctacgtgggcgcgggcatgctggacgcggcggtggccggtgacgtcttcacctcgcccagcaccgatgccgtgctggccgccatccgcgccgtcgcggggcccgcgggcgcgctgctcgtcgtgaagaactacaccggggaccggctcaacttcgggctcgccgccgagctggcgcgcgccgagggcatccccgtggagacggtggtggtggcggacgacgtgtccctgcacgacacggtggagcccgcgcggcgccggggcatcgctggcacggtgctggtccacaaggtcgcgggcgcggcggccgaggcgggcgcggcgctccaggacgtcctccgcgaggccaccgcggcggcggaggtgctgggcaccatgggcgtggccctggggccctgcaccgtgcccgcggcgggcaagccgggcttcacgctggaggaggacgaaatcgagctgggcctgggcatccacggcgagcagggcgtgcggcgcgtgccgatgcagacggcggacagcctggtggacacgctgctcaccaccatcgtcgaggaccggcgcatcacctcgggagacagggtggtgctggtggtcaacggattgggcggcacgccgcccatggagctggccatcgtcgcccggcgcgcactggccgctctgcgtcagggcggcatccgcgtggagcgcgcgtggagcgggacgttcctctccgcgctggagatgcccggctgctcgttgacgctgctgaaggtggacgacgcgcggctggcccgcctggatgcggcggtggatgcgcccgcgtggcccggcgcgggacggctgccgaaggagccgggggtgtaccggccttcgtccacggcgtctccagcatcgcttccggcggaggcgccgcaaccggggatggaccgcttccggaaggccgccttgcgggtggcggacgcattcgagcagtcggagccccggctgaccgcgctcgatagcgccgcgggcgacggtgacctgggcctcagtctggtgcgtggcgccgaggcgattcgcgctcttccggaggacgcgtggacgagccccgcgcgtgcgctgacggccattggcaatgccttgcggcgcagcattggcggcagctcggggcccttctacgcgacggcgctgctgcgcgccgcgcgcaggctggcggaagggcccgtggatgccgccgcatgggccgaggccttcgacctcgccgtcaccgccgtatcggagctgggcggcgcgcggcctggggaccgcaccatgctcgatgcactccggcccgccgccgacgccttcgcgaaggcggtgcgttgcgggcagggggctcgcgaggcctgggcctcggcggtgcacgcggccgaggcgggagaggaggcgacgtcccggatgcagccgcgcctgggacgcgccagctacctgggtgcgcgcgccgtcggtgtgccggacgcgggcgccgcggccgtggtggtgtggatgaaggcgctcacgcctggcatcggctga

SEQ ID NO: 59 - protein, 566 aa, Myxococcus xanthus
MKKLVNAPRAVVREMLEGLVSLAPGQVLLDGESVVLRADTPSDVRARKVAVISGGGSGHEPAHAGYVGAGMLDAAVAGDVFTSPSTDAVLAAIRAVAGPAGALLVVKNYTGDRLNFGLAAELARAEGIPVETVVVADDVSLHDTVEPARRRGIAGTVLVHKVAGAAAEAGAALQDVLREATAAAEVLGTMGVALGPCTVPAAGKPGFTLEEDEIELGLGIHGEQGVRRVPMQTADSLVDTLLTTIVEDRRITSGDRVVLVVNGLGGTPPMELAIVARRALAALRQGGIRVERAWSGTFLSALEMPGCSLTLLKVDDARLARLDAAVDAPAWPGAGRLPKEPGVYRPSSTASPASLPAEAPQPGMDRFRKAALRVADAFEQSEPRLTALDSAAGDGDLGLSLVRGAEAIRALPEDAWTSPARALTAIGNALRRSIGGSSGPFYATALLRAARRLAEGPVDAAAWAEAFDLAVTAVSELGGARPGDRTMLDALRPAADAFAKAVRCGQGAREAWASAVHAAEAGEEATSRMQPRLGRASYLGARAVGVPDAGAAAVVVWMKALTPGIG

SEQ ID NO: 60 - DNA, 1701 bp, Burkholderia sp. 383
  CDS (1)..(1701)
atgaaaaagcttgtcaaccgcccgtccgatgtcgtgcgagaaatgctggaaggcatcgcgcggcagtcgccgcatgtcgcgatcctcggcgacgagcacgtgctcgtccgccagccgctgcccgagcccgcgcaacgccccgtcgccatcctgtccggtggcggcagcggccacgagcccgcgcacggcggctatgtcggcgaaggaatgctgagcgcggccgtctgcggcgaagtgttcacgtcgccgtccacagacgccgtgctcgccgcgatccgcgcgagcgccggcccgaacggcgccttgctgatcgtgaagaactacacgggcgaccggctcaatttcgggctcgccgccgaactcgcacgcgccgaaggcattccggtcgagacggtcatcgtcgccgacgacgtatcgctgcgcggccgcgtcgagcgcggccagcggcgcgggatcgccggtaccgtgctgatccacaagctcgccggcgcggcagccgcgcgcgggctgccgctcgcccgcgtcgcggccatcgcgcgcgacgcggcggccgaactcggcacgatgggtgtcgcactcgacggctgcacgatcccgggcgccgacaagtcgggcttcagcctcggcgatcacgagatcgaactcggcctcggcatccatggcgagaaaggcgtcgagcgccgcgcgccgctgccggccgatgcgcttgtcgacacgctgctgtcgagcatcgccgccgatctcgtgctcgaccgcggcgaacgcgttgcgctgttcgtcaacggcctcggcgcgacgccggacatggaactcgcgatcgtgctgcgcgccgcgcacgacaacctgcaccggcgcggcatcgtcgtcgcgcgtgcgtgggccggcacgttcctgtcggcgctgaacatgcccggctgctcgatctcggtgctgcggctgaacgacgaacgcgcggtgctgctcgacgcaccgacgcaggcgcgtgcatggccaggcggcggcgcggtgaatacgcagatccgtgtggcctcggccgccgtgcaggaagcgccgttgccgccgctcgatgcggccggccgcgcgtgggccgcgcgcctgcaaccggcattgcacgcggtcgcgcaaacgctgatcgatcacgagcagacgctgaccgacctcgatgcggcggccggcgacggcgatctcggcgcgagcatgctgcgcgccgcgcaggcgatcctcgcactgccggaaagcgcatacggcacgccggccggcgcgctctcggcgctcggcgccgcgttgcgccgcgcgatcgccggcagctcggggccgttctatgcgaccgcgctgctgcgcgcgtcgcgccggctggccgatatcgccgagccgtccgcacgcgactgggccgcggcgttccgcggcgcggtggattcgatcagcgaactgggcggcgcgcacgccggcgaccggaccatgctcgatgcgctggtcccggccgtcgcggcattcgagcgggcgctcgacaacgatcgcgatcccgccggcgcatggacggccgcggtcgaagccgccgagcacggtgcgcaggaaaccgcacgcatgacgccacgcgccgggcgcgcgagctatctcggcgaacgtgcgatcggcacgccggacggcggcgcggtcgcggtgtcgtattggctgcgtgcgttgcaggcacacatcgggtga

SEQ ID NO: 61 - protein, 566 aa, Burkholderia sp. 383
MKKLVNRPSDVVREMLEGIARQSPHVAILGDEHVLVRQPLPEPAQRPVAILSGGGSGHEPAHGGYVGEGMLSAAVCGEVFTSPSTDAVLAAIRASAGPNGALLIVKNYTGDRLNFGLAAELARAEGIPVETVIVADDVSLRGRVERGQRRGIAGTVLIHKLAGAAAARGLPLARVAAIARDAAAELGTMGVALDGCTIPGADKSGFSLGDHEIELGLGIHGEKGVERRAPLPADALVDTLLSSIAADLVLDRGERVALFVNGLGATPDMELAIVLRAAHDNLHRRGIVVARAWAGTFLSALNMPGCSISVLRLNDERAVLLDAPTQARAWPGGGAVNTQIRVASAAVQEAPLPPLDAAGRAWAARLQPALHAVAQTLIDHEQTLTDLDAAAGDGDLGASMLRAAQAILALPESAYGTPAGALSALGAALRRAIAGSSGPFYATALLRASRRLADIAEPSARDWAAAFRGAVDSISELGGAHAGDRTMLDALVPAVAAFERALDNDRDPAGAWTAAVEAAEHGAQETARMTPRAGRASYLGERAIGTPDGGAVAVSYWLRALQAHIG

SEQ ID NO: 62 - DNA, 1704 bp, Burkholderia thailandensis E264
  CDS (1)..(1704)
atgaagaagctcgtcaaccacccgtccgacgtcgtgcgcgaaatgctggagggcatcgcgcggcagtcgccgcatgtcgcgatgctcggcgacgaacacgtgctgatccggcgccccttgccggagccggcgcggcgtgcggtcgcgatcatctccggcggcggcagcggccacgagccggcgcacggcggctacgtcggcgcggggatgctgagcgcggccgtgtgcggcgaggtcttcacgtcgccgcccgccgatgcggtgctcgccgcgattcgcgcgaccgcgggccagaacggcgcgctcctcatcgtgaagaactacacgggcgatcgcctcaatttcgggctcgcggccgagctcgcgcgcgcgcagggcattccggtcgagatcgtcgtcgtcgcggacgacgtgtcgctgcgcgaactcaccgagcgcgggcgccgccgcggcatcgccggcaccgtgctcgtgcacaagctcgccggcgcggccgccgagcgcggcctcgcgctgcgggaggtggccgccgtcgcgagcgaggcggcggcgaatctcggcacgatgggcgtcgcactcgacggctgcacgattccgggcgccgggcaatcgggcttccgcctcgccgatcacgagatcgagctcggattgggcattcacggcgaaaagggcgtgcagcgcacggcgccgatgccggccgacgcgctgtcggaaacgctcgtggcgacgatcgtcgacgatcaggcgatcgcgcgcggcgatcgggtcgcgcttctggtgaacgggctcggcgcgacgccggacatggagctcggcatcgtgctgcgcgcggcgtacgacagcctgagccggcgtggcgtcgaggtggcgcgcgcgtgggcgggcacgttcctgtccgcgctcgacatgcccggctgctcgatttcgctgctcaagctgaacgatcgcatgctcgaactgctcgacgcgccgacgcaagcgcgagcgtggccgggcggcggcgcggtgaaccgggacattcgcgtggccgccgccgggaccggcgcaggagacggacagccggaatgggcgacggccggcgcggcgggttccgacggactgcggcccgcgctgcatgcggttgccgccgcgctgatcgacagcgagcccgtgttgaccgagctcgattccgtcgccggcgacggcgatctgggcgcgagcatgcgccgcgcggcgaacgcgatgctcgcgctgcccgccgatgcgtatcgagggccggcgaacctgctcgccgcgctgggcatggcgctgcgccgggcgatcgcgggcagctccgggccgttttacgcaacggcgctggtgcgcgcgggccgccggctcgccgatgcatcggcgccgaccgcgcgcgactgggcgagcgcgttccggagcggtgtcgacgcgatcggcgatctgggcggcgcgaagcccggagaccgcacgatgctcgacgccttggtgcccgccgtcgatgcgttcgagcacgcgctgtcggcgggcgggagcgcgagcgatgcgtgggcggcggcggtgcgcgccgcggaggcaggcgcggcgaaaaccgcgggcatgacgccgcgcgcggggcgcgcgagctatctgggcgagcgcgccgtgggctcgcccgacggcggcgcggtggccgtggcgtgctggatgcgcgcgttgcagccgcacgtcgcgtag

SEQ ID NO: 63 - protein, 567 aa, Burkholderia thailandensis E264
MKKLVNHPSDVVREMLEGIARQSPHVAMLGDEHVLIRRPLPEPARRAVAIISGGGSGHEPAHGGYVGAGMLSAAVCGEVFTSPPADAVLAAIRATAGQNGALLIVKNYTGDRLNFGLAAELARAQGIPVEIVVVADDVSLRELTERGRRRGIAGTVLVHKLAGAAAERGLALREVAAVASEAAANLGTMGVALDGCTIPGAGQSGFRLADHEIELGLGIHGEKGVQRTAPMPADALSETLVATIVDDQAIARGDRVALLVNGLGATPDMELGIVLRAAYDSLSRRGVEVARAWAGTFLSALDMPGCSISLLKLNDRMLELLDAPTQARAWPGGGAVNRDIRVAAAGTGAGDGQPEWATAGAAGSDGLRPALHAVAAALIDSEPVLTELDSVAGDGDLGASMRRAANAMLALPADAYRGPANLLAALGMALRRAIAGSSGPFYATALVRAGRRLADASAPTARDWASAFRSGVDAIGDLGGAKPGDRTMLDALVPAVDAFEHALSAGGSASDAWAAAVRAAEAGAAKTAGMTPRAGRASYLGERAVGSPDGGAVAVACWMRALQPHVA

SEQ ID NO: 64 - DNA, 1851 bp, Burkholderia multivorans
  CDS (1)..(1851)
atgtatcgtggcaccgaagcgcgcaccggcgagatccatggcactgctccacgccaccgccgccacggcgcgcgcattcccgcaacccgacagcccgaggcgccggatcggttcggcccgcgggctttcatctgcccggaaaccgtcgccatgaaaaaactcgtgaaccgtccgtccgacgtcgtgcgtgaaatgctcgaaggcattgcgcgacagtcgccgcatctcgcgatgctcggcgacgagcacgtgctcgtccgccgcccgctgcccgaaccgtcgcagcgcacggttgcggtgctgtcgggcggcggcagcgggcacgagcctgcgcacggcggctatgtcggcgacggaatgctcagtgcggccgtgtgcggcgaagtgttcacgtcgccgtccaccgacgcggtgctcgccgcgatccgcgcgacggccggcccgaacggcgcgctgctcgtcgtgaagaactacaccggcgaccggctcaacttcggtctggcagccgaactcgcgcgcgcagaaggcattccggtcgagaccgtgatcgtcgcggacgacgtatcgctgcgcggccgcgtcgagcgcggacagcggcgcggcatcgcgggcaccgtgctgatccacaagctcgcgggcgcggcggccgcgcgcgggctgtcgctgccgcgcgtcgcggcgatcgcgcgcgatgcggccgccgatctcggcacgatgggcgtcgcgctcgacggctgtacgctgccgggcgccgaccagtccggattcagcctcgccgacgacgaaatcgagctcggtctcggcattcatggcgaaaaaggcgtcgaacgcacggcgccgctgccggccgacgcgctcgccgatacgctgctgtccgggatcgtcgccgacctcgtgctcgatcgcggcgaacgcgtcgcgctgctcgtcaacggtctcggcgcgacgcccgacatggagcttgcgatcgtgctgcgcgccgcctacgagaacctgagccgtcgcggcatcgcggtcgagcgcgcgtgggccggtacgttcctgtcggcgctgaacatgcccggctgttcgatctcggtgctgcggctcgacgacgagcgggcggcgctgctcgacgcaccgacgcaagcgcgcgcgtggcccggcggcggcgccgtcaacgcacagatccgcatcgccgccgcggcgccgcaggaaccgtcgccgccgccgctcgacgcggcgggccgcgtgtgggccgagcggctgcgcccggcgctgcacgcggtcgcgcacacgctgatcgaccacgaagcaacgctgaccgagctcgatgccgccgccggcgacggcgacctcggcgcgagcatgcgtcgtgccgcggacgcgatgctcgcgttgccggaaaccgcgtatgcgacgccggccggtgcgctcgccgcgctcggcgccgcgctgcgccgtgcgatcgcgggcagctcgggccccttctacgcgaccgcgctgctgcgcgcatcgcggcgcctggccggcctcgcgcagccgtcggcacgcgactgggccgcggcattgcgcagcgcagccgatgcgatcgccgaactcggcggcgcccgtgccggtgacagaacgatgctcgacgcgctggtgccggccgccgccgcgttcgaccgtgcactcgacgacggtcgcgacagcgccggcgcgtgggcggccgccgtcgacgccgccgagcgcggtgcgcaggacaccgcgcgcatgacgccgcgcgcgggccgtgcgagctatctcggcgaacgcgcgatcggcacgcccgacggcggcgccatcgccgtcgcgtactggctgcgcgcactgctgccgcacgtccgataa

SEQ ID NO: 65 - protein, 616 aa, Burkholderia multivorans
MYRGTEARTGEIHGTAPRHRRHGARIPATRQPEAPDRFGPRAFICPETVAMKKLVNRPSDVVREMLEGIARQSPHLAMLGDEHVLVRRPLPEPSQRTVAVLSGGGSGHEPAHGGYVGDGMLSAAVCGEVFTSPSTDAVLAAIRATAGPNGALLVVKNYTGDRLNFGLAAELARAEGIPVETVIVADDVSLRGRVERGQRRGIAGTVLIHKLAGAAAARGLSLPRVAAIARDAAADLGTMGVALDGCTLPGADQSGFSLADDEIELGLGIHGEKGVERTAPLPADALADTLLSGIVADLVLDRGERVALLVNGLGATPDMELAIVLRAAYENLSRRGIAVERAWAGTFLSALNMPGCSISVLRLDDERAALLDAPTQARAWPGGGAVNAQIRIAAAAPQEPSPPPLDAAGRVWAERLRPALHAVAHTLIDHEATLTELDAAAGDGDLGASMRRAADAMLALPETAYATPAGALAALGAALRRAIAGSSGPFYATALLRASRRLAGLAQPSARDWAAALRSAADAIAELGGARAGDRTMLDALVPAAAAFDRALDDGRDSAGAWAAAVDAAERGAQDTARMTPRAGRASYLGERAIGTPDGGAIAVAYWLRALLPHVR

SEQ ID NO: 66 - DNA, 1920 bp, Escherichia coli
  CDS (1)..(1920)
atgagtggcgcttttaacaacgatggtcggggcatatctcccttaattgcaacctcctgggagcgatgcaataagctgatgaaacgggagacatggaacgtaccacatcaggcccagggcgtgacatttgcttctatttatcggcgtaagaaagcgatgctgacgctcgggcaggctgcgctggaagatgcctgggaatatatggcaccgcgagagtgtgcgctgtttatcctcgatgaaaccgcctgcattctcagccgtaatggcgatccgcaaaccttgcagcagctaagtgcactgggattcaatgacggcacgtattgcgccgagggaattattggtacttgtgcgctatcgttagcggctatctctggtcaggccgtgaaaacgatggccgatcaacatttcaaacaggtactctggaactgggccttttgtgcaacgccgttgtttgacagcaagggccgattgacgggaacaatagcgctggcgtgtccggttgagcaaactaccgcagctgatttgccgttgacgttggcaatcgcccgcgaggtcggaaatttactgctgacggacagtttgctcgctgaaactaaccgtcatttaaatcaacttaatgccctgttagaaagtatggatgatggcgtgattagctgggacgagcagggtaatttgcaatttattaatgcccaggcggcgcgggtcttgcgccttgacgcgacggcaagtcagggacgggcaatcactgaactcttaacgttacccgccgtattgcaacaagcaataaaacaggcacatccgctcaaacacgtagaagcaacctttgaaagccagcaccagtttattgatgcggtgataacccttaaaccgataatagaaacgcagggaaccagctttattttgttgctccatcctgtggaacagatgcggcagttgatgaccagtcaattaggaaaagtcagccataccttcgctcatatgccacaggacgatccgcaaacccgccgcttgattcattttggtcgccaggcggcgcgcagtagctttcctgtcctgctttgtggagaagagggcgtgggcaaggcactgctaagtcaggcaattcataatgaaagcgagcgtgctgcaggtccttatatcgccgtcaattgtgagttatatggtgatgctgcgctggcggaagaatttattggtggcgatcgcacggacaatgaaaatggccgtctgagtcggctggaactggcacacggcggcacgctgtttcttgaaaagattgaatatctggcggtggagttacagtctgctttgcttcaggttatcaagcagggggttatcacgcgactggatgcgcggcgtttaataccaattgatgtcaaagtgattgcaacaacgaccgcggacctcgcaatgctggtggaacaaaatcgttttagtcgccagctgtattacgcgctgcatgcatttgaaattaccatcccgcctctgcgtatgcggcgtggcagcattccggcgctggtgaataacaaattacgcagtcttgaaaaacgcttctctacgcggctgaaaattgatgacgatgccctcgctcgcctggtttcttgtgcatggccaggcaacgattttgaactttacagcgtcatcgagaatcttgctctgagtagtgataacgggcgcattcgcgtcagtgatttgccggaacatctgtttaccgagcaggcgacagatgatgtcagcgccacccgcctttccaccagtctgtcatttgcggaagttgaaaaagaggcaattattaacgcagcccaggtcacaggcggtcgcattcaggaaatgtcggctttacttgggatcggccgcactacgctgtggcggaaaatgaagcaacatggcattgatgcagggcagtttaagcgccgggtatga

SEQ ID NO: 67 - protein, 639 aa, Escherichia coli
MSGAFNNDGRGISPLIATSWERCNKLMKRETWNVPHQAQGVTFASIYRRKKAMLTLGQAALEDAWEYMAPRECALFILDETACILSRNGDPQTLQQLSALGFNDGTYCAEGIIGTCALSLAAISGQAVKTMADQHFKQVLWNWAFCATPLFDSKGRLTGTIALACPVEQTTAADLPLTLAIAREVGNLLLTDSLLAETNRHLNQLNALLESMDDGVISWDEQGNLQFINAQAARVLRLDATASQGRAITELLTLPAVLQQAIKQAHPLKHVEATFESQHQFIDAVITLKPIIETQGTSFILLLHPVEQMRQLMTSQLGKVSHTFAHMPQDDPQTRRLIHFGRQAARSSFPVLLCGEEGVGKALLSQAIHNESERAAGPYIAVNCELYGDAALAEEFIGGDRTDNENGRLSRLELAHGGTLFLEKIEYLAVELQSALLQVIKQGVITRLDARRLIPIDVKVIATTTADLAMLVEQNRFSRQLYYALHAFEITIPPLRMRRGSIPALVNNKLRSLEKRFSTRLKIDDDALARLVSCAWPGNDFELYSVIENLALSSDNGRIRVSDLPEHLFTEQATDDVSATRLSTSLSFAEVEKEAIINAAQVTGGRIQEMSALLGIGRTTLWRKMKQHGIDAGQFKRRV

SEQ ID NO: 68 - DNA, 663 bp, Escherichia coli
  CDS (1)..(663)
atggaactgtatctggatacttcagacgttgttgcggtgaaggcgctgtcacgtatttttccgctggcgggtgtgaccactaacccaagcattatcgccgcgggtaaaaaaccgctggatgttgtgcttccgcaacttcatgaagcgatgggcggtcaggggcgtctgtttgcccaggtaatggctaccactgccgaagggatggttaatgacgcgcttaagctgcgttctattattgcggatatcgtggtgaaagttccggtgaccgccgaggggctggcagctattaagatgttaaaagcggaagggattccgacgctgggaaccgcggtatatggcgcagcacaagggctgctgtcggcgctggcaggtgcggaatatgttgcgccttacgttaatcgtattgatgctcagggcggtagcggcattcagactgtgaccgacttacaccagttattgaaaatgcatgcgccgcaggcgaaagtgctggcagcgagtttcaaaaccccgcgtcaggcgctggactgcttactggcaggatgtgaatcaattactctgccactggatgtggcacaacagatgattagctatccggcggttgatgccgctgtggcgaagtttgagcaggactggcagggagcgtttggcagaacgtcgatttaa

SEQ ID NO: 69 - protein, 220 aa, Escherichia coli
MELYLDTSDVVAVKALSRIFPLAGVTTNPSIIAAGKKPLDVVLPQLHEAMGGQGRLFAQVMATTAEGMVNDALKLRSIIADIVVKVPVTAEGLAAIKMLKAEGIPTLGTAVYGAAQGLLSALAGAEYVAPYVNRIDAQGGSGIQTVTDLHQLLKMHAPQAKVLAASFKTPRQALDCLLAGCESITLPLDVAQQMISYPAVDAAVAKFEQDWQGAFGRTSI

SEQ ID NO: 70 - DNA, 663 bp, Escherichia coli
  CDS (1)..(663)
atggaactgtatctggacaccgctaacgtcgcagaagtcgaacgtctggcacgcatattccccattgccggggtgacaactaacccgagcattatcgctgccagcaaggagtccatatgggaagtgctgccgcgtctgcaaaaagcgattggtgatgagggcattctgtttgctcagaccatgagccgcgacgcgcaggggatggtggaagaagcgaagcgcctgcgcgacgctattccgggtattgtggtgaaaatcccggtgacttccgaaggtctggcagcaattaaaatactgaaaaaagagggtattactacacttggcactgctgtatatagcgccgcacaagggttattagccgcactggcaggggcaaaatacgttgctccgtatgttaaccgcgtagatgcccagggcggagacggcattcgtacggttcaggagctgcaaacgctgttagaaatgcacgcgccagaaagcatggtgctggcagccagctttaaaacgccgcgtcaggcgctggactgtttactggcaggatgtgaatccatcaccctgcccttagatgtagcgcaacaaatgctcaacacccctgcggtagagtcagctatagagaagttcgaacacgactggaatgccgcatttggcactactcatctctaa

SEQ ID NO: 71 - protein, 220 aa, Escherichia coli
MELYLDTANVAEVERLARIFPIAGVTTNPSIIAASKESIWEVLPRLQKAIGDEGILFAQTMSRDAQGMVEEAKRLRDAIPGIVVKIPVTSEGLAAIKILKKEGITTLGTAVYSAAQGLLAALAGAKYVAPYVNRVDAQGGDGIRTVQELQTLLEMHAPESMVLAASFKTPRQALDCLLAGCESITLPLDVAQQMLNTPAVESAIEKFEHDWNAAFGTTHL

SEQ ID NO: 72 - DNA, 1053 bp, Escherichia coli
  CDS (1)..(1053)
atgacagatattgcgcagttgcttggcaaagacgccgacaaccttttacagcaccgttgtatgacaattccttctgaccagctttatctccccggacatgactacgtagaccgcgtaatgattgacaataatcgcccgccagcggtgttacgtaatatgcagacgttgtacaacaccgggcgtctggctggcacaggatatctttctattctgccggttgaccagggcgttgagcactctgccggagcttcatttgctgctaacccgctctactttgacccgaaaaacattgttgaactggcgatcgaagcgggctgtaactgtgtggcgtcaacttacggcgtgctggcgtcggtatcgcggcgttatgcgcatcgcattccattcctcgtcaaacttaatcacaacgagacgctaagttacccgaatacctacgatcaaacgctgtatgccagcgtggagcaggcgttcaacatgggcgcggttgcggttggtgcgactatctattttggctcggaagagtcacgtcgccagattgaagaaatttctgcggcttttgaacgtgcgcacgagctgggtatggtgacagtgctgtgggcctatttgcgtaactccgcctttaagaaagatggcgttgattaccatgtttccgccgacctgaccggtcaggcaaaccatctggcggcaaccatcggtgcagatatcgtcaaacaaaaaatggcggaaaataacggcggctataaagcaattaattacggttacaccgacgatcgtgtttacagcaaattgaccagcgaaaacccgattgatctggtgcgttatcagttagctaactgctatatgggtcgggctgggttgataaactccggcggtgctgcgggcggtgaaactgacctcagcgatgcagtgcgtactgcggttatcaacaaacgcgcaggcggaatggggctgattcttggacgtaaagcgttcaagaaatcgatggctgacggcgtgaaactgattaacgccgtgcaggacgtttatctcgatagcaaaattactatcgcctga

SEQ ID NO: 73 - protein, 350 aa, Escherichia coli [sequence omitted]

SEQ ID NO: 74 - DNA, 1143 bp, Shigella dysenteriae
  CDS (1)..(1143)
atgccgcatttggcactactcatctctaaaggagcaattatggaccgcattattcaatcaccgggtaaatacatccagggcgctgatgtgattaatcgtctgggcgaatacctgaagccgctggcagaactctggttagtggtgggtgacaaatttgttttaggttttgctcaatccactgtcgagaaaagctttaaagatgctggactggtagtagaaattgcgccgtttggcggtgaatgttcgcaaaatgagatcgaccgtctgcgtggcatcgcggagactgcgcagtgtggcgcaattctcggtatcggtggcggaaaaactttcgatactgccaaagcactggcacatttcatgggtgttccggtagcgatcgcaccgacgatcgcctctaccgacgcaccgtgcagcgcattgtctgttatctacaccgatgagggtgagtttgaccgctatctgctgttgccaaataaccctaatatggtcattgtcgacaccaaaatcgtcgctggcgcacctgcacgtctgttagcggcgggtatcggcgatgcgctggcaacctggtttgaagcgcgtgcctgctctcgtagcggcgcgaccaccatggcgggcggcaagtgcacccaggctgcgctggcactggctgaactgtgctacaacaccctgctggaagaaggcgaaaaagcgatgcttgctgccgaacagcatgtagtgactccggcgctggagcgcgtgattgaagcgaacacctatttgagcggtgttggttttgaaagtggtggtctggctgcggcgcacgcagtgcataacggcctgaccgctatcccggacgcgcatcactattatcacggtgaaaaagtggcattcggtacgctgacgcagctggttctggaaaatgcgccggtggaggaaatcgaaaccgtagctgcccttagccatgcggtaggtttgccaataactctcgctcaactggatattaaagaagatgtcccggcgaaaatgcgaattgtggcagaagcggcatgtgcagaaggtgaaaccattcacaacatgcctggcggcgcgacgccagatcaggtttacgccgcactgctggtagctgaccagtatggacaacgtttcctgcaagagtgggaataa

SEQ ID NO: 75 - protein, 380 aa, Shigella dysenteriae
MPHLALLISKGAIMDRIIQSPGKYIQGADVINRLGEYLKPLAELWLVVGDKFVLGFAQSTVEKSFKDAGLVVEIAPFGGECSQNEIDRLRGIAETAQCGAILGIGGGKTFDTAKALAHFMGVPVAIAPTIASTDAPCSALSVIYTDEGEFDRYLLLPNNPNMVIVDTKIVAGAPARLLAAGIGDALATWFEARACSRSGATTMAGGKCTQAALALAELCYNTLLEEGEKAMLAAEQHVVTPALERVIEANTYLSGVGFESGGLAAAHAVHNGLTAIPDAHHYYHGEKVAFGTLTQLVLENAPVEEIETVAALSHAVGLPITLAQLDIKEDVPAKMRIVAEAACAEGETIHNMPGGATPDQVYAALLVADQYGQRFLQEWE

SEQ ID NO: 76 - DNA, 1104 bp, Salmonella typhimurium
  CDS (1)..(1104)
atggatcgcattattcagtcaccaggtaagtatattcagggtgcaaacgtcatcgcgcgtcttggcgattatttaaaaccaatggcgaacaactggctggttgtgggcgataaattcgtgctgggatttgccgaagagacgctgcgcaaaagcctgacggatgccggtttgtcagtagaaatcgccccgtttggcggcgaatgttcgcaaaatgagatcgacaggctgcgcgccgtcgccgaaaaaagtcagtgtggcgccgtactgggtatcggcggcggtaaaacgctggataccgccaaagcgctggcgcactttatgaacgtcccggtcgctatcgcgccgaccatcgcctctaccgacgcaccgtgcagcgcactctcggttatttataccgatgccggtgagtttgaccgttatctgctgctgccgcataacccgaatatggttattgtcgatacgcagatagtggcgggcgcgccggcgcgtctgctggcagccggtatcggcgatgcactggcgacctggtttgaagcgcgcgcctgctcacgcagcggcgccaccacaatggcgggcggcaagtgtacacaggccgcgctggcgctggcggagctatgctataacacgctgatcgaagaaggcgaaaaagccatgttggccgccgaacagcacgtcgtcacgccagcgctggaacgcgtcatcgaagccaacacctacctgagcggggtcggttttgaaagcggcggtctggccgcagcgcacgcgattcataacggtttaacggcgattccggatgcgcaccactattatcacggtgagaaggtcgctttcggtacgctgacgcaactggtgctggaaaacgcgccggtcgaagaaatcgaaaccgttgcggcgctgtgccattccgttggcctgccgattacgctggcgcaactggatattaaacaggatattccggccaagatgcgcaccgtcgcggaagcctcctgcgcagaaggtgaaactattcataacatgcctggcggcgcaacgccggatgaagtgtacgccgcgctgctggtcgccgaccagtacggtcaacgcttcttgcaggaatgggaataa

SEQ ID NO: 77 - protein, 367 aa, Salmonella typhimurium
MDRIIQSPGKYIQGANVIARLGDYLKPMANNWLVVGDKFVLGFAEETLRKSLTDAGLSVEIAPFGGECSQNEIDRLRAVAEKSQCGAVLGIGGGKTLDTAKALAHFMNVPVAIAPTIASTDAPCSALSVIYTDAGEFDRYLLLPHNPNMVIVDTQIVAGAPARLLAAGIGDALATWFEARACSRSGATTMAGGKCTQAALALAELCYNTLIEEGEKAMLAAEQHVVTPALERVIEANTYLSGVGFESGGLAAAHAIHNGLTAIPDAHHYYHGEKVAFGTLTQLVLENAPVEEIETVAALCHSVGLPITLAQLDIKQDIPAKMRTVAEASCAEGETIHNMPGGATPDEVYAALLVADQYGQRFLQEWE

SEQ ID NO: 78 - DNA, 1098 bp, Pseudomonas putida
  CDS (1)..(1098)
atggaccgcgccattcaatcacccggcaaatatgtacaaggggccgatgcgctgcaacggctgggggactacctcaagccgctggcggacagctggctggtgattgccgacaagttcgtgctgggctttgccgaagacaccatccgccaaagcctcagcaaggccgggctggccatggacatcgtcgccttcaacggcgaatgctcgcagggcgaggtcgatcgcctgtgccaactggccacgcaaaacgggcgcagcgccatcgtcggcattggtggcggcaagacgctggacaccgccaaggccgtggcctttttccagaaagtgcccgtggccgtggcccccaccatcgcctccaccgacgcgccctgcagcgcgctgtcggtgctctataccgatgaaggtgagttcgaccgctatctgatgctgcccaccaaccccgccctggtggtggtggacaccgccatcgtcgcccgtgcaccggcgcggctgttggcggccggcattggtgatgccctggccacctggttcgaggcgcgtgccgcatcgcgcagcagcgctgccaccatggccggcggcccggccacgcagaccgcactgaacctggccaggttctgctacgacaccctgctggaagagggtgaaaaagccatgttggccgtgcaggcccaggtggtgacgccggcgctggagcgcatcgtcgaggccaacacctatctgagcggggtcgggtttgaaagcggtggcgtggccgccgcccacgcggtgcacaacggcctgaccgccgtggccgaaacccaccacttctaccacggcgaaaaagtggcgtttggcgtgctggtgcaactggcgctggaaaacgcctccaacgcggaaatgcaggaagtgatgtcgctgtgccacgccgtgggcctgcccatcacgctggcgcagctggacattaccgaagacatccccaccaagatgcgcgccgtggccgagctggcctgcgccccaggcgagaccatccacaacatgcccggcggcgtgacggtggagcaggtctatggcgcgctgctggtggcggaccagctggggcagcattttctggagttttga

SEQ ID NO: 79 - protein, 365 aa, Pseudomonas putida
MDRAIQSPGKYVQGADALQRLGDYLKPLADSWLVIADKFVLGFAEDTIRQSLSKAGLAMDIVAFNGECSQGEVDRLCQLATQNGRSAIVGIGGGKTLDTAKAVAFFQKVPVAVAPTIASTDAPCSALSVLYTDEGEFDRYLMLPTNPALVVVDTAIVARAPARLLAAGIGDALATWFEARAASRSSAATMAGGPATQTALNLARFCYDTLLEEGEKAMLAVQAQVVTPALERIVEANTYLSGVGFESGGVAAAHAVHNGLTAVAETHHFYHGEKVAFGVLVQLALENASNAEMQEVMSLCHAVGLPITLAQLDITEDIPTKMRAVAELACAPGETIHNMPGGVTVEQVYGALLVADQLGQHFLEF

SEQ ID NO: 80 - DNA, 1104 bp, Bacillus coagulans
  CDS (1)..(1104)
atgacgaaaatcattacctctccaagcaagtttatacaaggccccgatgaattgtccaggctttcggcgtatacggaaaggcttggcaaaaaagcatttattattgcggatgattttgtcaccggccttgtcggcaaaacggttgaagaaagctatgccggcaaagaaacggggtatcaaatggcattattcggtggtgagtgttctaaaccggaaatcgaacggctttgtgaaatgagcaaatccgaggaagccgatgtcgttgtcggaatcggcggcggaaaaacattggataccgcaaaagcagtcgggtattacaataacattccggtgattgtcgcgccgaccatcgcttccaccgatgccccgacaagcgccctgtctgttatttacaaagagaacggcgagtttgaagaatacttgatgctgccgctgaacccgacttttgtcattatggatacgaaagtgattgcctctgcccctgcccgcctgctcgtttccggcatgggagatgcgcttgcaacgtattttgaagcgcgcgccactaagcgggcaaataaaacgacgatggcaggcgggcgtgttacggaagcggcgatcgcgcttgcaaaactttgttatgacacgcaaatttcggaaggtttaaaagcaaaactggcagcggaaaaacatcttgttacggaagcagtggaaaaaatcattgaagcgaatacgtatctgagcggaatcggttttgaaagcggcggccttgctgcggcacatgcgatccataatgggcttaccgtgctcgaagaaacccatcatatgtaccacggcgaaaaagtggcattcggtaccctcgcccagctgattttggaagatgcgccgaaagcggaaattgaagaggtggtctccttctgcctgagtgtcggacttcccgtcacgctcggggatttgggcgtgaaagaactgaatgaggaaaagctccgaaaagtggctgaactttcctgtgcggaaggcgaaacgatttataacatgccgtttgaagtcacgcctgaccttgtgtacgcagcaatcgttaccgctgattccgtcgggcggtattataaggaaaaatgggcatga

SEQ ID NO: 81 - protein, 367 aa, Bacillus coagulans
MTKIITSPSKFIQGPDELSRLSAYTERLGKKAFIIADDFVTGLVGKTVEESYAGKETGYQMALFGGECSKPEIERLCEMSKSEEADVVVGIGGGKTLDTAKAVGYYNNIPVIVAPTIASTDAPTSALSVIYKENGEFEEYLMLPLNPTFVIMDTKVIASAPARLLVSGMGDALATYFEARATKRANKTTMAGGRVTEAAIALAKLCYDTQISEGLKAKLAAEKHLVTEAVEKIIEANTYLSGIGFESGGLAAAHAIHNGLTVLEETHHMYHGEKVAFGTLAQLILEDAPKAEIEEVVSFCLSVGLPVTLGDLGVKELNEEKLRKVAELSCAEGETIYNMPFEVTPDLVYAAIVTADSVGRYYKEKWA

SEQ ID NO: 82 - DNA, 999 bp, Escherichia coli
  CDS (1)..(999)
atgaaaacgttaggtgaatttattgtcgaaaagcagcacgagttttctcatgctaccggtgagctcactgctttgctgtcggcaataaaactgggcgccaagattatccatcgcgatatcaacaaagcaggactggttgatatcctgggtgccagcggtgctgagaacgtgcagggcgaggttcagcagaaactcgacttgttcgctaatgaaaaactgaaagccgcactgaaagcacgcgatatcgttgcgggcattgcctctgaagaagaagatgagattgtcgtctttgaaggctgtgaacacgcaaaatacgtggtgctgatggaccccctggatggctcgtccaacatcgatgttaacgtctctgtcggtaccattttctccatctaccgccgcgttacgcctgttggcacgccggtaacggaagaagatttcctccagcctggtaacaaacaggttgcggcaggttacgtggtatacggctcctctaccatgctggtttacaccaccggatgcggtgttcacgcctttacttacgatccttcgctcggcgttttctgcctgtgccaggaacggatgcgcttcccggagaaaggcaaaacctactccatcaacgaaggaaactacattaagtttccgaacggggtgaagaagtacattaaattctgccaggaagaagataaatccaccaaccgcccttatacctcacgttatatcggttcactggtcgcggatttccaccgtaacctgctgaaaggcggtatttatctctacccaagcaccgccagccacccggacggcaaactgcgtttgctgtatgagtgcaacccgatggcattcctggcggaacaagcgggcggtaaagcgagcgatggcaaagagcgtattctggatatcatcccggaaaccctgcaccagcgccgttcattctttgtcggcaacgaccatatggttgaagatgtcgaacgctttatccgtgagttcccggacgcgtaa

SEQ ID NO: 83 - protein, 332 aa, Escherichia coli
MKTLGEFIVEKQHEFSHATGELTALLSAIKLGAKIIHRDINKAGLVDILGASGAENVQGEVQQKLDLFANEKLKAALKARDIVAGIASEEEDEIVVFEGCEHAKYVVLMDPLDGSSNIDVNVSVGTIFSIYRRVTPVGTPVTEEDFLQPGNKQVAAGYVVYGSSTMLVYTTGCGVHAFTYDPSLGVFCLCQERMRFPEKGKTYSINEGNYIKFPNGVKKYIKFCQEEDKSTNRPYTSRYIGSLVADFHRNLLKGGIYLYPSTASHPDGKLRLLYECNPMAFLAEQAGGKASDGKERILDIIPETLHQRRSFFVGNDHMVEDVERFIREFPDA

SEQ ID NO: 84 - DNA, 819 bp, Escherichia coli
  CDS (1)..(819)
atgaccacacgcgtgattgctctcgacttagacggcaccttattgaccccgaaaaagaccctgcttccttcatcgatagaagccctggcccgcgctcgcgaagcaggctatcaattaatcatcgtcacaggtcgccatcacgtcgctattcatcctttttatcaggcgctggcgctggatacacctgctatttgctgtaatggcacctatttgtatgattatcatgcaaaaaccgtgctggaagcggacccaatgcccgttattaaagccctgcaactcattgagatgctgaatgaacaccacattcacggtctgatgtatgtcgatgatgcaatggtctatgagcacccgaccgggcatgtcattcgcacatctaactgggcgcaaaccctgccgccggaacagcgtccgactttcacacaagtcgcttctctggctgaaacggcgcaacaagttaacgccgtatggaagttcgccctgacgcacgatgacctgccgcaattgcagcattttggtaagcatgtcgaacatgaactgggactggagtgtgaatggtcctggcacgatcaggttgatattgcacgcggcggcaacagcaaaggtaaacgtttgacgaaatgggttgaggcgcaaggttggtcgatggaaaacgtcgtggcattcggcgataactttaatgatatcagtatgctggaagccgctggtacaggcgtggcgatgggcaacgccgatgacgcggtaaaagcgcgcgccaacattgtgattggtgataacaccaccgacagcattgcccagttcatttatagccacctgatttaa

SEQ ID NO: 85 - protein, 272 aa, Escherichia coli
MTTRVIALDLDGTLLTPKKTLLPSSIEALARAREAGYQLIIVTGRHHVAIHPFYQALALDTPAICCNGTYLYDYHAKTVLEADPMPVIKALQLIEMLNEHHIHGLMYVDDAMVYEHPTGHVIRTSNWAQTLPPEQRPTFTQVASLAETAQQVNAVWKFALTHDDLPQLQHFGKHVEHELGLECEWSWHDQVDIARGGNSKGKRLTKWVEAQGWSMENVVAFGDNFNDISMLEAAGTGVAMGNADDAVKARANIVIGDNTTDSIAQFIYSHLI

SEQ ID NO: 86 - DNA, 1728 bp, Escherichia coli
  CDS (1)..(1728)
atgatttcaggcattttagcatccccgggtatcgctttcggtaaagctctgcttctgaaagaagacgaaattgtcattgaccggaaaaaaatttctgccgaccaggttgatcaggaagttgaacgttttctgagcggtcgtgccaaggcatcagcccagctggaaacgatcaaaacgaaagctggtgaaacgttcggtgaagaaaaagaagccatctttgaagggcatattatgctgctcgaagatgaggagctggagcaggaaatcatagccctgattaaagataagcacatgacagctgacgcagctgctcatgaagttatcgaaggtcaggcttctgccctggaagagctggatgatgaatacctgaaagaacgtgcggctgacgtacgtgatatcggtaagcgcctgctgcgcaacatcctgggcctgaagattatcgacctgagcgccattcaggatgaagtcattctggttgccgctgacctgacgccgtccgaaaccgcacagctgaacctgaagaaggtgctgggtttcatcaccgacgcgggtggccgtacttcccacacctctatcatggcgcgttctctggaactacctgctatcgtgggtaccggtagcgtcacctctcaggtgaaaaatgacgactatctgattctggatgccgtaaataatcaggtttacgtcaatccaaccaacgaagttattgataaaatgcgcgctgttcaggagcaagtggcttctgaaaaagcagagcttgctaaactgaaagatctgccagctattacgctggacggtcaccaggtagaagtatgcgctaacattggtacggttcgtgacgttgaaggtgcagagcgtaacggcgctgaaggcgttggtctgtatcgtactgagttcctgttcatggaccgcgacgcactgcccactgaagaagaacagtttgctgcttacaaagcagtggctgaagcgtgtggctcgcaagcggttatcgttcgtaccatggacatcggcggcgacaaagagctgccatacatgaacttcccgaaagaagagaacccgttcctcggctggcgcgctatccgtatcgcgatggatcgtagagagatcctgcgcgatcagctccgcgctatcctgcgtgcctcggctttcggtaaattgcgcattatgttcccgatgatcatctctgttgaagaagtgcgtgcactgcgcaaagagatcgaaatctacaaacaggaactgcgcgacgaaggtaaagcgtttgacgagtcaattgaaatcggcgtaatggtggaaacaccggctgccgcaacaattgcacgtcatttagccaaagaagttgatttctttagtatcggcaccaatgatttaacgcagtacactctggcagttgaccgtggtaatgatatgatttcacacctttaccagccaatgtcaccgtccgtgctgaacttgatcaagcaagttattgatgcttctcatgctgaaggcaaatggactggcatgtgtggtgagcttgctggcgatgaacgtgctacacttctgttgctggggatgggtctggacgaattctctatgagcgccatttctatcccgcgcattaagaagattatccgtaacacgaacttcgaagatgcgaaggtgttagcagagcaggctcttgctcaaccgacaacggacgagttaatgacgctggttaacaagttcattgaagaaaaaacaatctgctaa

SEQ ID NO: 87 - protein, 575 aa, Escherichia coli
MISGILASPGIAFGKALLLKEDEIVIDRKKISADQVDQEVERFLSGRAKASAQLETIKTKAGETFGEEKEAIFEGHIMLLEDEELEQEIIALIKDKHMTADAAAHEVIEGQASALEELDDEYLKERAADVRDIGKRLLRNILGLKIIDLSAIQDEVILVAADLTPSETAQLNLKKVLGFITDAGGRTSHTSIMARSLELPAIVGTGSVTSQVKNDDYLILDAVNNQVYVNPTNEVIDKMRAVQEQVASEKAELAKLKDLPAITLDGHQVEVCANIGTVRDVEGAERNGAEGVGLYRTEFLFMDRDALPTEEEQFAAYKAVAEACGSQAVIVRTMDIGGDKELPYMNFPKEENPFLGWRAIRIAMDRREILRDQLRAILRASAFGKLRIMFPMIISVEEVRALRKEIEIYKQELRDEGKAFDESIEIGVMVETPAAATIARHLAKEVDFFSIGTNDLTQYTLAVDRGNDMISHLYQPMSPSVLNLIKQVIDASHAEGKWTGMCGELAGDERATLLLLGMGLDEFSMSAISIPRIKKIIRNTNFEDAKVLAEQALAQPTTDELMTLVNKFIEEKTIC

SEQ ID NO: 88 - DNA, 2903 bp, Artificial
  PtacM fsaB-gldA::Cm
tctagacgctcaagttagtataaaaaagctgaacgagaaacgtaaaatgatataaatatcaatatattaaattagattttgcataaaaaacagactacataatactgtaaaacacaacatatgcagtcactatgaatcaactacttagatggtattagtgacctgtaacagactgcagtggtcgaaaaaaaaagcccgcactgtcaggtgcgggcttttttctgtgttaagcttcgacgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcggatccggccaagctagcttggctctagctagagcgcccggttgacgctgctagtgttacctagcgatttgtatcttactgcatgttacttcatgttgtcaatacctgtttttcgtgcgacttatcaggctgtctacttatccggagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcgaaatcaaataatgattttattttgactgatagtgacctgttcgttgcaacaaattgataagcaatgcttttttataatgccaacttagtataaaaaagcaggcttcaagatcttctctccccatccccctgttcacaattaatcatcggctcgtataatgtgtggaattgtgagcggataacaatttcacacaggacatcatggaactgtatctggacaccgctaacgtcgcagaagtcgaacgtctggcacgcatattccccattgccggggtgacaactaacccgagcattatcgctgccagcaaggagtccatatgggaagtgctgccgcgtctgcaaaaagcgattggtgatgagggcattctgtttgctcagaccatgagccgcgacgcgcaggggatggtggaagaagcgaagcgcctgcgcgacgctattccgggtattgtggtgaaaatcccggtgacttccgaaggtctggcagcaattaaaatactgaaaaaagagggtattactacacttggcactgctgtatatagcgccgcacaagggttattagccgcactggcaggggcaaaatacgttgctccgtatgttaaccgcgtagatgcccagggcggagacggcattcgtacggttcaggagctgcaaacgctgttagaaatgcacgcgccagaaagcatggtgctggcagccagctttaaaacgccgcgtcaggcgctggactgtttactggcaggatgtgaatccatcaccctgcccttagatgtagcgcaacaaatgctcaacacccctgcggtagagtcagctatagagaagttcgaacacgactggaatgccgcatttggcactactcatctctaaaggagcaattatggaccgcattattcaatcaccgggtaaatacatccagggcgctgatgtgattaatcgtctgggcgaatacctgaagccgctggcagaacgctggttagtggtgggtgacaaatttgttttaggttttgctcaatccactgtcgagaaaagctttaaagatgctggactggtagtagaaattgcgccgtttggcggtgaatgttcgcaaaatgagatcgaccgtctgcgtggcatcgcggagactgcgcagtgtggcgcaattctcggtatcggtggcggaaaaaccctcgatactgccaaagcactggcacatttcatgggtgttccggtagcgatcgcaccgactatcgcctctaccgatgcaccgtgcagcgcattgtctgttatctacaccgatgagggtgagtttgaccgctatctgctgttgccaaataacccgaatatggtcattgtcgacaccaaaatcgtcgctggcgcacctgcacgtc

SEQ ID NO: 89 - DNA, 79 bp, Artificial
  atL-Ptac-fsaB
tgcgtgccagacgttcgacttctgcgacgttagcggtgtccagatacagttccatgatgtcctgtgtgaaattgttatc

SEQ ID NO: 90 - DNA, 77 bp, Artificial; identical to SEQ ID NO: 13
  atR-Ptac-fsaB

SEQ ID NO: 91 - DNA, 2847 bp, Artificial
  PtacM2gldA::Cm
tctagacgctcaagttagtataaaaaagctgaacgagaaacgtaaaatgatataaatatcaatatattaaattagattttgcataaaaaacagactacataatactgtaaaacacaacatatgcagtcactatgaatcaactacttagatggtattagtgacctgtaacagactgcagtggtcgaaaaaaaaagcccgcactgtcaggtgcgggcttttttctgtgttaagcttcgacgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcggatccggccaagctagcttggctctagctagagcgcccggttgacgctgctagtgttacctagcgatttgtatcttactgcatgttacttcatgttgtcaatacctgtttttcgtgcgacttatcaggctgtctacttatccggagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcgaaatcaaataatgattttattttgactgatagtgacctgttcgttgcaacaaattgataagcaatgcttttttataatgccaacttagtataaaaaagcaggcttcaagatcttctctccccatccccctgtgtacaattaatcatcggctcgtataatgtgtggaattgtgagcggataacaatttcacacaggagactgccatggaccgcattattcaatcaccgggtaaatacatccagggcgctgatgtgattaatcgtctgggcgaatacctgaagccgctggcagaacgctggttagtggtgggtgacaaatttgttttaggttttgctcaatccactgtcgagaaaagctttaaagatgctggactggtagtagaaattgcgccgtttggcggtgaatgttcgcaaaatgagatcgaccgtctgcgtggcatcgcggagactgcgcagtgtggcgcaattctcggtatcggtggcggaaaaaccctcgatactgccaaagcactggcacatttcatgggtgttccggtagcgatcgcaccgactatcgcctctaccgatgcaccgtgcagcgcattgtctgttatctacaccgatgagggtgagtttgaccgctatctgctgttgccaaataacccgaatatggtcattgtcgacaccaaaatcgtcgctggcgcacctgcacgtctgttagcggcgggtatcggcgatgcgctggcaacctggtttgaagcgcgtgcctgctctcgtagcggcgcgaccaccatggcgggcggcaagtgcacccaggctgcgctggcactggctgaactgtgctacaacaccctgctggaagaaggcgaaaaagcgatgcttgctgccgaacagcatgtagtgactccggcgctggagcgcgtgattgaagcgaacacctatttgagcggtgttggttttgaaagtggtggtctggctgcggcgcacgcagtgcataacggcctgaccgctatcccggacgcgcatcactattatcacggtgaaaaagtggcattcggtacgctgacgcagctggttctggaaaatgcgccggtggaggaaatcgaaaccgtagctgcccttagccatgcggtaggtttgccaataactctcgctcaactggatattaaagaagatgtcccggcgaaaatgcgaattgtggcagaagcggcatgtgcagaaggtgaaaccattcacaacatgcctggcggcgcgacgccagatcaggtttacgccgctctgctggtagccgaccagtacggtcagcgtttcctgcaagagtgggaataa

SEQ ID NO: 92 - DNA, 1740 bp, Artificial
  attR-cat-attL-PtacM2-SD-spacer
tctagacgctcaagttagtataaaaaagctgaacgagaaacgtaaaatgatataaatatcaatatattaaattagattttgcataaaaaacagactacataatactgtaaaacacaacatatgcagtcactatgaatcaactacttagatggtattagtgacctgtaacagactgcagtggtcgaaaaaaaaagcccgcactgtcaggtgcgggcttttttctgtgttaagcttcgacgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcggatccggccaagctagcttggctctagctagagcgcccggttgacgctgctagtgttacctagcgatttgtatcttactgcatgttacttcatgttgtcaatacctgtttttcgtgcgacttatcaggctgtctacttatccggagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcgaaatcaaataatgattttattttgactgatagtgacctgttcgttgcaacaaattgataagcaatgcttttttataatgccaacttagtataaaaaagcaggcttcaagatctctccccatccccctgtgtacaattaatcatcggctcgtataatgtgtggaattgtgagcggataacaatttcacacaggagactgcc

SEQ ID NO: 93 - DNA, 77 bp, Artificial; identical to SEQ ID NO: 12
  atL-ptac-gldA

SEQ ID NO: 94 - DNA, 77 bp, Artificial; identical to SEQ ID NO: 13
  atL-Ptac-fsaB1